SEQUENCE LISTING (26 sequences)

SEQ ID NO: 1 - RNA, 6 nt, Mus musculus
aagugc

SEQ ID NO: 2 - RNA, 23 nt, Mus musculus
caaagugcuuacagugcagguag

SEQ ID NO: 3 - RNA, 23 nt, Mus musculus
uaaagugcuuauagugcagguag

SEQ ID NO: 4 - RNA, 23 nt, Mus musculus
caaagugcuguucgugcagguag

SEQ ID NO: 5 - RNA, 21 nt, Mus musculus
uaaagugcugacagugcagau

SEQ ID NO: 6 - RNA, 23 nt, Mus musculus
caaagugcuaacagugcagguag

SEQ ID NO: 7 - RNA, 23 nt, Mus musculus
caaagugcucauagugcagguag

SEQ ID NO: 8 - RNA, 23 nt, Mus musculus
uaagugcuuccauguuuuaguag

SEQ ID NO: 9 - RNA, 22 nt, Mus musculus
aagugcuuccauguuucagugg

SEQ ID NO: 10 - RNA, 23 nt, Mus musculus
uaagugcuuccauguuuugguga

SEQ ID NO: 11 - RNA, 23 nt, Mus musculus
uaagugcuuccauguuugagugu

SEQ ID NO: 12 - RNA, 22 nt, Mus musculus
uagcagcacguaaauauuggcg

SEQ ID NO: 13 - RNA, 22 nt, Mus musculus
cauugcacuugucucggucuga

SEQ ID NO: 14 - RNA, 23 nt, Mus musculus
cuaugaccuguucgugcagguag

SEQ ID NO: 15 - DNA, 12 bp, Mus musculus
aatagcactttg

SEQ ID NO: 16 - DNA, 12 bp, Mus musculus
aataggtcatag

SEQ ID NO: 17 - DNA, 19 bp, Artificial Sequence
  Primer
gcgtcaacaacatcctgct

SEQ ID NO: 18 - DNA, 20 bp, Artificial Sequence
  Primer
ctcccaggaagatgacaggt

SEQ ID NO: 19 - DNA, 25 bp, Artificial Sequence
  Primer
cgtctctagaaaggtcctacaagaa

SEQ ID NO: 20 - DNA, 20 bp, Artificial Sequence
  Primer
ggctcaggagcaactggtaa

SEQ ID NO: 21 - DNA, 24 bp, Artificial Sequence
  Primer
gggctgtatgagagattgctgatg

SEQ ID NO: 22 - DNA, 22 bp, Artificial Sequence
  Primer
cacggcagtctgagaggatttg

SEQ ID NO: 23 - DNA, 20 bp, Artificial Sequence
  Primer
tccacagcgatatccagaca

SEQ ID NO: 24 - DNA, 20 bp, Artificial Sequence
  Primer
ggacatcaccaggattggac

SEQ ID NO: 25 - DNA, 23 bp, Artificial Sequence
  Primer
atcaagaaggtggtgaagcggaa

SEQ ID NO: 26 - DNA, 24 bp, Artificial Sequence
  Primer
tggaagagtgggagttgctgttga

(19) United States
(12) Patent Application Publication
(10) Pub. No.: US 2011/0189137 A1
(43) Pub. Date: Aug. 4, 2011

(54) METHOD FOR GENERATION AND REGULATION OF IPS CELLS AND COMPOSITIONS THEREOF

(75) Inventor: Tariq M. Rana, San Diego, CA (US)

(73) Assignee: Sanford-Burnham Medical Research Institute, La Jolla, CA (US)

(21) Appl. No.: 12/943,833

(22) Filed: Nov. 10, 2010

Related U.S. Application Data
(60) Provisional application No. 61/260,330, filed on Nov. 11, 2009.

(51) Int. Cl. A61K 35/12; C12N 5/071; C12N 15/85

(52) U.S. Cl. 424/93.7; 435/377; 435/325; 435/455

(57) ABSTRACT

The present invention provides methods for generating induced pluripotent stem (iPS) cells having an increased efficiency of induction as compared with conventional methods. The method includes treating a somatic cell with a nuclear reprogramming factor in combination with an agent that alters microRNA levels or activity in the cell and/or a p21 inhibitor. The invention further provides iPS cells generated by such methods, as well as clinical and research uses for such iPS cells.

### CROSS REFERENCE TO RELATED APPLICATION

This application is based on and claims benefit of priority benefit under 35 U.S.C. §119(e) to U.S. Provisional Application No. 61/260,330 filed on Nov. 11, 2009, the disclosure of which is hereby incorporated by reference in its entirety for all purposes.

### BACKGROUND OF THE INVENTION

1. Field of the Invention

The present invention relates generally to the field of induced pluripotent stem (iPS) cells and more specifically to methods of generating such cells from somatic cells, as well as clinical and research uses for iPS cells generated by such methods.

2. Background Information

Induced pluripotent stem cells (iPSCs) exhibit properties to embryonic stem (ES) cells and were originally generated by ectopic expression of the four nuclear reprogramming factors (4F): Oct4, Sox2, Klf4 and cMyc, in mouse somatic cells. In human cells, besides the original four Yamanaka factors, iPSCs can also be generated with an alternative set of four factors, for example, Oct4 Nanog Lin28 and Sox2. Although many cell types from different tissues have been confirmed to be reprogrammable, a major bottleneck for iPSC derivation and further therapeutic use is the low efficiency of reprogramming, typically from 0.01% to 0.2%. Although tremendous efforts have been focused on screening for small molecules to enhance the reprogramming efficiency as well as developing new methods for iPSC derivation, the mechanisms of how primary fibroblasts are reprogrammed to an ES-like state are still largely unknown.

To understand the mechanism of cellular reprogramming, different approaches have been used. Small molecule based methods have identified that by treating cells with Dnmt1 inhibitors, the reprogramming process can be accelerated. TGF inhibition has also been found to enable faster and more efficient induction of iPSCs which can replace Sox2 and cMyc. Further array analysis has shown that partially reprogrammed iPSCs can be pushed further to become fully reprogrammed when treatment with factors such as methyl transferase inhibitors is provided. Genome-wide analysis of promoter binding and expression induction by the four reprogramming factors demonstrates that these factors have similar targets in iPSCs and mES cells and likely regulate similar sets of genes, and also that targeting of reprogramming factors is altered in partial iPSCs.

More recently, several groups have identified that p53-mediated tumor suppressor pathways may antagonize iPSC induction. Both p53 and its downstream effector p21 are induced during the reprogramming process and decreased expression of both proteins can facilitate iPSC colony formation. Since these proteins are up-regulated in most cells expressing the four reprogramming factors (4F) and cMyc reportedly blocks p21 expression, it remains unclear how ectopic expression of these four factors (4F) overcomes the cellular responses to oncogene/transgenes overexpression and why only a very small population of cells becomes fully reprogrammed.

MicoRNAs are 18-24 nucleotide single stranded small RNAs associated with protein complex called RNA-induced silencing complex (RISC). These small RNAs are usually generated from noncoding regions of gene transcripts and function to suppress gene expression by translational repression. In recent years, microRNAs have been found involved in many different important processes, such as self-renewal gene expression of human ES cells, cell cycle control of embryonic stem (ES) cells, alternative splicing, heart development, among many others. Furthermore, it has been recently reported that ES cell-specific microRNAs can enhance mouse iPSC derivation and replace the function of cMyc during reprogramming. Also hES-specific miR-302 is suggested to alleviate the senescence response due to the four factor expression in human fibroblast. However, since these microRNAs are not expressed until very late stage in the reprogramming process, whether microRNAs play an important role in iPSC induction previously remained unknown.

### SUMMARY OF THE INVENTION

The present invention is based on the seminal discovery that microRNAs are involved during iPSC induction. Interference of the microRNA biogenesis machinery results in significant decrease of reprogramming efficiency. MicroRNA clusters are identified which are highly induced during early stage of reprogramming and functional tests show that introducing such microRNAs into somatic cells enhances induction efficiency. Additionally, key regulators used by reprogramming cells were identified that may be advantageously targeted to significantly increase reprogramming efficiency as well as direct differentiation of iPS cells.

Accordingly, in one embodiment, the present invention provides a method of generating an iPS cell. The method includes contacting a somatic cell with a nuclear reprogramming factor, and contacting the cell with a microRNA that alters RNA levels or activity with the cell, thereby generating an iPS cell. In one aspect, the microRNA or RNA is modified. In another aspect, the microRNA is in a vector. In another aspect, the microRNA is in the miR-17, miR-25, miR-106a, miR let-7 family member (e.g., let-7a, miR 98) or miR-302b cluster. In another aspect, the microRNA is miR-93, miR-106b, miR-21, miR-29a, or a combination thereof.

In one aspect, the microRNA has a polynucleotide sequence comprising SEQ ID NO: 1. In another aspect, the microRNA has a polynucleotide sequence selected from the group consisting of SEQ ID NOs: 2-11. In another aspect, the microRNA regulates expression or activity of p21, Tgfbr2, p53, or a combination thereof. In another aspect, the microRNA regulates Spry 1/2, p85, CDC42, or ERK1/2 pathways.

In one aspect, the nuclear reprogramming factor is encoded by a gene contained in a vector. In another aspect, the nuclear reprogramming factor is a SOX family gene, a KLF family gene, a MYC family gene, SALL4, OCT4, NANOG, LIN28, or a combination thereof. In another aspect, the nuclear reprogramming factor is one or more of OCT4, SOX2, KLF4, C-MYC. In another aspect, the nuclear reprogramming factor comprises c-Myc. In another aspect, induction efficiency is at least doubled as compared without the microRNA.

In one aspect, the somatic cell is contacted with the reprogramming factor prior to, simultaneously with or following contacting with the microRNA. In another aspect, the somatic cell is a mammalian cell. In an additional aspect, the somatic cell is a human cell or a mouse cell.

In another embodiment, the present invention provides a method of generating an iPS cell by contacting a somatic cell with a nuclear reprogramming factor, and an inhibitor of p21 expression or activity.

In another embodiment, the present invention provides a method of generating an induced pluripotent stem (iPS) cell by contacting a somatic cell with an agent that alters RNA levels or activity within the cell, wherein the agent induces pluripotency in the somatic cell, with the proviso that the agent is not a nuclear reprogramming factor, thereby generating an iPS cell. In various embodiments, the RNA is non-coding RNA (ncRNA), including microRNA.

In one aspect of the methods described above, the agent is a polynucleotide, polypeptide, or small molecule. In an additional aspect, the polynucleotide is an antisense oligonucleotide, chemically modified oligonucleotides, locked nucleic acid (LNA), or DNA. In another aspect, the polynucleotide is RNA. In an additional aspect, the RNA is selected from the group consisting of microRNA, dsRNA, siRNA, stRNA, or shRNA. In another aspect, the somatic cell is a mouse embryonic fibroblast (MEF).

In various aspects, the agent that alters RNA can inhibit p21, Tgfbr2, p53, or a combination thereof, for expression or activity. In one aspect, the agent may be a polynucleotide, polypeptide, or small molecule. In another aspect the agent or the inhibitor of p21, Tgfbr2, and/or p53 is an RNA molecule, including microRNA, dsRNA, siRNA, stRNA, or shRNA, or antisense oligonucleotide. In an exemplary aspect the agent or the inhibitor of p21, Tgfbr2, and/or p53 is a microRNA molecule and encoded by a polynucleotide contained in a recombinant vector introduced into the cell.

In various aspects, the microRNA may be a microRNA included in a cluster that exhibits an increase or decrease in activity or expression during induction of an iPSC or differentiation thereof. In one aspect, induction efficiency is at least doubled as compared without the agent. In another aspect, induction efficiency is at least three folds as compared without the agent. In another aspect, induction efficiency is at least five folds as compared without the agent. In one aspect the microRNA may be one or more microRNAs in the miR-17, miR-25, miR-106a, or miR-302b cluster, including miR-93, miR-106b, miR-21, miR-29a, miR-let-7 family member (e.g., let-7a; miR 98) or a combination thereof. In a related aspect, the microRNA has a polynucleotide sequence comprising SEQ ID NO: 1, which has been determined to be conserved between various microRNAs, e.g., those of SEQ ID NOs: 2-11, corresponding to microRNA species within miR-17, miR-25, miR-106a, and miR-302b clusters. In one aspect, the microRNA has a polynucleotide sequence selected from the group consisting of SEQ ID NOs: 2-11.

In various aspects, the nuclear reprogramming factor is encoded by a gene contained in a recombinant vector introduced into the cell. In another aspect, the agent inhibits expression or activity of p21, Tgfbr2, p53, or a combination thereof. In another aspect, the agent regulates Spry 1/2, p85, CDC42, or ERK1/2 pathways.

In various aspects, the nuclear reprogramming factor is encoded by one or more of a SOX family gene, a KLF family gene, a MYC family gene, SALL4, OCT4, NANOG, LIN28, or a combination thereof. In an exemplary aspect, the nuclear reprogramming factor is one or more of OCT4, SOX2, KLF4, C-MYC. In another aspect, the at least one nuclear reprogramming factor comprises c-Myc. In an additional aspect, c-Myc enhances reprogramming at least partly by repressing at least one miRNA.

In another embodiment, the invention provides an iPS cell or population of such cells produced using the method described herein. In another embodiment, the invention provides an enriched population of induced pluripotent stem (iPS) cells produced by the method described herein.

Similarly, in another embodiment, the invention provides a differentiated cell derived by inducing differentiation of an iPSC generated using the method described herein. In one aspect, the somatic cell is derived by inducing differentiation by contacting the iPSC with an RNA molecule or antisense oligonucleotide. In one aspect, the RNA molecule is selected from the group consisting of microRNA, dsRNA, siRNA, stRNA, or shRNA.

In another embodiment, the invention provides a method of treating a subject with iPS cells generated using the method described herein. The method includes inducing a somatic cell of the subject into an induced pluripotent stem (iPS) cell using the method described herein, inducing differentiation of the iPS cell, and introducing the differentiated cell into the subject, thereby treating the condition.

In another embodiment, the present invention provides a method for evaluating a physiological function of an agent using an iPS cell generated by the method described herein or a somatic cell derived therefrom. In one aspect, the method includes treating an induced pluripotent stem (iPS) cell produced using the methods described herein and evaluating a change in at least one cellular function resulting from the agent. In another aspect, the method includes treating a differentiated cell derived by inducing differentiation of the pluripotent stem cell described herein with the agent and evaluating a change in cellular function resulting from the agent.

In another embodiment, the present invention provides a method evaluating toxicity of a compound using an iPS cell generated by the method described herein or a somatic cell derived therefrom. In one aspect, the method includes treating an induced pluripotent stem (iPS) cell produced using the method described herein with the compound and evaluating the toxicity of the compound. In another aspect, the method includes treating a differentiated cell derived by inducing differentiation of the pluripotent stem cell described herein with the compound and evaluating the toxicity of the compound.

In another embodiment, the present invention provides a method of generating an induced pluripotent stem (iPS) cell. The method includes contacting a somatic cell with at least one nuclear reprogramming factor; and contacting the cell with an inhibitor of p21, Tgfbr2, p53, or a combination thereof, for expression or activity. In one aspect, the inhibitor inhibits expression and/or activity of p21. In another aspect, the inhibitor inhibits expression and/or activity of Tgfbr2. In another aspect, the inhibitor inhibits expression and/or activity of p53.

In another embodiment, the present invention provides a method of generating an induced pluripotent stem (iPS) cell. The method includes contacting a somatic cell with an agent that alters RNA levels or activity within the cell, wherein the agent induces pluripotency in the somatic cell, with the proviso that the agent is not a nuclear reprogramming factor, thereby generating an iPS cell.

In another embodiment, the present invention provides a method of treating a subject. The method includes generating an induced pluripotent stem (iPS) cell from a somatic cell of the subject by the method described herein; inducing differentiation of the iPS cell; and introducing the cell into the subject, thereby treating the condition.

In another embodiment, the present invention provides a use of microRNA for increasing efficiency of generating of iPS cells. In one aspect, the microRNA is selected from the group consisting of miR-17, miR-25, miR-93, miR-106a, miR-106b, miR-21, miR-29a, miR-302b cluster, or a combination thereof. In another aspect, the microRNA is in the miR-17, miR-25, miR-106a, or miR-302b cluster. In another aspect, the microRNA is miR-93, miR-106b, miR-21, miR-29a, or a combination thereof.

In another embodiment, the present invention provides a combination of miR sequences selected the group consisting of miR-17, miR-25, miR-93, miR-106a, miR-106b, miR-21, miR-29a, miR-302b cluster, miR let-7 family member or a combination thereof. In another aspect, the microRNA is in the miR-17, miR-25, miR-106a, or miR-302b cluster. In another aspect, the microRNA is miR-93, miR-106b, miR-21, miR-29a, or a combination thereof.

### BRIEF DESCRIPTION OF THE DRAWINGS

FIG. 1 shows the involvement of RNAi machinery in mouse iPSC induction. FIGS. 1a, 1b, and 1c illustrate knock-down of mouse RNAi machinery genes Ago2, Drosha, and Dicer and by shRNAs, respectively. Both mRNA and protein level of targeted genes are analyzed by RT-qPCR as shown in the histograms and corresponding western blots. Primary mouse embryonic fibroblasts (MEFs) are transduced with four factors plus shRNA targeting Drosha, Dicer and Ago2. MEFs are transduced with lentiviral shRNAs plus 4 μg/μl polybrene, and total RNAs or proteins are harvested at day 3 post-transduction. mRNA and protein levels of targeted genes are analyzed by RT-qPCR and Western blotting, respectively. pLKO is the empty vector control for the shRNA lentiviral vectors. pGIPZ is a lentiviral vector expressing a non-targeting shRNA. FIG. 1d shows knock-down of Ago2 decreases iPSC induction by OSK. Colonies are stained and quantified for AP at day 21 post transduction. Error bar represent standard deviation from duplicate wells. FIG. 1e shows GFP+ colony quantification of iPSC with shAgo2. GFP+ colonies are quantified at day 21 post transduction. Error bar represent standard deviation from duplicate wells. FIG. 1f shows that knock-down of Ago2 dramatically decreases iPSC induction by 4F. Primary MEFs are transduced with the four reprogramming factors (OSKM (4F)) plus shRNA Ago2. Colonies can be stained at day 14 post transduction for alkaline phosphatase, which is a marker for mES/iPS cells. pLKO and pGIPZ vectors served as negative controls.

FIG. 2 shows the induction of microRNA clusters miR-17, 25, 106a and 302b during early stage of reprogramming. FIG. 2a shows a graphical representation illustrating expression induction of 10 microRNA clusters in the early stage after four factor transduction. miR RT-qPCR is used to quantify the expression changes of representative microRNAs of 10 clusters which are highly expressed in ES cells. Total RNAs from starting MEFs and MEFs with 4F at day 4 post infection are analyzed. Dark bars of the histogram show day 4 MEFs after infection, while blank bars show starting MEFs. Asterisks indicate induced microRNAs. FIG. 2b shows a seed region comparison of different miR clusters induced at day 4 post 4F transduction. Similar seed regions are underlined. FIG. 2c shows a graphical representation of induction of microRNAs. Representative microRNAs can be induced with different combination of four factors. MicroRNA expression is quantified after 4 days post transduction. 4F, OSK, OS and single factors are used to analyze which factors were responsible for miR expression change.

FIG. 3 shows the enhanced induction of iPSC by miR-93 and miR-106b. FIG. 3a is a pictorial representation showing a reprogramming assay timeline. MicroRNA mimics are transfected on day 0 and day 5 at a final concentration of 50 nM. GFP+ colonies are quantified at day 11 for 4F induction and day 15-20 for OSK three factor iPSC induction. FIG. 3b is a graphical representation showing miR-93 and miR-106b mimic enhance iPSC induction with 4F induction. Oct4-GFP MEFs are transfected with 50 nM indicated microRNAs. GFP+ colonies are quantified at day 11 post transduction. Fold-induction and error bars were calculated from three independent experiments using triplicate wells. FIG. 3c is a graphical representation showing identification of the enhancing effect of miR-93 and miR-106b using OSK system. MicroRNA mimics are transfected as in the 4F experiments. GFP+ colonies are quantified on days 15-20. Error bars represent standard deviation from three independent experiments with triplicate wells. FIG. 3d is a graphical representation showing the effect of inhibition of microRNAs on reprogramming efficiency. Inhibitors of miR-93 and miR-106b dramatically decrease reprogramming efficiency. MicroRNA inhibitors are also transfected at a final concentration of 50 nM and maintain the same experiment timeline as miR mimic transfection. Error bars represent standard deviation from three independent experiments with triplicate wells.

FIG. 4 shows the characterization of iPSC clones derived from miR mimic experiments, where expressions via RT-PCR of different endogenous ES markers are analyzed. Total RNAs are isolated from iPS cell lines at day 3 post-passage. ES cell-specific markers such as Eras, ECat I, Nanog, and endogenous Oct4 expression are analyzed by RT-PCR.

FIG. 5 shows the targeting of mouse p21 and Tgfbr2 by miR-93 and miR-106b. FIG. 5a shows that miR-93 and 106b transfection decreases p21 protein levels. Oct4-GFP MEFs are transfected with 50 nM miR mimics and harvested 48 hours after transfection for Western analysis. Actin is used as the loading control. FIG. 5b shows that p21 is knocked down efficiently by siRNA. P21 siRNA- and control-transfected MEFs are harvested at 48 hr and RT-qPCR, and western blotting is undertaken to verify p21 expression. p21 mRNAs are normalized to GAPDH. FIG. 5c shows that knock-down of p21 by siRNA enhances iPSC induction. MEFs are infected with 4F virus, and siRNAs are transfected following the same timeline as microRNAs mimic transfection. GFP+ colonies are quantified at day 11. Error bars represent at least two independent experiments using triplicate wells. FIG. 5d shows that miR-93 and 106b transfection decreases Tgfbr2 expression. Transfected cells are harvested at 48 hr for western blotting. FIG. 5e shows that Tgfbr2 is knocked down by siRNAs. Relative Tgfbr2 mRNA levels are normalized to those of Gapdh. FIG. 5f shows that knock-down of Tgfbr2 by siRNAs enhances iPSC induction. Error bars represent at least three independent experiments using triplicate wells.

FIG. 6 shows the enhancement of reprogramming by microRNAs. FIG. 6a shows that miR-17 and miR-106a can enhance reprogramming efficiency, but not miR-16. MiR-17 and miR-106a mimics are transfected into MEFs at a final concentration of 50 nM. GFP+ colonies are quantified at day 11 post-transduction. Error bars represent two independent experiments with triplicate wells. FIG. 6b shows that miR-17 and 106a target p21. p21 Western blotting is performed 2 days after transfection of microRNA mimics into MEFs. miR-17 and miR-106a target Tgfbr2 expression. microRNA mimics are transfected into MEFs at 50 nM final concentration. FIG. 6c shows that miR-17 and 106a target Tgfbr2. Western blotting is performed 2 days post transfection. FIG. 6d shows a model for the role for microRNAs during iPSC induction. Several microRNAs, including miR-17, 25 and 106a clusters, are induced during early stages of reprogramming. These microRNAs facilitate full reprogramming by targeting factors that antagonize the process, such as p21 and other unidentified proteins. Up and down represent the potential different stages and barriers during reprogramming process and dashed line indicates that barriers for reprogramming which are lowered upon microRNAs induction in reprogrammed cells.

FIG. 7 is a graphic diagram depicting the dose response of miR-93 and miR-106b on mouse iPSC induction. Oct4-GFP MEFs are transfected with different concentrations (5, 15 and 50 nM) of microRNAs. Mimic control siRNA are used as a control. GFP+ colonies are quantified at day 11 post transduction. Data represents triplicate wells in 12-well plates.

FIG. 8 shows p21 expression induced during iPSC induction. FIG. 8a shows western blot analysis using different systems (from left to right: OSKM, OSK, OS, Klf4, cMyc, and MEF Control) of p21 expression. P21 expression is induced by Klf4 and cMyc. MEFs infected with 4F, OSK, OS, Klf4 and cMyc are harvested at day 5 post transduction for western blotting analysis. FIG. 8b shows a graphical diagram showing expression confirmation of different transgenes in infected MEFs.

FIG. 9 shows inhibition of reprogramming using OSK three factors by p21 overexpression. FIG. 9a is a graphical diagram of AP+ colony quantification of iPSC from OSK induction and p21 overexpression. Induced cells are stained for alkaline phosphatase at day 21. p21 virus is introduced at the same time with OSK. FIG. 9b is a graphical diagram of GFP+ colony quantification of iPSC from OSK induction and p21 overexpression.

FIG. 10 shows direct regulation by miRNAs of p21 expression. FIG. 10a is a pictorial representation showing two potential sites found in the p21 mRNA 3′UTR. Mutations are introduced to the first site (conserved site) to disrupt the binding affinity of miR-93 and 106b. FIG. 10b is a graphical diagram showing quantification of pGL3-p21 luciferase reporter expression in Hela cells. Hela cells are transfected with pGL3-p21 and pRL-TK as well as microRNAs for 48 hrs before harvesting. Results are normalized to pRL-TK level in transfected cells.

FIG. 11 shows direct regulation by miRNA of Tgfbr2 expression. FIG. 11a is a pictorial representation showing two potential sites found in the Tgfbr2 mRNA 3′UTR. FIG. 11b is a graphical diagram showing quantification of luciferase reporter expression in Hela cells, as carried out similarly as the p21 experiment. Results are normalized to pRL-TK level in transfected cells.

FIG. 12 shows relative Tgfbr2 mRNA levels in the presence of various miRNAs as indicated.

FIG. 13 shows that shRNA are actively expressed in shAgo2 infected MEFs. FIG. 13a shows the shAgo2 levels and FIG. 13b shows the shRNA levels. FIG. 13c shows expressions of ES-specific markers in Ago2 infected MEFs.

FIG. 14 shows relative miRNA expressions at days 0, 4, 8, and 12 following transduction of the OSKM factors.

FIG. 15 shows the effects of miR-93 mimic upon relative levels of miR-93.

FIG. 16a shows that miR inhibitors can decrease target miR expressions. FIG. 16b further shows miR inhibitor's effects during different stages of the reprogramming process.

FIG. 17 shows levels of promoter methylation of endogenous Nanog loci when miR-93 or miR-106b is introduced.

FIGS. 18a and 18b show that genes significantly decreased upon miR-93 transfection can show a threefold enrichment of genes which are lowly expressed in iPSCs, while genes which are increased upon miR-93 transfection do not show such enrichment.

FIG. 19a shows relative Tgfbr2 mRNA levels upon introduction of miR-93 using either mRNA array or RT-qPCR analysis. FIG. 19b shows relative mRNA levels upon introduction of miR-25, miR-93, or miR-106b.

FIG. 20 shows inhibition of MEF-enriched microRNAs, miR-21 and miR-29a, enhances iPS cell reprogramming efficiency. FIG. 20a shows that miR-29a, miR-21, and let7a are highly expressed in MEFs. Total RNAs are isolated from Oct4-EGFP MEFs and mouse ES cells and resolved by gel electrophoresis. Specific radioactive-labeled probes against the indicated miRNAs are used to detect signals. U6 snRNA serves as a loading control. FIG. 20b shows that miRNA inhibition enhances reprogramming efficiency. Oct4-EGFP MEFs are transduced with OSKM. GFP-positive colonies are identified and counted by fluorescence microscopy at day 14 post-transduction, GFP+ colony number is normalized to the number of anti miR non-targeting control treatment and is reported as fold-change. Error bars represent the standard deviation of three independent experiments. *p value<0.05.

FIG. 21 shows that c-Myc is the primary repressor of MEF-enriched miRNAs during reprogramming. FIG. 21a shows Northern analysis of selected miRNAs at day 5 post reprogramming. Oct4-EGFP MEFs are transduced with a single factor or various combinations of reprogramming factors, as indicated. 1F, one factor; 2F, two factors; 3F, three factors; OSKM: Oct4, Sox2, Klf4, and c-Myc. U6 is used as a loading control RNA. Total RNA from embryonic stem cells (ES) serve as negative control to MEF and transduced cells. Various probes are used to detect specific miRNAs as indicated on the right side. MiR-291 blotting is a positive control for ES RNA.

FIG. 21b shows quantitative representation of miRNA expression in the presence of various reprogramming factors. Signal intensity is normalized to intensity of U6 snRNA. The expression ratio is calculated as the percent expression of each miRNA relative to expression in MEFs, which is arbitrarily set to 100%. Various miRNAs are quantified (from panel A) and indicated on the right side.

FIG. 21c shows real time RT-PCR analysis of selected miRNAs in Oct4-EGFP MEFs at various time points following OSK- or OSKM-reprogramming. RNA is isolated at the indicated day (D) after transduction for real time RT-PCR analysis. Signals are normalized to U6 and are shown as a percentage of miRNAs expressed in MEFs, which is arbitrarily set to 100. Error bars represent standard deviations of two independent experiments.

FIG. 22 shows inhibition of miR-21 or miR-29a enhances iPS cell reprogramming by decreasing p53 protein levels and upregulating p85α and CDC42 pathways. FIG. 22a shows Western analysis of expression of p53, CDC42, and p85α following inhibition of various miRNAs. 1×10⁵ Oct4-EGFP MEFs are transfected with indicated miRNA inhibitors. Cells are harvested and analyzed 5 days later. FIG. 22b shows quantitative representation of protein expression in the presence of indicated miR inhibitors. Signal intensity is normalized to GAPDH intensity, and shown as a percentage relative to expression in control (NT) cells, which was set arbitrarily to 100. Error bars show standard deviation of at least three independent experiments. * p value<0.05.

FIG. 22c shows immunoblot analysis of p53, CDC42, and p85α expression following inhibition of various miRNAs and OSKM transduction. 1×10⁵ Oct4-EGFP MEFs are transfected with indicated miRNA inhibitors. Cells are harvested 5 days later and analyzed by immunoblot. Signal intensity is normalized as described in (B). Error bars show standard deviation of at least three independent experiments. * p value<0.05. FIG. 22d shows that depleting miR-29a or p53 enhances reprogramming efficiency. 4×10⁴ Oct4-EGFP MEFs are transfected with indicated siRNAs and miRNA inhibitors, as well as OSKM reprogramming factors. GFP-positive cells are counted at day 12 post-transduction. Error bars show standard deviation of at least three independent experiments. * p value<0.05.

FIG. 23 shows that depleting miR-21 and miR-29a promotes reprogramming efficiency by downregulating the ERK1/2 pathway. FIG. 23a shows Western analysis of phosphorylated and total ERK1/2 following inhibition of various miRNAs in MEFs. 1×10⁵ Oct4-EGFP MEFs are transfected with indicated miRNA inhibitors, harvested 5 days later, and immunoblotted. Signal intensity normalized to Actin, and shown as percentage relative to expression of anti miR NT control. Error bars show standard deviation of three independent experiments. * p value<0.05; ** p value<0.005. FIG. 23b shows that depleting miR-21 and miR-29a increases Spry1 protein levels. Western blot analysis of Spry1 expression ratio is shown. MEFs are transfected with various miRNA inhibitors as indicated. Cells are harvested at day 5 post transfection for Western blot analysis. Signal intensity normalized to Actin and shown as describe in FIG. 23a. Error bars represent standard deviations of three independent experiments. * p value<0.05; ** p value<0.005.

FIG. 23c shows fold-change in reprogramming efficiency following ERK1/2 or GSK3β knock-down. 4×10⁴ Oct4-EGFP MEFs are transfected with indicated siRNAs, as well as OSKM. GFP-positive cells are counted two weeks later. Transfection with siNT serves as control for the reprogramming efficiency. Error bars indicate standard deviation of three independent experiments. ** p value<0.005. FIG. 23d shows Western analysis of phosphorylated and total GSK-3 β following inhibition of various miRNAs in MEFs. 1×10⁵ Oct4-EGFP MEFs are transfected with indicated miRNA inhibitors, harvested 5 days later, and analyzed by immunoblot. Signal intensity normalized as described in FIG. 23a. Error bars show standard deviation of three independent experiments.

FIG. 24 shows a schematic representation illustrating that c-Myc enhances reprogramming by down-regulating the MEF-enriched miRNAs, miR-21 and miR-29a. The p53 and ERK1/2 pathways function as barriers to reprogramming, and miR-21 and miR-29a indirectly activate those pathways through down-regulating CDC42, p85α, and Spry1. The cross talk between miR-21/p53 and miR-29a/ERK1/2 pathways is also shown. c-Myc represses expression of these miRNAs and in turn compromises induction of ERK1/2 and p53. The dotted lines indicate p53 and ERK1/2 effects on iPS reprogramming.

FIG. 25 shows inhibition of miR-21 enhances iPS cell reprogramming by OSK. Inhibitors of miRNAs are introduced into Oct4-MEFs during reprogramming with OSK. GFP-positive colonies are counted at various time points post-transduction. Error bars represent standard deviation of two independent experiments.

FIG. 26 shows that inhibition of miRNA does not alter apoptosis or proliferation rates during reprogramming. FIG. 26a shows that inhibitors of miRNA are introduced into Oct4-MEFs during reprogramming with OSKM. Cells are collected at 8-9 days post transduction. Apoptosis is evaluated using a PE Annexin V Apoptosis Detection Kit I (BD Pharmingen; Cat# 559763) and 7-Amino-Actinomycin (7-AAD). The signal is detected by FACS. Error bars represent standard deviation of three independent experiments. FIG. 26b shows that miRNA inhibitors are introduced into Oct4-MEFs during reprogramming with OSKM. Cells are collected at 8˜9 days post transduction. One day before collection, cells are treated with 5-ethynyl-2′-deoxyuridine (Edu) using Click-iT Edu Imaging Kits (Invitrogen; Cat# C10337). The signal is detected by FACS. Error bars represent standard deviation of three independent experiments.

### DETAILED DESCRIPTION OF THE INVENTION

The present invention is based on the discovery of key regulatory mechanisms involved in iPSC induction. A key aspect being the discovery of a link between cellular microRNAs to the induction of iPSCs. This is evidenced by the observation that interference of the microRNA biogenesis machinery by knock-down of key microRNA pathway proteins can result in significant decrease of reprogramming efficiency. In particular, at least three microRNA clusters are revealed, miR-17˜92, 106b˜25 and 106a˜363, that are highly induced during early stages of reprogramming. Several microRNAs, such as miR-93 and miR-106b which have very similar seed regions greatly enhance iPSC induction by targeting p21 expression allowing derived clones to reach a fully reprogrammed state.

The present invention provides that microRNAs can function directly in iPSC induction and that interference with the microRNA biogenesis machinery significantly decreases reprogramming efficiency. The present invention provides three clusters of microRNAs, miR-17˜92, miR-106b˜0.25 and miR-106a˜363, which are highly induced during early stages of reprogramming. Functional analysis demonstrated that introducing these microRNAs into MEFs enhanced Oct4-GFP+ iPSC colony formation. The present invention also provides that Tgfbr2 and p21, both of which inhibit reprogramming, are directly targeted by these microRNAs and that blocking their activity significantly decreased reprogramming efficiency. The present invention provides that miR-93 and miR-106b are key regulators of reprogramming activity.

Before the present compositions and methods are described, it is to be understood that this invention is not limited to particular compositions, methods, and experimental conditions described, as such compositions, methods, and conditions may vary. It is also to be understood that the terminology used herein is for purposes of describing particular embodiments only, and is not intended to be limiting, since the scope of the present invention will be limited only in the appended claims.

As used in this specification and the appended claims, the singular forms “a”, “an”, and “the” include plural references unless the context clearly dictates otherwise. Thus, for example, references to “the method” includes one or more methods, and/or steps of the type described herein which will become apparent to those persons skilled in the art upon reading this disclosure and so forth.

Unless defined otherwise, all technical and scientific terms used herein have the same meaning as commonly understood by one of ordinary skill in the art to which this invention belongs. Although any methods and materials similar or equivalent to those described herein can be used in the practice or testing of the invention, the preferred methods and materials are now described.

As discussed herein, the discovery that microRNAs are involved in reprogramming process and iPSC induction efficiency leads to the ability of one to greatly enhance iPSC induction efficiency by manipulating the level of these microRNAs in the cells. Accordingly, the present invention provides a method of generating an iPS cell having improved induction efficiency as compared to know methods. The method includes contacting a somatic cell with a nuclear reprogramming factor, and an agent that alters microRNA levels or activity within the cell, with the proviso that the agent is not a nuclear reprogramming factor, thereby generating an iPS cell.

The present invention is also based on the discovery of regulatory proteins that are directly involved in reprogramming process and iPSC induction efficiency. One such protein is p21, a small protein with only 165 amino acids, which has long been known as a tumor suppressor during cancer development by causing p53-dependent G1 growth arrest and promoting differentiation and cellular senescence. Inhibition of p21 expression by microRNAs during iPSC induction has been shown herein to increase induction efficiency. Accordingly, in one embodiment, the present invention provides a method of generating an iPS cell by contacting a somatic cell with a nuclear reprogramming factor, and an inhibitor of p21 expression or activity.

Given the regulatory involvement of RNA in generation of iPSC, it is contemplated that induction may occur using agents that regulate RNA levels other than nuclear reprogramming factors. Accordingly, the present invention provides a method of generating an induced pluripotent stem (iPS) cell by contacting a somatic cell with an agent that alters RNA levels or activity within the cell, wherein the agent induces pluripotency in the somatic cell, with the proviso that the agent is not a nuclear reprogramming factor, thereby generating an iPS cell. In various embodiments, the RNA is non-coding RNA (ncRNA), such microRNA.

In various embodiments, one or more nuclear reprogramming factors can be used to induce reprogramming of a differentiated cell without using eggs, embryos, or ES cells. Efficiency of the induction process is enhanced by utilizing an agent that alters microRNA levels or activity within the cell during the induction process. The method may be used to conveniently and highly reproducibly establish an induced pluripotent stem cell having pluripotency and growth ability similar to those of ES cells. For example, the nuclear reprogramming factor may be introduced into a cell by transducing the cell with a recombinant vector comprising a gene encoding the nuclear reprogramming factor along with a recombinant vector comprising a polynucleotide encoding an RNA molecule, such as a microRNA. Accordingly, the cell can express the nuclear reprogramming factor expressed as a product of a gene contained in the recombinant vector, as well as expressing the microRNA expressed as a product of a polynucleotide contained in the recombinant vector thereby inducing reprogramming of a differentiated cell at an increased efficiency rate as compare to use of the nuclear reprogramming factor alone.

As used herein, pluripotent cells include cells that have the potential to divide in vitro for an extended period of time (greater than one year) and have the unique ability to differentiate into cells derived from all three embryonic germ layers, including the endoderm, mesoderm and ectoderm.

Somatic cells for use with the present invention may be primary cells or immortalized cells. Such cells may be primary cells (non-immortalized cells), such as those freshly isolated from an animal, or may be derived from a cell line (immortalized cells). In an exemplary aspect, the somatic cells are mammalian cells, such as, for example, human cells or mouse cells. They may be obtained by well-known methods, from different organs, such as, but not limited to skin, lung, pancreas, liver, stomach, intestine, heart, reproductive organs, bladder, kidney, urethra and other urinary organs, or generally from any organ or tissue containing living somatic cells. Mammalian somatic cells useful in the present invention include, by way of example, adult stem cells, sertoli cells, endothelial cells, granulosa epithelial cells, neurons, pancreatic islet cells, epidermal cells, epithelial cells, hepatocytes, hair follicle cells, keratinocytes, hematopoietic cells, melanocytes, chondrocytes, lymphocytes (B and T lymphocytes), erythrocytes, macrophages, monocytes, mononuclear cells, fibroblasts, cardiac muscle cells, other known muscle cells, and generally any live somatic cells. In particular embodiments, fibroblasts are used. The term somatic cell, as used herein, is also intended to include adult stem cells. An adult stem cell is a cell that is capable of giving rise to all cell types of a particular tissue. Exemplary adult stem cells include hematopoietic stem cells, neural stem cells, and mesenchymal stem cells.

As used herein, reprogramming is intended to refer to a process that alters or reverses the differentiation status of a somatic cell that is either partially or terminally differentiated. Reprogramming of a somatic cell may be a partial or complete reversion of the differentiation status of the somatic cell. In an exemplary aspect, reprogramming is complete wherein a somatic cell is reprogrammed into an induced pluripotent stem cell. However, reprogramming may be partial, such as reversion into any less differentiated state. For example, reverting a terminally differentiated cell into a cell of a less differentiated state, such as a multipotent cell.

In various aspects of the present invention, nuclear reprogramming factors are genes that induce pluripotency and utilized to reprogram differentiated or semi-differentiated cells to a phenotype that is more primitive than that of the initial cell, such as the phenotype of a pluripotent stem cell. Such genes are utilized with agents that alter microRNA levels or activities in the cell and/or inhibit p21 expression or activity to increase induction efficiency. Such genes and agents are capable of generating a pluripotent stem cell from a somatic cell upon expression of one or more such genes having been integrated into the genome of the somatic cell. As used herein, a gene that induces pluripotency is intended to refer to a gene that is associated with pluripotency and capable of generating a less differentiated cell, such as a pluripotent stem cell from a somatic cell upon integration and expression of the gene. The expression of a pluripotency gene is typically restricted to pluripotent stem cells, and is crucial for the functional identity of pluripotent stem cells.

One of skill in the art would appreciate that agents that alter the level or activity of microRNA in a cell or inhibit p21 expression or activity include a variety of different types of molecules. An agent useful in any of the methods of the invention can be any type of molecule, for example, a polynucleotide, a peptide, a peptidomimetic, peptoids such as vinylogous peptoids, chemical compounds, such as organic molecules or small organic molecules, or the like. Accordingly, in one aspect, an agent for use in the method of the present invention is a polynucleotide, such as an antisense oligonucleotide or RNA molecule. In various aspects, the agent may be a polynucleotide, such as an antisense oligonucleotide or RNA molecule, such as microRNA, dsRNA, siRNA, stRNA, and shRNA. In exemplary aspects, the agent is a microRNA that is introduced into the cell thus increasing the levels and activity of microRNA in the cell and/or inhibiting p21.

MicroRNAs (miRNA) are single-stranded RNA molecules, which regulate gene expression. miRNAs are encoded by genes from whose DNA they are transcribed but miRNAs are not translated into protein; instead each primary transcript (a pri-miRNA) is processed into a short stem-loop structure called a pre-miRNA and finally into a functional miRNA. Mature miRNA molecules are either fully or partially complementary to one or more messenger RNA (mRNA) molecules, and their main function is to down-regulate gene expression. MicroRNAs can be encoded by independent genes, but also be processed (via the enzyme Dicer) from a variety of different RNA species, including introns, 3′ UTRs of mRNAs, long noncoding RNAs, snoRNAs and transposons. As used herein, microRNAs also include “mimic” microRNAs which are intended to mean a microRNA exogenously introduced into a cell that have the same or substantially the same function as their endogenous counterpart. Thus, while one of skill in the art would understand that an agent may be an exogenously introduced RNA, an agent also includes a compound or the like that increase or decrease expression of microRNA in the cell.

In various aspects, the microRNA may be a microRNA included in cluster that exhibits an increase or decrease in activity or expression during induction of an iPSC or differentiation thereof. In one aspect the microRNA may be one or more microRNAs in the miR-17, miR-25, miR-106a, or miR-302b cluster, such as miR-93, miR-106b, or any combination thereof. Induction of miR-17˜92, miR-106b˜25 and miR-106a˜363 clusters are shown to be important for proper reprogramming. Such microRNAs appear to lower the reprogramming barrier during the process and therefore the level of these microRNAs in the cells may be manipulated to improve reprogramming efficiency. MicroRNAs may also be manipulated to direct differentiation of an iPSC since microRNAs are shown to be important regulatory molecules.

Three clusters of microRNAs are identified herein to be induced during iPSC induction and several microRNAs within these clusters have been determined to have the same nucleotide seed region sequences indicating they target to similar mRNAs. It has also been determined that such microRNAs sharing the nucleotide sequence of the same seed region enhance iPSC induction while decreasing p21 expression. Thus in one aspect, the microRNA has a polynucleotide sequence comprising SEQ ID NO: 1,5′-AAGUGC-3′, which has been determined to be conserved between various microRNAs, e.g., those of SEQ ID NOs: 2-11. Thus in a related aspect, the microRNA has the nucleotide sequence of any of SEQ ID NOs: 2-11.

The terms “small interfering RNA” and “siRNA” also are used herein to refer to short interfering RNA or silencing RNA, which are a class of short double-stranded RNA molecules that play a variety of biological roles. Most notably, siRNA is involved in the RNA interference (RNAi) pathway where the siRNA interferes with the expression of a specific gene. In addition to their role in the RNAi pathway, siRNAs also act in RNAi-related pathways (e.g., as an antiviral mechanism or in shaping the chromatin structure of a genome).

Polynucleotides of the present invention, such as antisense oligonucleotides and RNA molecules may be of any suitable length. For example, one of skill in the art would understand what length are suitable for antisense oligonucleotides or RNA molecule to be used to regulate gene expression. Such molecules are typically from about 5 to 100, 5 to 50, 5 to 45, 5 to 40, 5 to 35, 5 to 30, 5 to 25, 5 to 20, or 10 to 20 nucleotides in length. For example the molecule may be about 5, 10, 15, 16, 17, 18, 19, 20, 21, 22, 23, 24, 25, 26, 27, 28, 29, 30, 31, 32, 33, 34, 35, 40, 45 or 50 nucleotides in length. Such polynucleotides may include from at least about 15 to more than about 120 nucleotides, including at least about 16 nucleotides, at least about 17 nucleotides, at least about 18 nucleotides, at least about 19 nucleotides, at least about 20 nucleotides, at least about 21 nucleotides, at least about 22 nucleotides, at least about 23 nucleotides, at least about 24 nucleotides, at least about 25 nucleotides, at least about 26 nucleotides, at least about 27 nucleotides, at least about 28 nucleotides, at least about 29 nucleotides, at least about 30 nucleotides, at least about 35 nucleotides, at least about 40 nucleotides, at least about 45 nucleotides, at least about 50 nucleotides, at least about 55 nucleotides, at least about 60 nucleotides, at least about 65 nucleotides, at least about 70 nucleotides, at least about 75 nucleotides, at least about 80 nucleotides, at least about 85 nucleotides, at least about 90 nucleotides, at least about 95 nucleotides, at least about 100 nucleotides, at least about 110 nucleotides, at least about 120 nucleotides or greater than 120 nucleotides.

The term “polynucleotide” or “nucleotide sequence” or “nucleic acid molecule” is used broadly herein to mean a sequence of two or more deoxyribonucleotides or ribonucleotides that are linked together by a phosphodiester bond. As such, the terms include RNA and DNA, which can be a gene or a portion thereof, a cDNA, a synthetic polydeoxyribonucleic acid sequence, or the like, and can be single stranded or double stranded, as well as a DNA/RNA hybrid. Furthermore, the terms as used herein include naturally occurring nucleic acid molecules, which can be isolated from a cell, as well as synthetic polynucleotides, which can be prepared, for example, by methods of chemical synthesis or by enzymatic methods such as by the polymerase chain reaction (PCR). It should be recognized that the different terms are used only for convenience of discussion so as to distinguish, for example, different components of a composition.

In general, the nucleotides comprising a polynucleotide are naturally occurring deoxyribonucleotides, such as adenine, cytosine, guanine or thymine linked to 2′-deoxyribose, or ribonucleotides such as adenine, cytosine, guanine or uracil linked to ribose. Depending on the use, however, a polynucleotide also can contain nucleotide analogs, including non-naturally occurring synthetic nucleotides or modified naturally occurring nucleotides. Nucleotide analogs are well known in the art and commercially available, as are polynucleotides containing such nucleotide analogs. The covalent bond linking the nucleotides of a polynucleotide generally is a phosphodiester bond. However, depending on the purpose for which the polynucleotide is to be used, the covalent bond also can be any of numerous other bonds, including a thiodiester bond, a phosphorothioate bond, a peptide-like bond or any other bond known to those in the art as useful for linking nucleotides to produce synthetic polynucleotides.

A polynucleotide or oligonucleotide comprising naturally occurring nucleotides and phosphodiester bonds can be chemically synthesized or can be produced using recombinant DNA methods, using an appropriate polynucleotide as a template. In comparison, a polynucleotide comprising nucleotide analogs or covalent bonds other than phosphodiester bonds generally will be chemically synthesized, although an enzyme such as T7 polymerase can incorporate certain types of nucleotide analogs into a polynucleotide and, therefore, can be used to produce such a polynucleotide recombinantly from an appropriate template.

In various embodiments antisense oligonucleotides or RNA molecules include oligonucleotides containing modifications. A variety of modification are known in the art and contemplated for use in the present invention. For example oligonucleotides containing modified backbones or non-natural internucleoside linkages are contemplated. As used herein, oligonucleotides having modified backbones include those that retain a phosphorus atom in the backbone and those that do not have a phosphorus atom in the backbone. For the purposes of this specification, and as sometimes referenced in the art, modified oligonucleotides that do not have a phosphorus atom in their internucleoside backbone can also be considered to be oligonucleosides.

In various aspects modified oligonucleotide backbones include, for example, phosphorothioates, chiral phosphorothioates, phosphorodithioates, phosphotriesters, aminoalkylphosphotriesters, methyl and other alkyl phosphonates including 3′-alkylene phosphonates, 5′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, selenophosphates and borano-phosphates having normal 3′-5′ linkages, 2′-5′ linked analogs of these, and those having inverted polarity wherein one or more internucleotide linkages is a 3′ to 3′, 5′ to 5′ or 2′ to 2′ linkage. Certain oligonucleotides having inverted polarity comprise a single 3′ to 3′ linkage at the 3′-most internucleotide linkage i.e. a single inverted nucleoside residue which may be abasic (the nucleobase is missing or has a hydroxyl group in place thereof). Various salts, mixed salts and free acid forms are also included.

In various aspects modified oligonucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; riboacetyl backbones; alkene containing backbones; sulfamate backbones; methyleneimino and methylenehydrazino backbones; sulfonate and sulfonamide backbones; amide backbones; and others having mixed N, O, S and CH₂ component parts.

In various aspects, oligonucleotide mimetics, both the sugar and the internucleoside linkage, i.e., the backbone, of the nucleotide units are replaced with novel groups. The base units are maintained for hybridization with an appropriate nucleic acid target compound. One such oligomeric compound, an oligonucleotide mimetic that has been shown to have excellent hybridization properties, is referred to as a peptide nucleic acid (PNA). In PNA compounds, the sugar-backbone of an oligonucleotide is replaced with an amide containing backbone, in particular an aminoethylglycine backbone. The nucleobases are retained and are bound directly or indirectly to aza nitrogen atoms of the amide portion of the backbone. In various aspects, oligonucleotides may include phosphorothioate backbones and oligonucleosides with heteroatom backbones. Modified oligonucleotides may also contain one or more substituted sugar moieties. In some embodiments oligonucleotides comprise one of the following at the 2′ position: OH; F; O—, S—, or N-alkyl; O—, S—, or N-alkenyl; O—, S- or N-alkynyl; or O-alkyl-O-alkyl, wherein the alkyl, alkenyl and alkynyl may be substituted or unsubstituted C₁ to C₁₀ alkyl or C₂ to C₁₀ alkenyl and alkynyl. Particularly preferred are O[(CH₂)ₙO]ₘCH₃, O(CH₂)ₙOCH₃, O(CH₂)ₙNH₂, O(CH₂)ₙCH₃, O(CH₂)ₙONH₂ and O(CH₂)ₙON[(CH₂)ₙCH₃)]₂, where n and m are from 1 to about 10. Other preferred oligonucleotides comprise one of the following at the 2′ position: C₁ to C₁₀ lower alkyl, substituted lower alkyl, alkenyl, alkynyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N3, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. Another modification includes 2′-methoxyethoxy(2′OCH₂CH₂OCH₃, also known as 2′-O-(2-methoxyethyl) or 2′-MOE).

In related aspects, the present invention includes use of Locked Nucleic Acids (LNAs) to generate antisense nucleic acids having enhanced affinity and specificity for the target polynucleotide. LNAs are nucleic acid in which the 2′-hydroxyl group is linked to the 3′ or 4′ carbon atom of the sugar ring thereby forming a bicyclic sugar moiety. The linkage is preferably a methelyne (—CH₂—)ₙ group bridging the 2′ oxygen atom and the 4′ carbon atom wherein n is 1 or 2.

Other modifications include 2′-methoxy(2′-O—CH₃), 2′-aminopropoxy(2′-OCH₂CH₂CH₂NH₂), 2′-allyl(2′-CH—CH—CH₂), 2′-O-allyl(2′-O—CH₂—CH—CH₂), 2′-fluoro (2′-F), 2′-amino, 2′-thio, 2′-Omethyl, 2′-methoxymethyl, 2′-propyl, and the like. The 2′-modification may be in the arabino (up) position or ribo (down) position. A preferred 2′-arabino modification is 2′-F. Similar modifications may also be made at other positions on the oligonucleotide, particularly the 3′ position of the sugar on the 3′ terminal nucleotide or in 2′-5′ linked oligonucleotides and the 5′ position of 5′ terminal nucleotide. Oligonucleotides may also have sugar mimetics such as cyclobutyl moieties in place of the pentofuranosyl sugar.

Oligonucleotides may also include nucleobase modifications or substitutions. As used herein, “unmodified” or “natural” nucleobases include the purine bases adenine (A) and guanine (G), and the pyrimidine bases thymine (T), cytosine (C) and uracil (U). Modified nucleobases include other synthetic and natural nucleobases such as 5-methylcytosine, 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-amino-adenine, 8-azaguanine and 8-azaadenine, 7-deazaguanine and 7-deazaadenine and 3-deazaguanine and 3-deazaadenine. Further modified nucleobases include tricyclic pyrimidines such as phenoxazine cytidine (1H-pyrimido[5,4-b][1,4]benzoxazi-n-2(3H)-one), phenothiazine cytidine (1H-pyrimido[5,4-b][1,4]benzothiazin-2(3H)-one), G-clamps such as a substituted phenoxazine cytidine (e.g. 9-(2-aminoethoxy)-H-pyrimido[5,4-b][1,4]benzoxazin-2(3H)-one), carbazole cytidine (2H-pyrimido[4,5-b]indol-2-one), pyridoindole cytidine (H-pyrimido[3′,2′:4,5]pyrrolo[2,3-d]pyrimidin-2-one). Modified nucleobases may also include those in which the purine or pyrimidine base is replaced with other heterocycles, for example 7-deaza-adenine, 7-deazaguanosine, 2-aminopyridine and 2-pyridone. Further nucleobases are known in the art. Certain of these nucleobases are particularly useful for increasing the binding affinity of the oligomeric compounds described herein. These include 5-substituted pyrimidines, 6-azapyrimidines and N-2, N-6 and O-6 substituted purines, including 2-aminopropyladenine, 5-propynyluracil and 5-propynylcytosine. 5-methylcytosine substitutions have been shown to increase nucleic acid duplex stability by 0.6-1.2 C and are presently preferred base substitutions, even more particularly when combined with 2′-O-methoxyethyl sugar modifications.

Another modification of the antisense oligonucleotides described herein involves chemically linking to the oligonucleotide one or more moieties or conjugates which enhance the activity, cellular distribution or cellular uptake of the oligonucleotide. The antisense oligonucleotides can include conjugate groups covalently bound to functional groups such as primary or secondary hydroxyl groups. Conjugate groups include intercalators, reporter molecules, polyamines, polyamides, polyethylene glycols, polyethers, groups that enhance the pharmacodynamic properties of oligomers, and groups that enhance the pharmacokinetic properties of oligomers. Typical conjugates groups include cholesterols, lipids, phospholipids, biotin, phenazine, folate, phenanthridine, anthraquinone, acridine, fluoresceins, rhodamines, coumarins, and dyes. Groups that enhance the pharmacodynamic properties, in the context of this invention, include groups that improve oligomer uptake, enhance oligomer resistance to degradation, and/or strengthen sequence-specific hybridization with RNA. Groups that enhance the pharmacokinetic properties, in the context of this invention, include groups that improve oligomer uptake, distribution, metabolism or excretion. Conjugate moieties include but are not limited to lipid moieties such as a cholesterol moiety, cholic acid, a thioether, e.g., hexyl-5-tritylthiol, a thiocholesterol, an aliphatic chain, e.g., dodecandiol or undecyl residues, a phospholipid, e.g., dihexadecyl-rac-glycerol or triethylammonium 1,2-di-O-hexadecyl-rac-glycero-3-H-phosphonate, a polyamine or a polyethylene glycol chain, or adamantane acetic acid, a palmityl moiety, or an octadecylamine or hexylaminocarbonyloxycholesterol moiety.

Several genes have been found to be associated with pluripotency and suitable for use with the present invention as reprogramming factors. Such genes are known in the art and include, by way of example, SOX family genes (SOX1, SOX2, SOX3, SOX15, SOX18), KLF family genes (KLF1, KLF2, KLF4, KLF5), MYC family genes (C-MYC, L-MYC, N-MYC), SALL4, OCT4, NANOG, LIN28, STELLA, NOBOX or a STAT family gene. STAT family members may include for example STAT1, STAT2, STAT3, STAT4, STAT5 (STAT5A and STAT5B), and STAT6. While in some instances, use of only one gene to induce pluripotency may be possible, in general, expression of more than one gene is required to induce pluripotency. For example, two, three, four or more genes may be simultaneously integrated into the somatic cell genome as a polycistronic construct to allow simultaneous expression of such genes. In an exemplary aspect, four genes are utilized to induce pluripotency including OCT4, SOX2, KLF4 and C-MYC. Additional genes known as reprogramming factors suitable for use with the present invention are disclosed in U.S. patent application Ser. No. 10/997,146 and U.S. patent application Ser. No. 12/289,873, incorporated herein by reference.

All of these genes commonly exist in mammals, including human, and thus homologues from any mammals may be used in the present invention, such as genes derived from mammals including, but not limited to mouse, rat, bovine, ovine, horse, and ape. Further, in addition to wild-type gene products, mutant gene products including substitution, insertion, and/or deletion of several (e.g., 1 to 10, 1 to 6, 1 to 4, 1 to 3, and 1 or 2) amino acids and having similar function to that of the wild-type gene products can also be used. Furthermore, the combinations of factors are not limited to the use of wild-type genes or gene products. For example, Myc chimeras or other Myc variants can be used instead of wild-type Myc.

The present invention is not limited to any particular combination of nuclear reprogramming factors. As discussed herein a nuclear reprogramming factor may comprise one or more gene products. The nuclear reprogramming factor may also comprise a combination of gene products as discussed herein. Each nuclear reprogramming factor may be used alone or in combination with other nuclear reprogramming factors as disclosed herein. Further, nuclear reprogramming factors of the present invention can be identified by screening methods, for example, as discussed in U.S. patent application Ser. No. 10/997,146, incorporated herein by reference. Additionally, the nuclear reprogramming factor of the present invention may contain one or more factors relating to differentiation, development, proliferation or the like and factors having other physiological activities, as well as other gene products which can function as a nuclear reprogramming factor.

The nuclear reprogramming factor may comprise a protein or peptide. The protein may be produced from a gene as discussed herein, or alternatively, in the form of a fusion gene product of the protein with another protein, peptide or the like. The protein or peptide may be a fluorescent protein and/or a fusion protein. For example, a fusion protein with green fluorescence protein (GFP) or a fusion gene product with a peptide such as a histidine tag can also be used. Further, by preparing and using a fusion protein with the TAT peptide derived from the virus HIV, intracellular uptake of the nuclear reprogramming factor through cell membranes can be promoted, thereby enabling induction of reprogramming only by adding the fusion protein to a medium thus avoiding complicated operations such as gene transduction. Since preparation methods of such fusion gene products are well known to those skilled in the art, skilled artisans can easily design and prepare an appropriate fusion gene product depending on the purpose.

As discussed herein, an iPSC may be induced by contacting a somatic cell with a nuclear reprogramming factor in combination with an agent that alters microRNA levels or activity in the cell and/or an inhibitor of p21. As would be appreciated by one of skill in the art, delivery to the somatic cell may be performed by any suitable method known in the art. In one aspect, the nuclear reprogramming factor may be introduced into a cell with a recombinant vector comprising a gene encoding the nuclear reprogramming factor. Similarly, the agents that alter microRNA may be introduced into a cell with a recombinant vector comprising a polynucleotide encoding an RNA molecule, such as a microRNA, shRNA, antisense oligonucleotide and the like. Similarly, the inhibitors of p21 may be introduced into a cell with a recombinant vector comprising a polynucleotide encoding a peptide inhibitor or RNA molecule, such as a microRNA, shRNA, antisense oligonucleotide and the like. Accordingly, the cell can express the nuclear reprogramming factor expressed as a product of a gene contained in the recombinant vector, as well as expressing the agent or p21 inhibitor as a product of a polynucleotide contained in the recombinant vector thereby inducing reprogramming of a differentiated cell at an increased efficiency rate as compare to use of the nuclear reprogramming factor alone.

The nucleic acid construct of the present invention, such as recombinant vectors may be introduced into a cell using a variety of well known techniques, such as non-viral based transfection of the cell. In an exemplary aspect the construct is incorporated into a vector and introduced into the cell to allow expression of the construct. Introduction into the cell may be performed by any viral or non-viral based transfection known in the art, such as, but not limited to electroporation, calcium phosphate mediated transfer, nucleofection, sonoporation, heat shock, magnetofection, liposome mediated transfer, microinjection, microprojectile mediated transfer (nanoparticles), cationic polymer mediated transfer (DEAE-dextran, polyethylenimine, polyethylene glycol (PEG) and the like) or cell fusion. Other methods of transfection include proprietary transfection reagents such as Lipofectamine™, Dojindo Hilymax™, Fugene™, jetPEI™, Effectene™ and DreamFect™.

In other aspects, contacting the somatic cell during induction with a nuclear reprogramming factor in combination with an agent that alters microRNA levels or activity in the cell and/or an inhibitor of p21 may be performed by any method known in the art. For example, direct delivery of proteins, RNA molecules and the like across the cell membrane.

Use of a nuclear reprogramming factor in combination with an agent that alters microRNA levels or activity in the cell and/or an inhibitor of p21 increase the induction efficiency as compared to use of a reprogramming factor alone. In various aspects, induction efficiency may be increased by 10, 20, 30, 40, 50, 60, 70, 80, 90, 100, 150, 200, 300, 400 or ever 500 percent as compared with convention methods. For example, induction efficiency may be as high as 1, 2, 3, 4, 5, 6, 7, 8, 9, 10, 15, 20, 25 or 50 percent (e.g., percent of induced cells as compared with total number of starting somatic cells).

During the induction process, the somatic cell may be contacted with the nuclear reprogramming factor simultaneously or before the cell is contact with the agent that alters microRNA levels or activity in the cell and/or the inhibitor of p21. In various aspects, the somatic cell is contacted with the reprogramming factor about 1, 2, 3, 4, 5, 7, 8, 9, 10, 11, 12, 13, 14 or more days before the cell is contacted with any other agent or inhibitor. In an exemplary aspect, the somatic cell is contacted with the reprogramming factor about 1, 2, 3, 4 or 5 days before the cell is contacted with any other agent or inhibitor.

Further analysis may be performed to assess the pluripotency characteristics of a reprogrammed cell. The cells may be analyzed for different growth characteristics and embryonic stem cell like morphology. For example, cells may be differentiated in vitro by adding certain growth factors known to drive differentiation into specific cell types. Reprogrammed cells capable of forming only a few cell types of the body are multipotent, while reprogrammed cells capable of forming any cell type of the body are pluripotent.

Expression profiling of reprogrammed somatic cells to assess their pluripotency characteristics may also be conducted. Expression of individual genes associated with pluripotency may also be examined. Additionally, expression of embryonic stem cell surface markers may be analyzed. Detection and analysis of a variety of genes known in the art to be associated with pluripotent stem cells may include analysis of genes such as, but not limited to OCT4, NANOG, SALL4, SSEA-1, SSEA-3, SSEA-4, TRA-1-60, TRA-1-81, or a combination thereof. iPS cells may express any number of pluripotent cell markers, including: alkaline phosphatase (AP); ABCG2; stage specific embryonic antigen-1 (SSEA-1); SSEA-3; SSEA-4; TRA-1-60; TRA-1-81; Tra-2-49/6E; ERas/ECAT5, E-cadherin; β-tubulin III; α-smooth muscle actin (α-SMA); fibroblast growth factor 4 (FGF4), Cripto, Dax1; zinc finger protein 296 (Zfp296); N-acetyltransferase-1 (Nat1); ES cell associated transcript 1 (ECAT1); ESG1/DPPA5/ECAT2; ECAT3; ECAT6; ECAT7; ECAT8; ECAT9; ECAT10; ECAT15-1; ECAT15-2; Fth117; Sal14; undifferentiated embryonic cell transcription factor (Utf1); Rex1; p53; G3PDH; telomerase, including TERT; silent X chromosome genes; Dnmt3a; Dnmt3b; TRIM28; F-box containing protein 15 (Fbx15); Nanog/ECAT4; Oct3/4; Sox2; Klf4; c-Myc; Esrrb; TDGF1; GABRB3; Zfp42, FoxD3; GDF3; CYP25A1; developmental pluripotency-associated 2 (DPPA2); T-cell lymphoma breakpoint 1 (Tcl1); DPPA3/Stella; DPPA4; as well as other general markers for Pluripotency, for example any genes used during induction to reprogram the cell. IPS cells can also be characterized by the down-regulation of markers characteristic of the differentiated cell from which the iPS cell is induced.

The invention further provides iPS cells produced using the methods described herein, as well as populations of such cells. The reprogrammed cells of the present invention, capable of differentiation into a variety of cell types, have a variety of applications and therapeutic uses. The basic properties of stem cells, the capability to infinitely self-renew and the ability to differentiate into every cell type in the body make them ideal for therapeutic uses.

Accordingly, in one aspect the present invention further provides a method of treatment or prevention of a disorder and/or condition in a subject using induced pluripotent stem cells generated using the methods described herein. The method includes obtaining a somatic cell from a subject and reprogramming the somatic cell into an induced pluripotent stem (iPS) cell using the methods described herein. The cell is then cultured under suitable conditions to differentiate the cell into a desired cell type suitable for treating the condition. The differentiated cell may then be introducing into the subject to treat or prevent the condition.

In one aspect, the iPS cells produced using the methods described herein, as well as populations of such cells may be differentiated in vitro by treating or contacting the cells with agents that alter microRNA levels or activities in the cells. Since microRNAs have been identified as key regulators in iPSC induction, it is expected that manipulation of individual microRNAs or populations of microRNAs may be used in directing differentiation of such iPSCs. Such treatment may be used in combination with growth factors or other agents and stimuli commonly known in the art to drive differentiation into specific cell types.

One advantage of the present invention is that it provides an essentially limitless supply of isogenic or synegenic human cells suitable for transplantation. The iPS cells are tailored specifically to the patient, avoiding immune rejection. Therefore, it will obviate the significant problem associated with current transplantation methods, such as, rejection of the transplanted tissue which may occur because of host versus graft or graft versus host rejection. Several kinds of iPS cells or fully differentiated somatic cells prepared from iPS cells from somatic cells derived from healthy humans can be stored in an iPS cell bank as a library of cells, and one kind or more kinds of the iPS cells in the library can be used for preparation of somatic cells, tissues, or organs that are free of rejection by a patient to be subjected to stem cell therapy.

The iPS cells of the present invention may be differentiated into a number of different cell types to treat a variety of disorders by methods known in the art. For example, iPS cells may be induced to differentiate into hematopoetic stem cells, muscle cells, cardiac muscle cells, liver cells, cartilage cells, epithelial cells, urinary tract cells, neuronal cells, and the like. The differentiated cells may then be transplanted back into the patient's body to prevent or treat a condition. Thus, the methods of the present invention may be used to treat a subject having a myocardial infarction, congestive heart failure, stroke, ischemia, peripheral vascular disease, alcoholic liver disease, cirrhosis, Parkinson's disease, Alzheimer's disease, diabetes, cancer, arthritis, wound healing, immunodeficiency, aplastic anemia, anemia, Huntington's disease, amyotrophic lateral sclerosis (ALS), lysosomal storage diseases, multiple sclerosis, spinal cord injuries, genetic disorders, and similar diseases, where an increase or replacement of a particular cell type/tissue or cellular de-differentiation is desirable.

In various embodiments, the method increases the number of cells of the tissue or organ by at least about 5%, 10%, 25%, 50%, 75% or more compared to a corresponding untreated control tissue or organ. In yet another embodiment, the method increases the biological activity of the tissue or organ by at least about 5%, 10%, 25%, 50%, 75% or more compared to a corresponding untreated control tissue or organ. In yet another embodiment, the method increases blood vessel formation in the tissue or organ by at least about 5%, 10%, 25%, 50%, 75% or more compared to a corresponding untreated control tissue or organ. In yet another embodiment, the cell is administered directly to a subject at a site where an increase in cell number is desired.

The present invention further provides a method for evaluating a physiological function or toxicity of an agent, compound, a medicament, a poison or the like by using various cells obtained by the methods described herein.

Somatic cells can be reprogrammed to an ES-like state to create induced pluripotent stem cells (iPSCs) by ectopic expression of four transcription factors, Oct4, Sox2, Klf4 and cMyc. The present invention provides that cellular microRNAs regulate iPSC generation. Knock-down of key microRNA pathway proteins can result in significant decreases in reprogramming efficiency. Three microRNA clusters, miR-17˜92, 106b˜25 and 106a˜363, are shown to be highly induced during early reprogramming stages. Several microRNAs, including miR-93 and miR-106b, which have very similar seed regions, greatly enhanced iPSC induction, and inhibiting these microRNAs significantly decreased reprogramming efficiency. Moreover, miR-iPSC clones can reach the fully reprogrammed state. The present invention provides that Tgfbr2 and p21 are directly targeted by these microRNAs and that siRNA knock-down of both genes indeed enhanced iPSC induction. The present invention also provides that miR-93 and its family members directly target TGF-β receptor II to enhance iPSC reprogramming. The present invention provides that microRNAs function in the reprogramming process and that iPSC induction efficiency can be greatly enhanced by modulating microRNA levels in cells.

Although induced pluripotent stem cells (iPSCs) hold great promise for customized-regenerative medicine, the molecular basis of reprogramming is largely unknown. Overcoming barriers that maintain cell identities is a critical step in the reprogramming of differentiated cells. Since microRNAs (miRNAs) modulate target genes tissue-specifically, the invention provides that distinct mouse embryonic fibroblast (MEF)-enriched miRNAs post-transcriptionally modulate proteins that function as reprogramming barriers. Inhibiting these miRNAs should influence cell signaling to lower those barriers. The invention provides that depleting miR-21 and miR-29a enhances reprogramming efficiency in MEFs. The invention provides that p53 and ERK1/2 pathways are regulated by miR-21 and miR-29a and function in reprogramming. The invention further provides that c-Myc enhances reprogramming partly by repressing MEF-enriched miRNAs, such as miR-21 and miR-29a. The invention provides miRNA function in regulating multiple signaling networks involved in iPSC reprogramming.

C-Myc, one of the four reprogramming factors (4F: Oct3/4, Sox2, Klf4, and c-Myc), plays crucial roles in cell proliferation and tumor development. C-Myc is a key regulator of cytostasis and apoptosis through repression of the cyclin-dependent kinase (CDK) inhibitor p21^{Cip1}. By abrogating Miz-1 function and suppressing p15^{INK4b}, c-Myc plays a critical role in the immortalization of primary cells. Many transcriptional functions of c-Myc require cooperation with Max or Miz-1. As a proto-oncogene c-Myc greatly enhances reprogramming efficiency, although it is dispensable for reprogramming. Therefore, defining molecular pathways downstream of c-Myc during reprogramming can enhance therapeutic application of iPS cells, without compromising reprogramming efficiency.

Oct4-GFP mouse embryonic fibroblasts (MEFs) are derived from mice carrying an IRES-EGFP fusion cassette downstream of the stop codon of pou5f1 (Jackson lab, Stock#008214) at D13.5. These MEFs are cultured in DMEM (Invitrogen, 11995-065) with 10% FBS (Invitrogen) plus glutamine and NEAA. For iPSC induction, only MEFs with passage of 0 to 4 are used.

C-Myc reportedly acts to maintain ES cell renewal in part by regulating microRNA (miRNA) expression. MicroRNAs are 22-nucleotide non-coding small RNAs, which are loaded into RNA-induced silencing complex (RISC) to exert a global gene-silencing function. Expression of miR-141, miR-200, and miR-429 is induced by c-Myc in ES cells to antagonize differentiation. C-Myc also promotes tumorigenesis by upregulating the miR-17-92 microRNA cluster or by repressing known tumor suppressors, such as the let-7 family, miR-15a/16-1, the miR-29 family, and miR-34a.

Overcoming barriers securing somatic cell identity and mediated by factors such as Ink4-Arf, p53, and p21 is a rate-limiting step in reprogramming. Since miRNAs modulate target genes tissue-specifically, the invention provides that distinct MEF miRNAs post-transcriptionally modulate proteins that function as reprogramming regulators. Inhibiting these miRNAs can influence cell signaling to lower those barriers.

The invention provides that depleting the abundant miRNAs miR-21 and miR-29a in MEFs enhances reprogramming efficiency by ˜2.1- to 2.8-fold. The invention also provides that c-Myc represses miRNAs miR-21 and miR-29a to enhance reprogramming of MEFs. The invention further provides that miR-21 and miR-29a regulate p53 and ERK1/2 pathways by indirectly down-regulating p53 levels and ERK1/2 phosphorylation during the reprogramming process.

The following examples are provided to further illustrate the embodiments of the present invention, but are not intended to limit the scope of the invention. While they are typical of those that might be used, other procedures, methodologies, or techniques known to those skilled in the art may alternatively be used.

### Example 1

### Cell Culture, Vectors, and Virus Transduction

Oct4-GFP mouse embryonic fibroblasts (MEFs) are derived from mice carrying an IRES-EGFP fusion cassette downstream of the stop codon of pou5f1 (Jackson lab, Stock#008214) at D13.5. These MEFs are cultured in DMEM (Invitrogen, 11995-065) with 10% FBS (Invitrogen) plus glutamine and NEAA. For iPSC induction, only MEFs with passage of 0 to 4 are used.

The plasmids pMXs-Oct4, Sox2, Klf4 and cMyc are purchased from Addgene. The plasmid pMX-HA-p21 is generated by inserting N-terminal tagged-p21 into EcoRI site of pMX vector. The clones of pLKO-shRNAs are purchased from Open-Biosystems.

To generate retrovirus, PLAT-E cells are seeded in 10 cm plates, and 9 μg of each factors are transfected next day using Lipofectamine™ (Invitrogen, 18324-012) and PLUS™ (Invitrogen, 11514-015). Viruses are harvested and combined 2 days later. For iPSC induction, MEFs are seeded in 12-well plates and transduced with four factor virus the next day with 4 μg/ml Polybrene. One day after transduction, medium is changed to fresh MEF medium and 3 days later changed to mES culture medium supplemented with LIF (Millipore, ESG1107). GFP+ colonies are picked up from day 14 post transduction and successfully expanded clones were cultured in DMEM with 15% FBS (Hyclone) plus LIF, thioglycerol, glutamine and NEAA. Irradiated CF1 MEFs are used as the feeder layer for culture of mES and derived iPSC clones.

To generate shRNA lentivirus, shRNA lentivirus vectors are cotransfected into 293FT cells together with the pPACKH1 packaging system (SBI, Cat#LV500A-1). Lentiviruses are harvested at day 2 after transfection and centrifuged at 4,000 rpm for 5 min at room temperature. To produce virus, 4 μg of pLKO or pGIPZ vectors and 10 μg of packaging mix were transfected into 293FT cells (Invitrogen) in 10 cm tissue culture plates. 2 days after transfection, virus containing supernatant was harvested and used for further transduction with 4 μg/μl polybrene. ShRNA virus is added together with 4 factor virus at a volume ratio of 1:1:1:1:1.

MicroRNAs, siRNAs and transfection of MEFs are performed as follows: MicroRNA mimics and inhibitors, siRNAs are purchased from Dharmacon. To transfect MEFs, microRNAs mimic are diluted in Opti-MEM (Invitrogen, 11058-021) to desired final concentration. Lipofectamine™ 2000 (Invitrogen, 11668-019) is then added into the mix at 2 μl/well and incubated 20 min at room temperature. For 12-well transfection, 80 μl miR mixture is added to each well with 320 μl of Opti-MEM. Three hours later, 0.8 ml of virus mixture (for iPSC) or fresh medium is added to each well and the medium is changed to fresh MEF medium the next day.

Western blotting is performed as follows: Total cell lysates are prepared using MPER buffer (PIERCE, 78503) on ice for 20 min and cleared by centrifuging at 13,000 rpm for 10 min. An equal volume of lysates is loaded in 10% SDS-PAGE gels, and proteins are transferred to PVDF membrane (Bio-Rad, 1620177) using a semi-dry system (Bio-Rad). The PVDF membranes are then blocked with 5% milk in TBST for at least 1 hour at room temperature or overnight at 4° C.

Antibodies used include anti-p21 (BD, 556430), anti-mNanog (R&D, AF2729), anti-h/mSSEA1 (R&D, MAB2156), anti-HA (Roche, 11867423001), anti-mAgo2 (Wako, 01422023), anti-Dicer (Abeam, ab13502), anti-Drosha (Abeam, ab12286), anti-Actin (Thermo, MS1295P0), anti-AFP (Abeam, ab7751), anti-β tubulin III (R&D systems, MAB 1368), anti-α actinin (Sigma, A7811).

mRNA and microRNA RT and quantitative PCR are preformed as follows: Total RNAs are extracted by Trizol method (Invitrogen). After extraction, 1 μg total RNA is used for RT by Superscript II™ (Invitrogen). Quantitative PCR is performed by using Roche LightCycler480 II™ and Sybrgreen mixture from Abgene (Ab-4166). Primers for mouse Ago2, Dicer, Drosha, Graph, and p21 are listed in Table 1 below. Other primers have been described in Takahashi, K. and S. Yamanaka (2006) Cell 126(4): 663-76.

For microRNA quantitative analysis, total RNA is extracted using the method described above. After extraction, 1.5˜3 μg of total RNA is used for microRNA reverse transcription using QuantiMir™ kit following manufacturer protocol (SBI, RA420A-1). RT products then are used for quantitative PCR using mature microRNA sequence as forward primer and the universal primer provided with the kit.

Immunostaining is performed as follows: Cells are washed twice with PBS and fixed with 4% paraformaldehyde at room temperature for 20 min. Fixed cells are permeablized with 0.1% Triton X-100 for 5 min. The cells are then blocked in 5% BSA in PBS containing 0.1% Triton X-100 for 1 hour at room temperature. Primary antibody is diluted from 1:100 to 1:400 in 2.5% BSA PBS containing 0.1% Triton X-100 according to manufacturer suggestion. The cells are then stained with primary antibody for 1 hour and then washed three times with PBS. Secondary antibody is diluted at 1:400 and the cells are stained for 45 min at room temperature.

Embryoid body (EB) formation and differentiation assays are performed as follows: iPS cells are trypsinized into single cell suspension and hanging drop method is used to generate embryonic bodies. For each drop, 4000 iPS cells in 20 μl EB differentiation medium are used. EBs are cultured in hanging drop for 3 days before reseeded into gelatin coated plates. After reseeding, cells are further cultured until day 14 when apparent beating areas could be identified.

TABLE 1
Primers for qPCR analysis

| | | |
|---|---|---|
| mmuAgo2 | Forward | 5′-gcgtcaacaacatcctgct-3′ |
| | Reverse | 5′-ctcccaggaagatgacaggt-3′ |
| mmuDrosha | Forward | 5′-cgtctctagaaaggtcctacaagaa-3′ |
| | Reverse | 5′-ggctcaggagcaactggtaa-3′ |
| mmuDicer1 | Forward | 5′-gggctgtatgagagattgctgatg-3′ |
| | Reverse | 5′-cacggcagtctgagaggatttg-3′ |
| mmuP21 | Forward | 5′-tccacagcgatatccagaca-3′ |
| | Reverse | 5′-ggacatcaccaggattggac-3′ |
| mmuGAPDH | Forward | 5′-atcaagaaggtggtgaagcggaa-3′ |
| | Reverse | 5′-tggaagagtgggagttgctgttga-3′ |

Promoter methylation analysis is performed as follows: CpG methylation of Nanog and Pou5f1 promoter is analyzed following the same procedure described previously (Takahashi, K. and S. Yamanaka (2006) Cell 126(4): 663-76). Briefly, genomic DNA of derived clones is extracted using Qiagen™ kit. 1 μg of DNA is then used for genome modification analysis following manufacturer protocol (EZ DNA Methylation—Direct kit, Zymo Research, D5020). After modification, PCR of selected regions is performed and the products are cloned into pCR2.1-TOPO™ (Invitrogen). Ten clones are sequenced for each gene.

### Example 2

### Teratoma Formation, Chimera Generation, and Microarray Analysis

Teratoma formation and chimera generation are performed as follows: To generate teratomas, iPS cells are trypsinized and resuspended at a concentration of 1×10⁷ cells/ml. Athymus nude mice are first anesthetized with Avertin, and then approximately 150 μl of the cell suspension is injected into each mouse. Mice are checked for tumors every week for 3-4 weeks. Tumors are harvested and fixed in zinc formalin solution for 24 hours at room temperature before paraffin embedding and H&E staining. To test the capacity of derived iPSC clones to contribute to chimeras, iPS cells are injected into C57BL/6J-Tyr^{(C-2J)/J}(albino) blastocysts. Generally, each blastocyst receives 12-18 iPS cells. ICR recipient females are used for embryo transfer. The donor iPS cells are either in agouti or black color.

mRNA microarray analysis is performed as follows: miR-93 and siControl are transfected into MEFs and total RNAs are harvested at 48 hours post transfection. mRNA microarray is carried out by Microarray facility in Sanford-Burnham institute. Gene lists for both potential functional targets (fold change>2, p<0.05) and total targets (fold change<25%, p<0.05) are generated by filtering through volcano maps. Gene lists are then used for ontology analysis using GeneGo software following guidelines from the company.

Dual luciferase assay is performed as follows: 3′UTR of both p21 and Tgfbr2 are cloned into XbaI site of pGL3 control vectors. For each well of 12-well plates, 200 ng of resulted vectors and 50 ng of pRL-TK (renilla luciferase) are transfected into 1×10⁵ Hela cells which are seeded one day before the transfection. 50 nM of microRNAs are used for each treatment and cell lysates are harvested at day 2 post transfection. 20 μl of lysates are then used for dual luciferase assay following manufacturer's protocol (Dual-Luciferase® Reporter Assay System Promega, E1910).

Cell proliferation assay is performed as follows: 3000 MEFs are seeded in each well in 96-well plates and transduced with 4F virus and shRNA lentivirus (or transfected with microRNA inhibitors). Starting from day 1 post transduction/transfection, every two days, the cells are incubated with mES medium containing Celltiter 96 Aqueous one solution (Promega, G3580) for 1 hour in tissue culture incubator. Absorbance at 490 nm is then measured for each well using plate reader and collected data is used to generate relative proliferation curve using signal from day 1 post transduction/transfection as the reference.

### Example 3

### Post-Transcriptional Regulation Pathway Is Involved In Reprogramming Somatic Cells

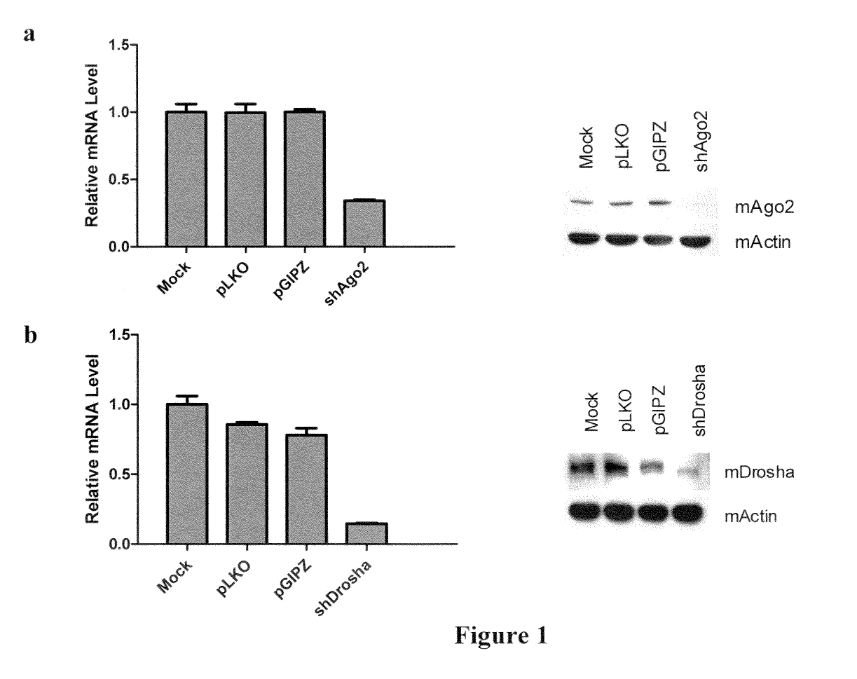

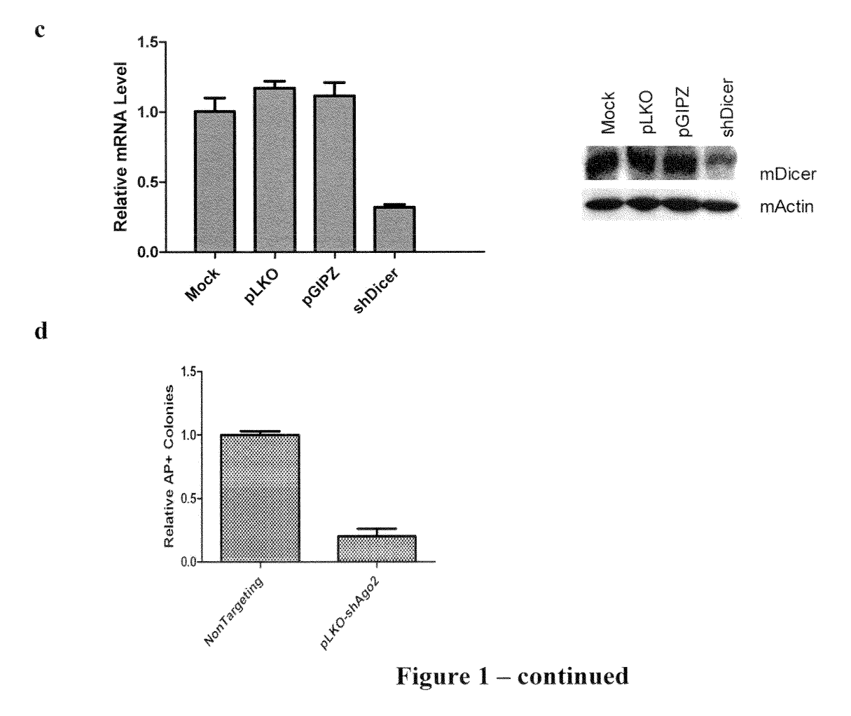

The post-transcriptional regulation pathway was determined to be involved in reprogramming of MEFs to iPS cells. To investigate the role of post-transcriptional gene regulation during iPSC induction, lentiviral shRNA vectors targeting mouse Dicer, Drosha and Ago2 are used for stable knock-down in primary Oct4-GFP MEFs. Knock-down efficiency of these shRNA constructs is verified both by western and RT-qPCR (FIGS. 1a, 1b, and 1c). Approximately 70%-80% of mRNA level knock-down is routinely observed for each shRNA, as well as significant decreases in protein levels.

The shRNAs are then separately used to transduce MEFs along with viruses expressing the four factors OSKM (Oct4, Sox2, Klf4, and cMyc) at a volume ratio of 1:1:1:1:1. After 14 days, the colonies are fixed and stained for alkaline phosphatase (AP) activity, which is a widely used ES cell marker. AP+ colonies are quantified for each treatment and knock-down of key RNAi machinery proteins Dicer, Drosha and Ago2 results in a dramatic decrease of AP+ colonies as compared with pLKO and pGIPZ controls. Similar results are observed by using OSK (three factors 3F) transduction.

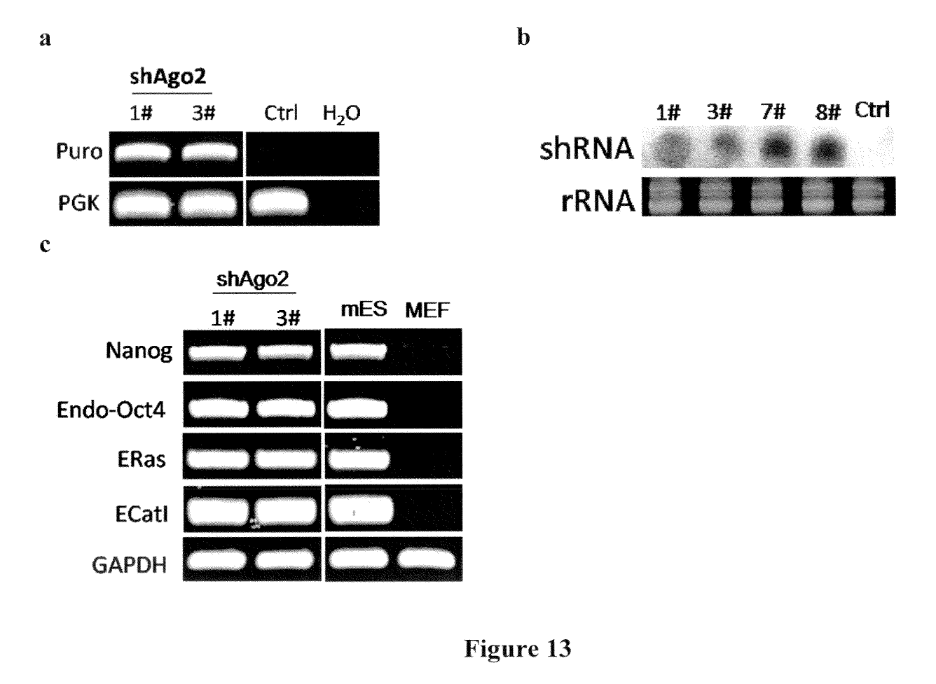

Both GFP+ and AP+ colony quantification verified that knocking down Ago2 dramatically decreases reprogramming efficiency while proliferation of transduced fibroblasts are not affected (FIGS. 1d, 1e, and 1f). Despite the decrease in reprogramming efficiency upon Ago2 knockdown, some GFP+ colonies in shAgo2 are infected MEFs and further characterization determined that these colonies are positive for shRNA integration where shRNAs are actively expressed (FIGS. 13a and 13b). These cells also express all the tested ES-specific markers and have turned on the endogenous Oct4 locus (FIG. 13c). These data strongly suggested that post-transcriptional regulation, especially microRNAs, play a crucial role in the reprogramming process.

### Example 4

### MicroRNA Clusters Are Induced During Reprogramming Of Somatic Cells

MicroRNA miR-17, 25, 106a and 302b clusters are determined to be induced during the early stage of reprogramming. Since the four transcription factors induce a lot of gene expression changes during iPSC induction, it is deduced that some ES specific microRNAs may be induced by these factors, which could help for MEFs to be successfully reprogrammed. Recent publication regarding ES-specific microRNA enhancing iPSC induction also supports the hypothesis, although the reported microRNAs were not found to be expressed until very late stage of reprogramming. By analyzing published results, 9 microRNA clusters determined to be highly expressed in mouse ES cells, are chosen for analysis and shown in Table 2.

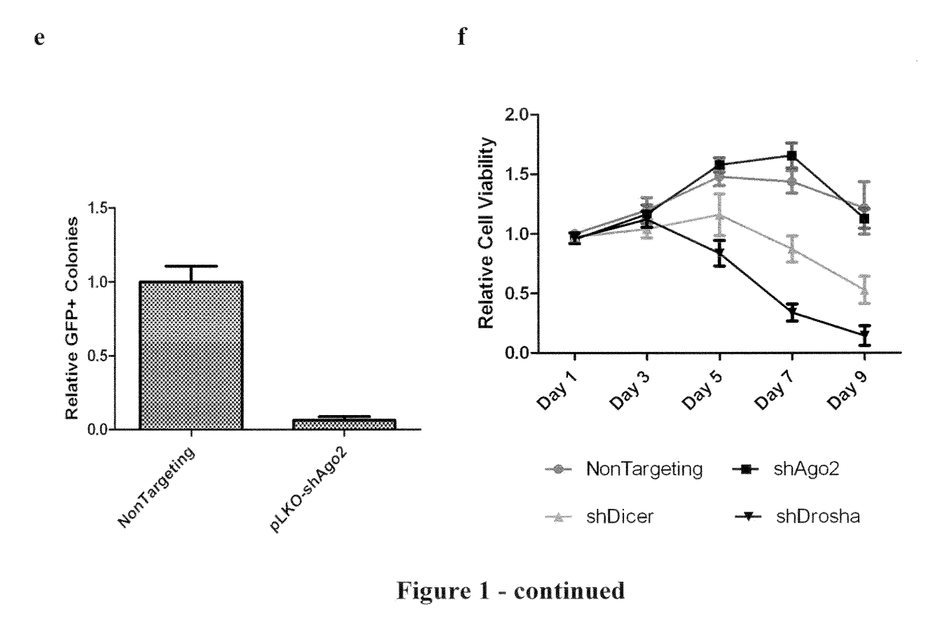

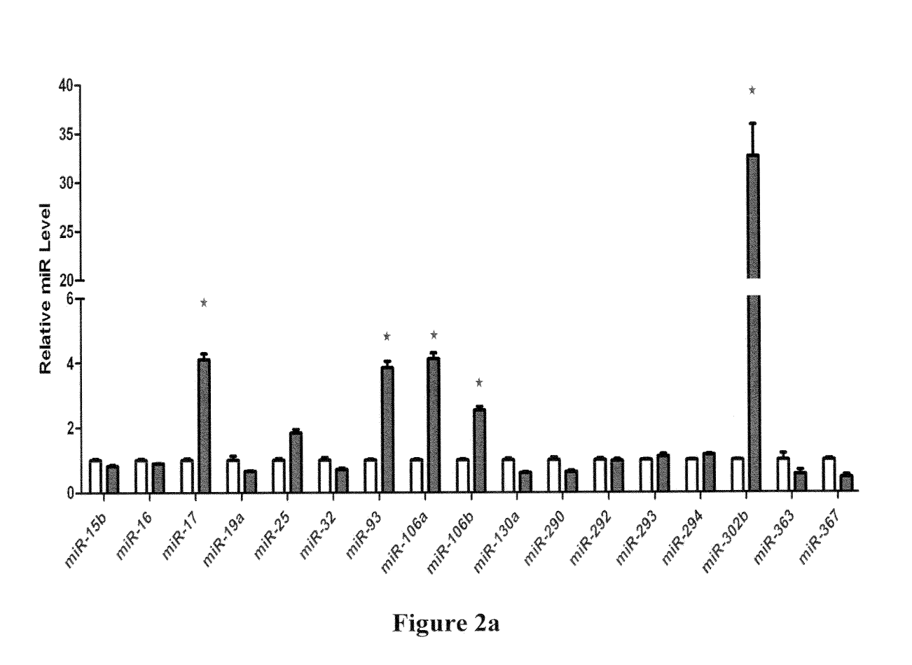

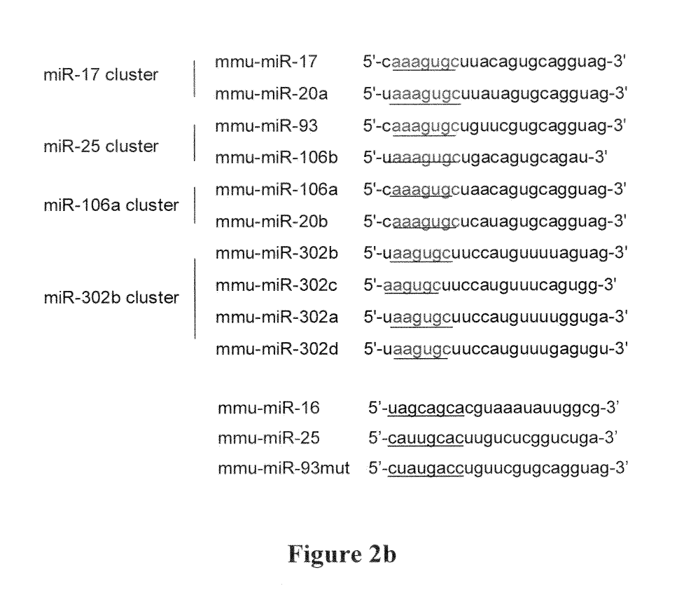

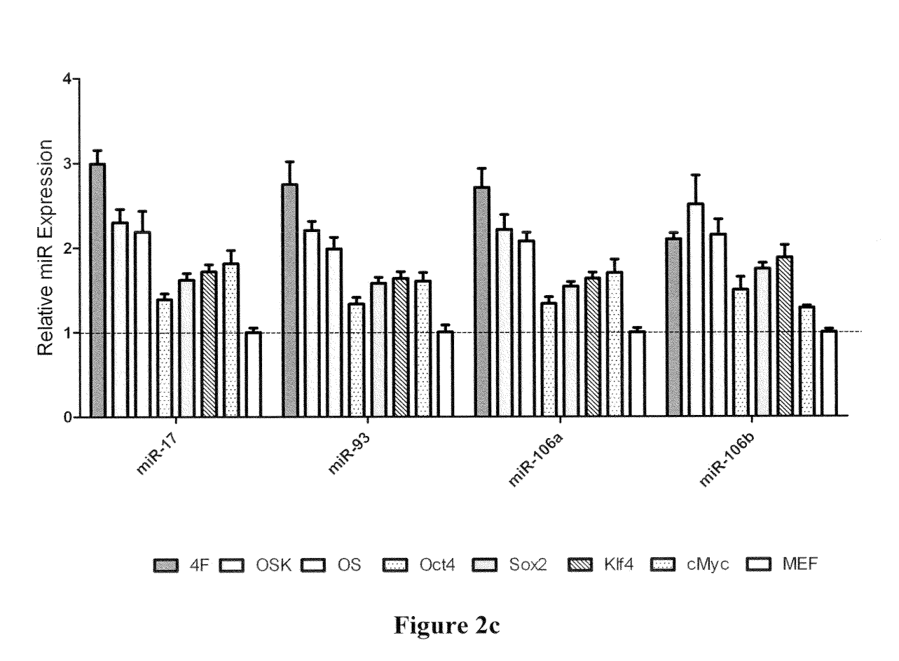

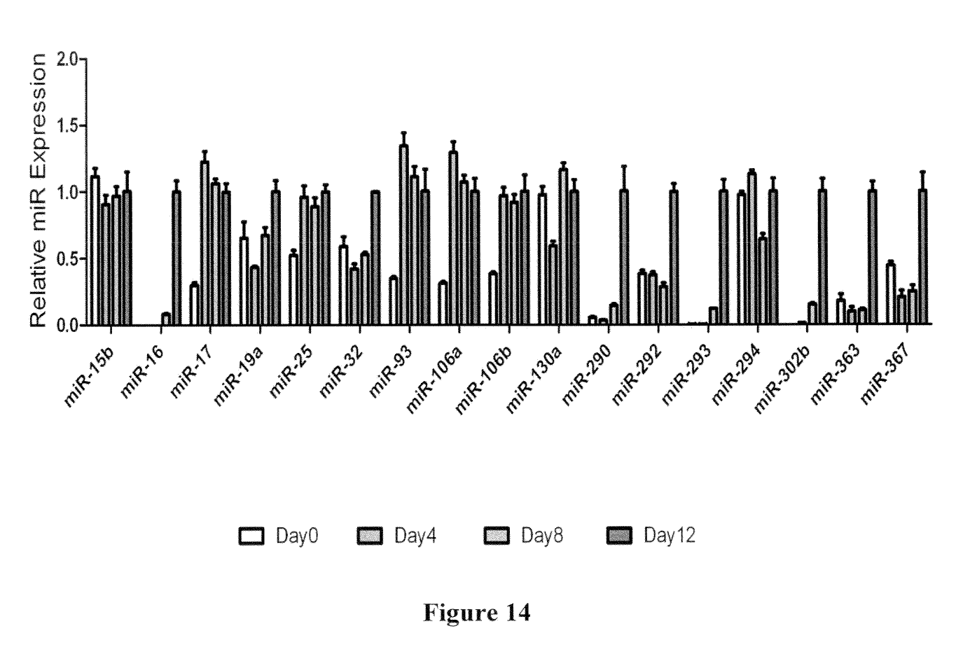

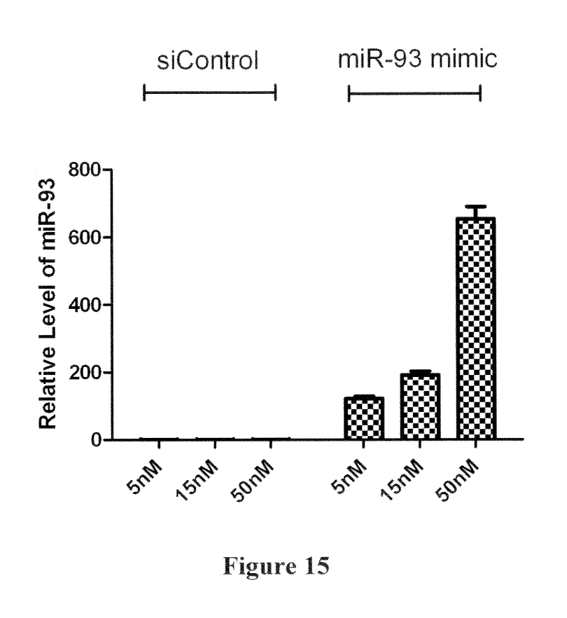

Two representative microRNAs from each cluster are evaluated using a miR qPCR based method to quantify the expression changes at different reprogramming stages, including day 0, day 4, day 8 and day 12—following transduction of the OSKM factors. Many ES-specific microRNAs, such as miR-290 cluster and miR-293 cluster, are not induced until day 8 (FIG. 14), at which stage GFP+ colonies are already detectable. Several other microRNA clusters, including miR-17˜92, 25˜106b, 106a˜363 and 302b˜367, are expressed to varying extents by day 4 post four factor transduction (FIG. 2a). Among these four microRNA clusters, the level of miR-302b˜367 in MEF is the lowest. Among the three clusters highly induced at reprogramming day 4, some shared very similar seed regions (FIG. 2b), suggesting that they function in reprogramming and can target similar sets of genes.

TABLE 2
List of microRNAs used for iPSC experiments

| | |
|---|---|
| mmu-miR-290 cluster | mmu-miR-290 |
| | mmu-miR-291a |
| | mmu-miR-292 |
| | mmu-miR-291b |
| mmu-miR-293 cluster | mmu-miR-293 |
| | mmu-miR-294 |
| | mmu-miR-295 |
| mmu-miR-302 cluster | mmu-miR-302b |
| | mmu-miR-302c |
| | mmu-miR-302a |
| | mmu-miR-302d |
| | mmu-miR-367 |
| mmu-miR-17-92 cluster | mmu-miR-17 |
| | mmu-miR-18a |
| | mmu-miR-19a |
| | mmu-miR-20a |
| | mmu-miR-19b |
| | mmu-miR-92a |
| mmu-miR-106a cluster | mmu-miR-106a |
| | mmu-miR-18b |
| | mmu-miR-20b |
| | mmu-miR-19b |
| | mmu-miR-92a |
| | mmu-miR-363 |
| mmu-miR-93 cluster | mmu-miR-106b |
| | mmu-miR-93 |
| | mmu-miR-25 |
| mmu-miR-15b cluster | mmu-miR-15b |
| | mmu-miR-16 |
| mmu-miR-130a | |
| mmu-miR-32 | |

Analysis is then performed to determine which of the four factors is responsible for induction of these microRNAs. By transducing MEFs with different combinations of the four factors at the same dose, total RNAs are harvested at day 4 post infection for miR qPCR analysis (FIG. 2c). This analysis confirms that cMyc alone can induce miR-17˜92, miR-25˜106b and miR-106a˜363 clusters expression. However, in all cases, a combination of all four reprogramming factors induced the most abundant expression of microRNA clusters, and that robust expression is correlated with the highest reprogramming efficiency (FIG. 2c).

These results identified that three microRNA clusters, including miR-17˜92, 25˜106b, 106a˜363 are induced during early stage of reprogramming, and further that the expression of these microRNAs is most highly induced by four factors together, although single factors can also induce their expression to a lesser extent.

### Example 5

### MicroRNAs Enhance IPSC Induction

MicroRNAs miR-93 and miR-106b are determined to enhance mouse iPSC induction. Since the four identified microRNA clusters contain several microRNAs with similar seed regions, the miR-106b˜25 cluster is further analyzed because this cluster includes 3 microRNAs (i.e., miR-25, miR-93 and miR-106b). MiR-93 and miR-106b have the identical seed region, and both are highly induced by the four reprogramming factors (FIG. 2a). It is provided that if these microRNAs are functioning in reprogrammed cells, an increased efficiency of iPSC induction is expected by introducing these microRNAs during the process.

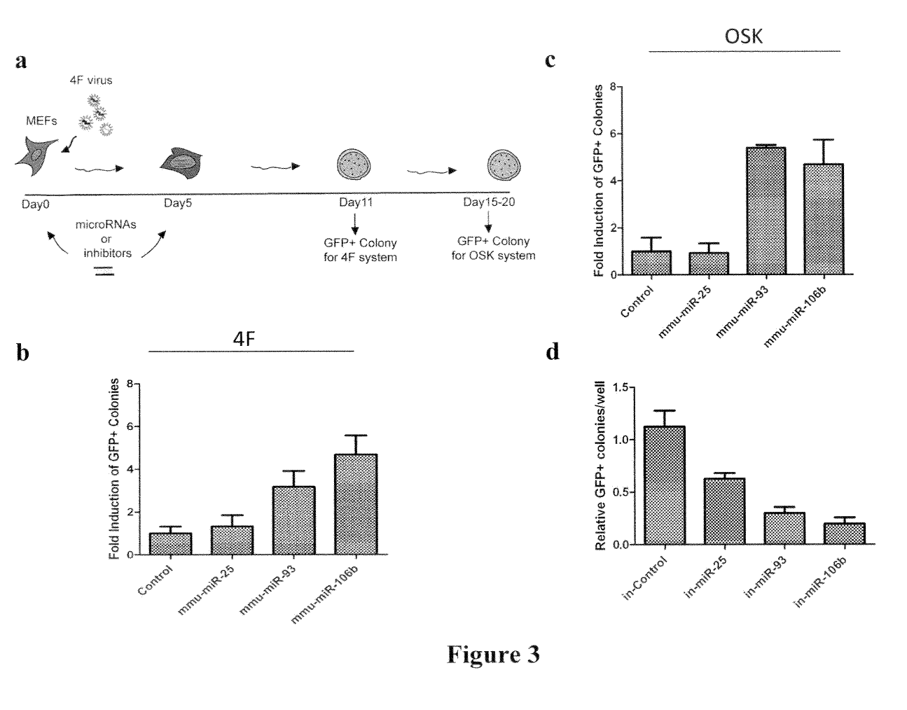

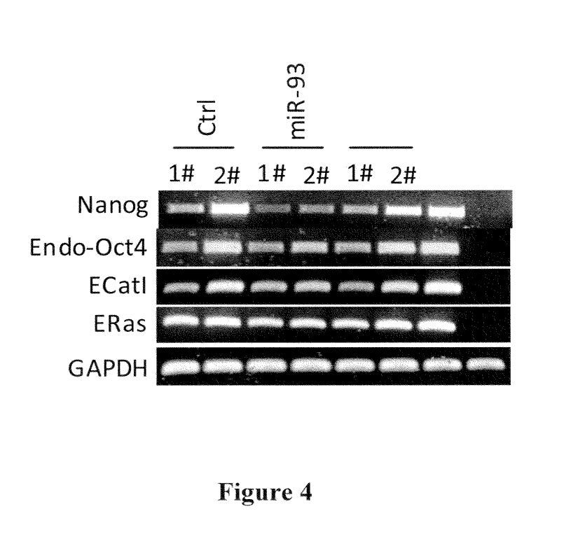

A strategy for directly transfecting microRNA mimics into MEFs is used for functional test of these induced microRNAs (FIG. 3a). MicroRNAs are introduced twice at day 0 and day 5 together with the four factor (or OSK) virus and a reporter MEF which has GFP expression under control of endogenous Oct4 promoter was used. For example, microRNA mimics are directly transfected into MEFs harboring Oct-4-GFP at days 0 and 5 with vectors expressing either all four factors (4F, OSKM) or only Oct4, Sox2, and Klf4 (OSK) and assayed reprogramming based on GFP expression. When these cells were successfully reprogrammed into iPSCs, they become GFP positive (+). GFP+ colonies are quantified around day 11 to evaluate the reprogramming efficiency (FIG. 3b; Table 3). Indeed, transfection of miR-93 and miR-106b mimics resulted in about 4˜6 fold increase of GFP+ colonies both in 4F and OSK transduction (FIG. 3c), confirming that these microRNAs which are induced during iPSC induction, facilitate MEF reprogramming.

TABLE 3
Number of GFP+ colonies with miRs for iPSC induction

| | | Experiment 1* | | | Experiment 2* | | |
|---|---|---|---|---|---|---|---|
| GFP+ | miRcontrol | 20 | 29 | 35 | 7 | 10 | 17 |
| colonies | miR-25 | 43 | 44 | N/A | 6 | 17 | 19 |
| | miR-93 | 175 | 70 | 42 | 84 | 35 | 26 |
| | miR-106b | 127 | 78 | 83 | 44 | 52 | 42 |

*4 × 10⁴ MEFs/well in 12-well plates (gelatin coated)

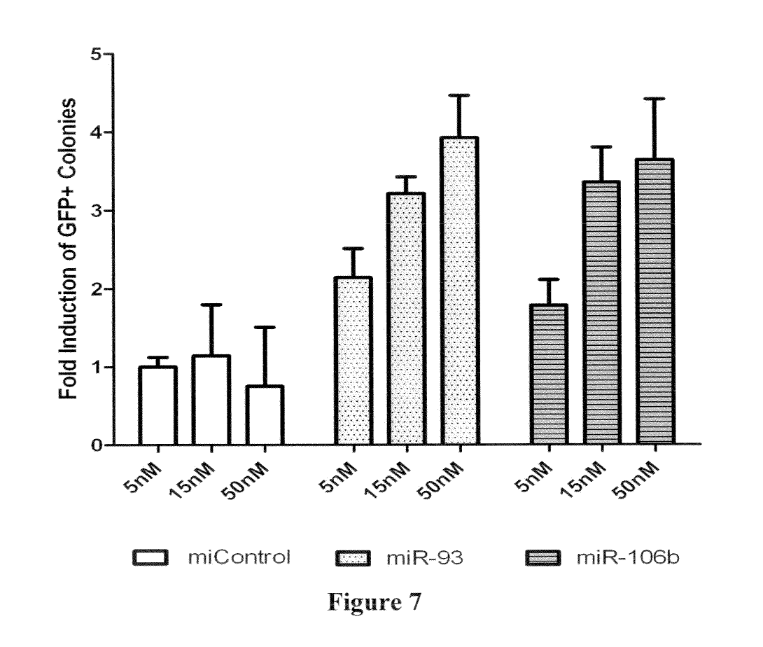

A dose dependent experiment shows that the enhanced reprogramming efficiency can be seen at as low as 5-15 nM range of miRs (FIG. 7). When the colonies are stained with alkaline phosphatase substrates, there appears to be no significant increase of AP+ colonies for miR mimic transfections, suggesting that miR-93 and miR-106b can facilitate the maturation process of iPSC colonies. This is also supported by the phenomenon observed using the OSK system, in which many GFP+ colonies appear at day 15 post OSK transduction in miR mimic transfected cells, while control wells did not exhibit any mature iPSC colonies at this stage.

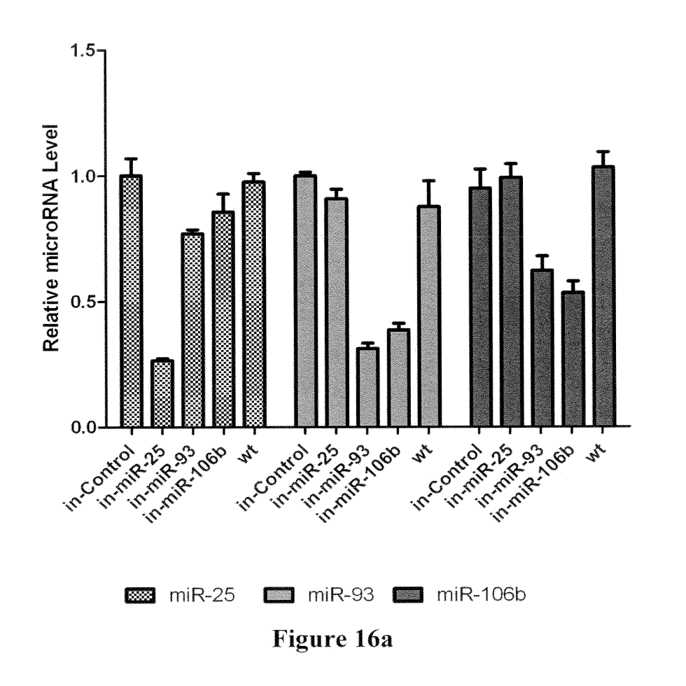

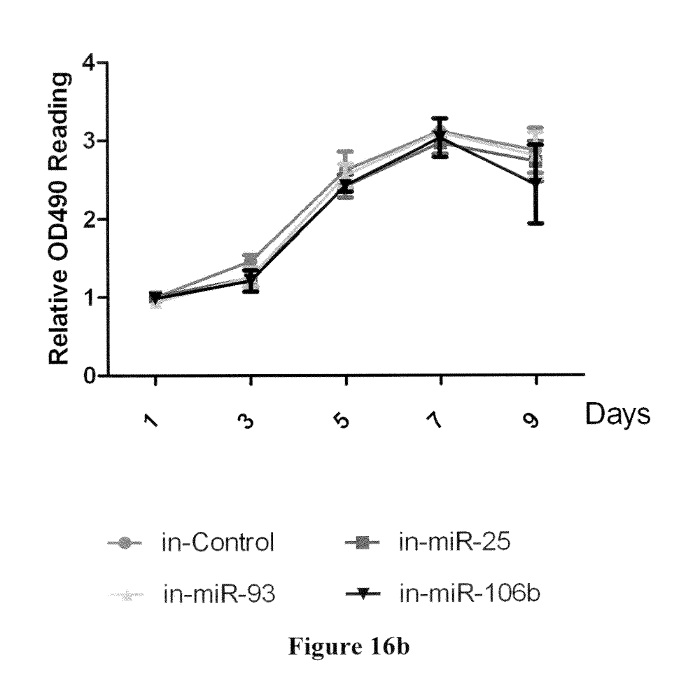

To confirm that these microRNAs are important in iPSC induction, miR inhibitors are also used to knock down targeted microRNAs during the process. All of the miR inhibitors tested can efficiently decrease target miR expression and their transfection does not affect proliferation (FIGS. 16a and 16b). Consistent with miR mimic experiments, miR-93 and miR-106b knock-down can promote a dramatic decrease of GFP+ colonies (FIG. 3d). Although the miR-25 mimic dose not enhance MEF iPSC induction, knocking down this microRNA decreases the reprogramming efficiency by about ˜40% (FIG. 3d), suggesting that miR-25 can also function during the reprogramming process. As a control, Let7a inhibitor did not have any effect on the reprogramming efficiency. These data strongly indicate that miR-93 and miR-106b promote reprogramming of MEFs to iPSCs. Reprogramming efficiency may be further enhanced by modulating these microRNAs during iPSC induction.

### Example 6

### MicroRNA-Derived Clones Are Fully Pluripotent

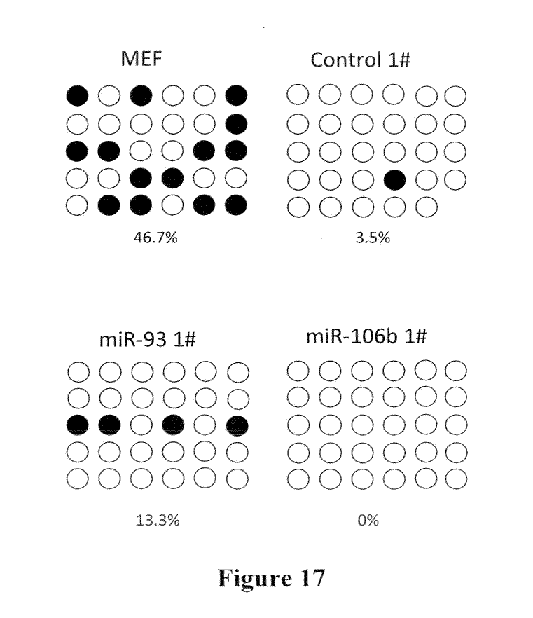

To examine whether induced cells reach a fully pluripotent state, several iPSC clones for each microRNA as well as miR controls are derived and analyzed for expression of pluripotency markers. All clones are GFP+ indicative of reactivated Oct4 expression. Immunostaining confirmed that Nanog and SSEA1 are also activated in all clones. RT-qPCR for other mES markers such as Eras, ECat I and endogeneous Oct4 show similar results. Whole genome mRNA expression profiling also indicates that derived clones exhibit a gene expression pattern more similar to mouse ES cells than MEFs. Promoter methylation of endogenous Nanog loci is analyzed, and all tested clones showed de-methylated promoters, as is observed in mouse ES cells (FIG. 17).

To investigate whether derived clones exhibit the full differentiation capacity of mES cells, embryoid body (EB) formation is evaluated. All derived clones show efficient EB formation, and EBs show positive staining for lineage markers such as such as β-tubulin III (ectoderm), AFP (endoderm) and a-actinin (mesoderm). Beating EBs were also derived from these cells, indicating that functional cardiomyocytes can be derived from these miR-iPSC clones. When these miR-iPSCs are injected into athymus nude mice, teratomas are readily derived in 3-4 weeks. Finally, as a more stringent test, miR-derived iPSC clones are injected into albino/black B6 blastocysts and generated chimera mice. Furthermore, these cells could contribute to the genital ridge of derived E13.5 embryos. These results indicate that the enhancing effects of miR-93 and miR-106b on reprogramming do not alter differentiation capacity of induced pluripotent cells and that those derived clones can differentiate into all three germ lines.

### Example 7

### MiR-93 and MiR-106b Target Tgfbr2 and P21 in Mice

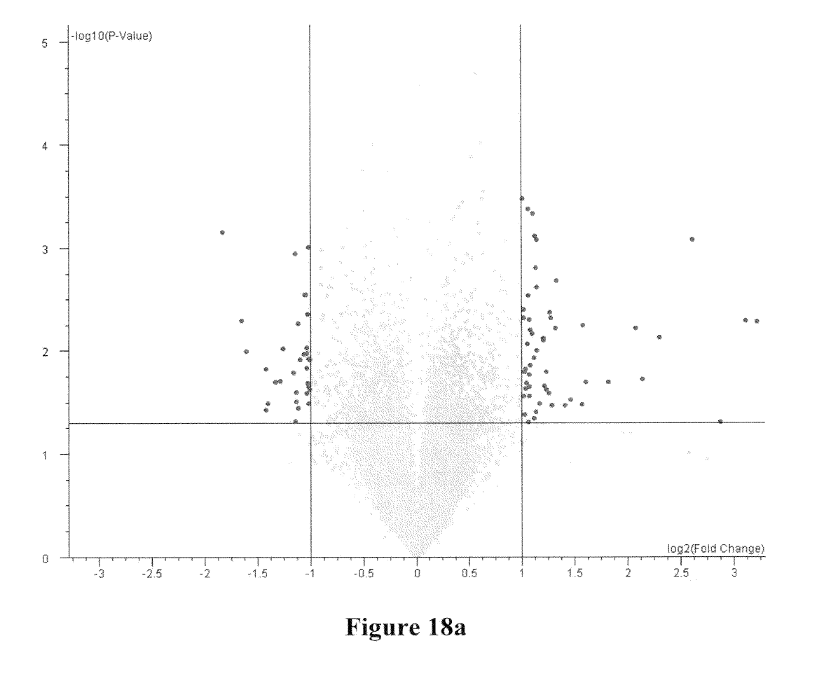

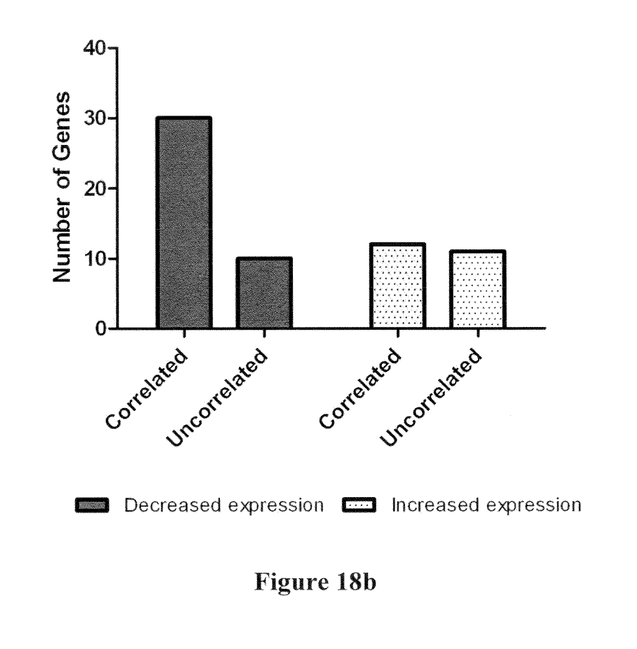

To further understand the mechanism underlying miR-93 and miR-106b enhancement of reprogramming efficiency, the cellular targets of these microRNAs are investigated. MiR-93 is first chosen for analysis since it shares the same seed region as miR-106b. MiR-93 mimics are transfected into MEFs, and total RNAs are harvested at day 2 for mRNA expression profile analysis. That analysis identifies potential functional targets of miR-93 as compared with published expression profiles of MEFs and iPSCs. Genes significantly decreased upon miR-93 transfection show a threefold enrichment of genes which are lowly expressed in iPSCs (FIG. 18a), while genes which are increased upon miR-93 transfection do not show such enrichment. In addition, pathway ontology analysis is performed for the expression profile of miR-93 transfected MEFs. Interestingly, two important pathways for iPSC induction are regulated by miR-93: TGF-β signaling and G1/S transition pathways.

For TGF-β signaling, Tgfbr2 is among one of the most significantly decreased genes upon miR-93 transfection. Tgfbr2 is a constitutively active receptor kinase that plays a critical role in TGF-β signaling, and recent small molecule screens indicate that inhibitors of its heterodimeric partner Tgfbr1 enhance iPSC induction. MicroRNA target site prediction suggests that there are two conserved targeting site for miR-93 and its family microRNAs in its 3′UTR. Therefore miR-93 is chosen as the first candidate target for further investigation.

Regarding the G1/S transition, p21 is chosen as the potential target because recent results in human solid tumor samples (breast, colon, kidney, gastric, and lung) and gastric cancer cell lines indicate that the miR-106b˜25 cluster can target cell cycle regulators, such as the CDK inhibitors p21 and p57 and that human and mouse p21 share a conserved miR-93/106b target site in the 3′UTR.

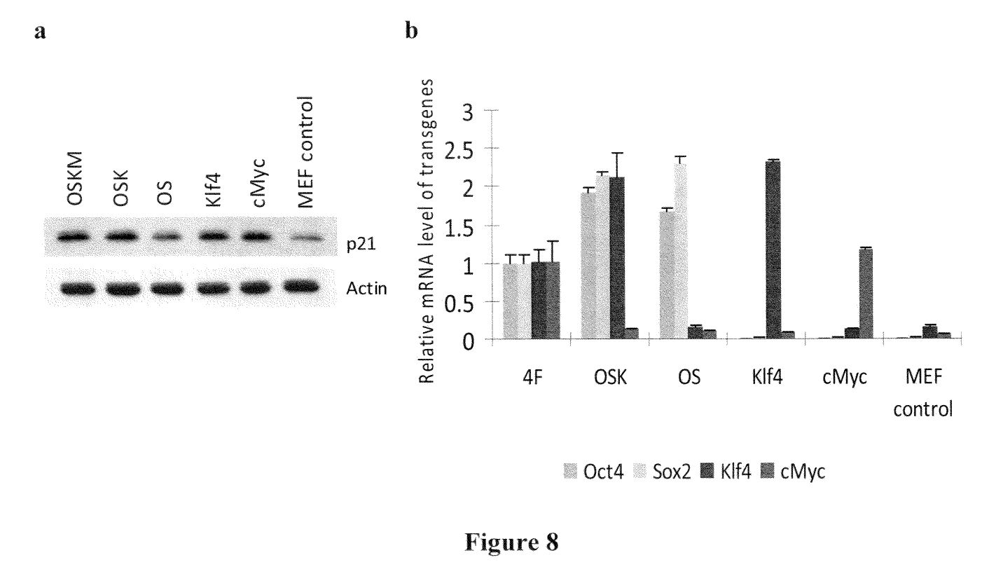

Furthermore, mouse ES cell-specific microRNA clusters, including miR-290 and miR-293 clusters, have also been proposed to target several G1-S transition negative regulators including p21. Additionally, miR-290 and 293 cluster microRNAs share very similar seed regions with miR-93 and miR-106b. Therefore, p21 is also analyzed as a candidate target. Further, p21 is greatly induced by the four factors OSKM during early stage of iPSC induction (FIG. 8a). Detailed analysis reveals that induction of p21 is mainly due to overexpression of Klf4 and cMyc, as combinations of Oct4 and Sox2 do not show a significant change of p21 level (FIG. 8a).

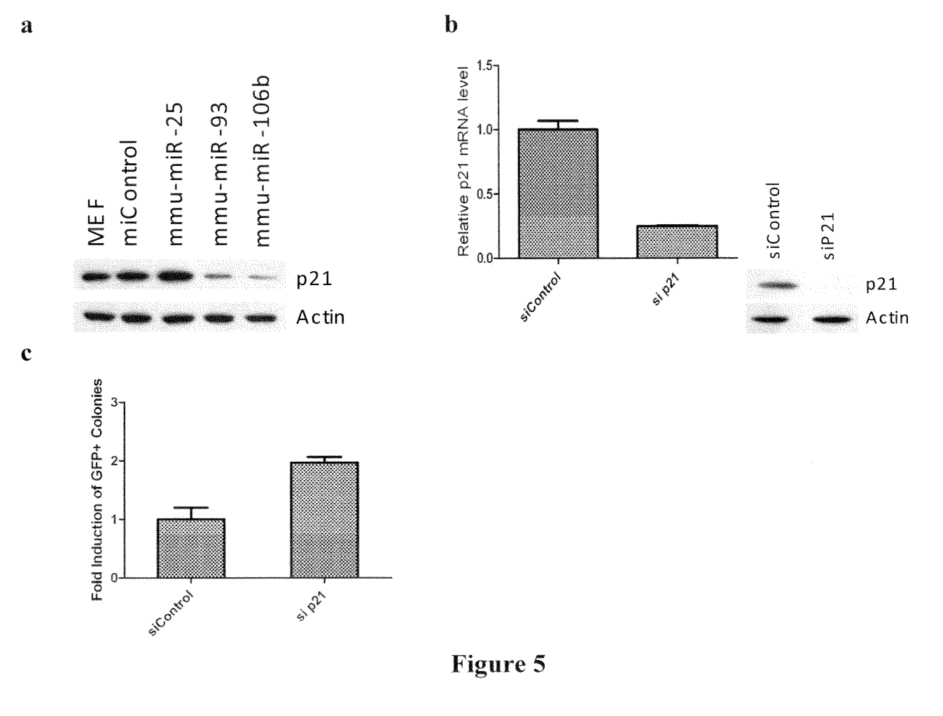

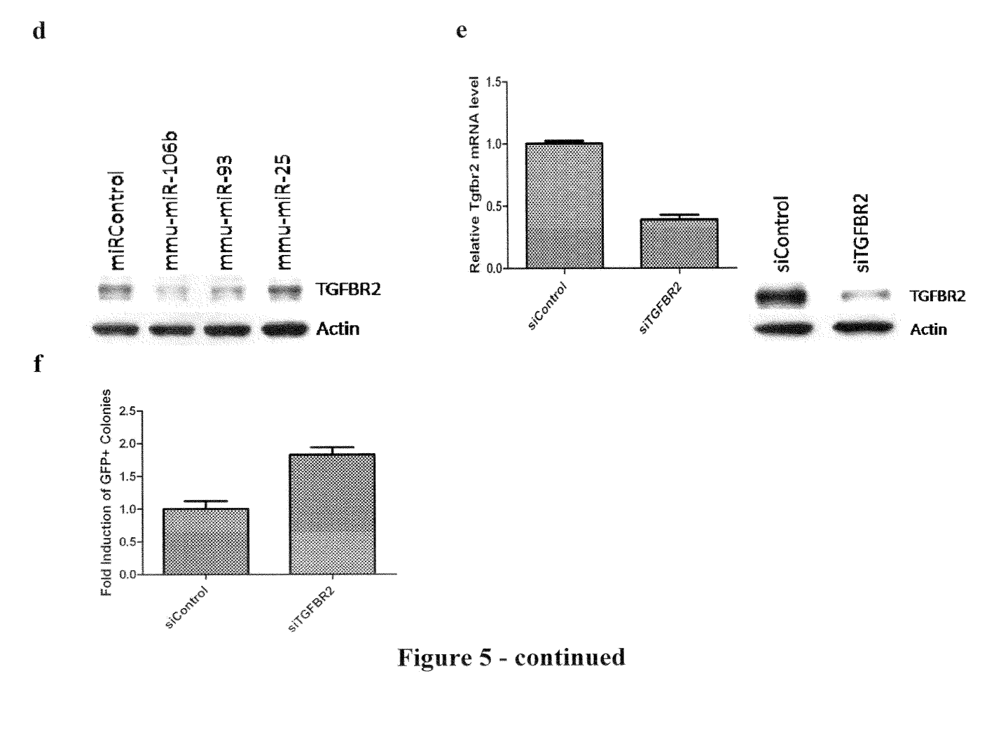

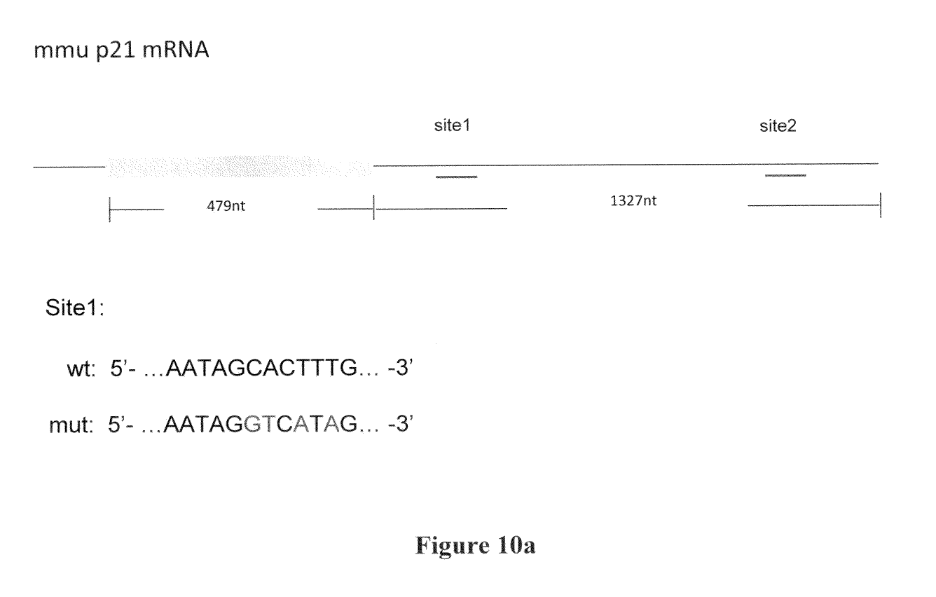

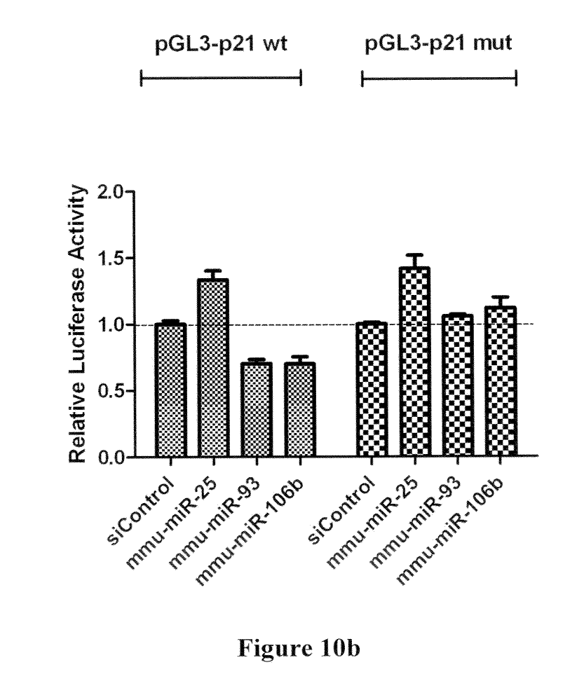

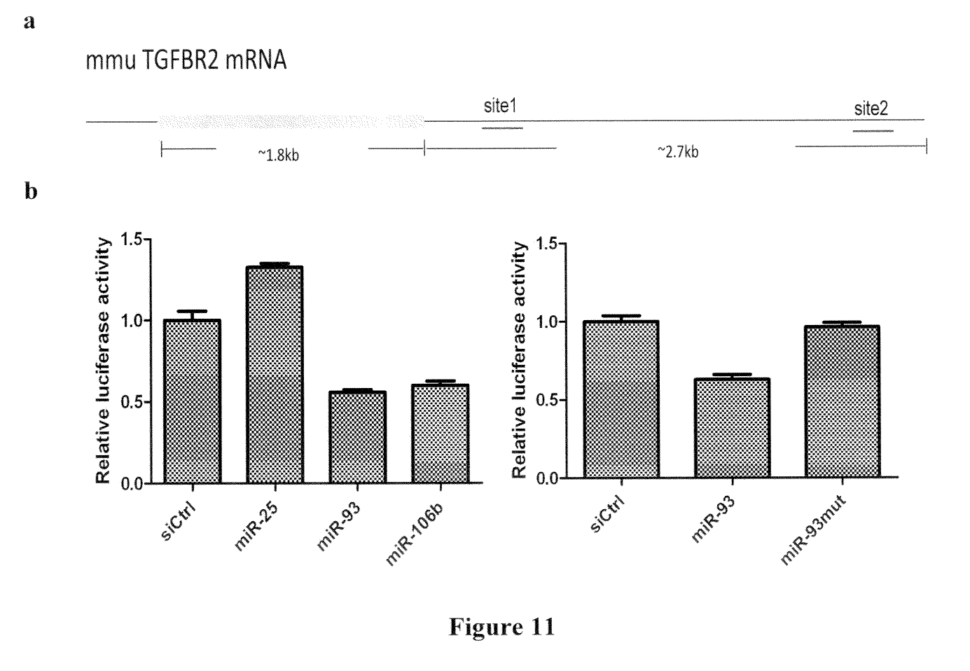

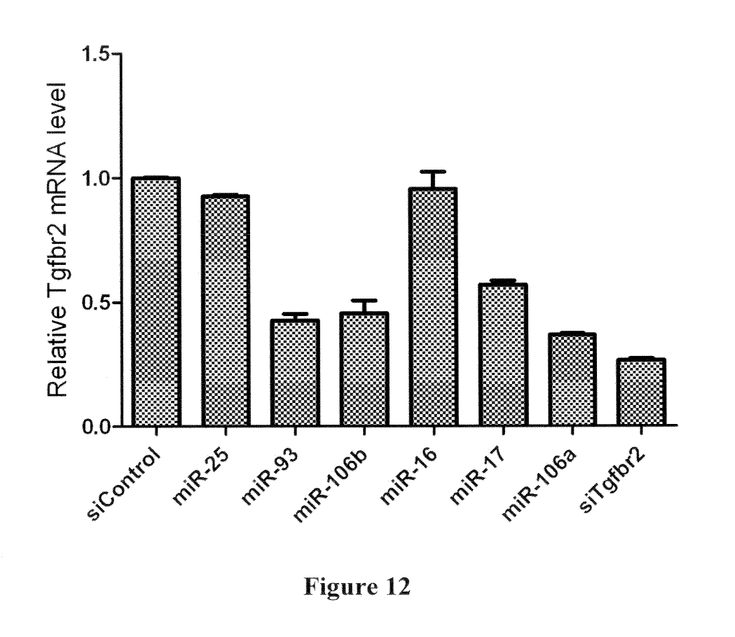

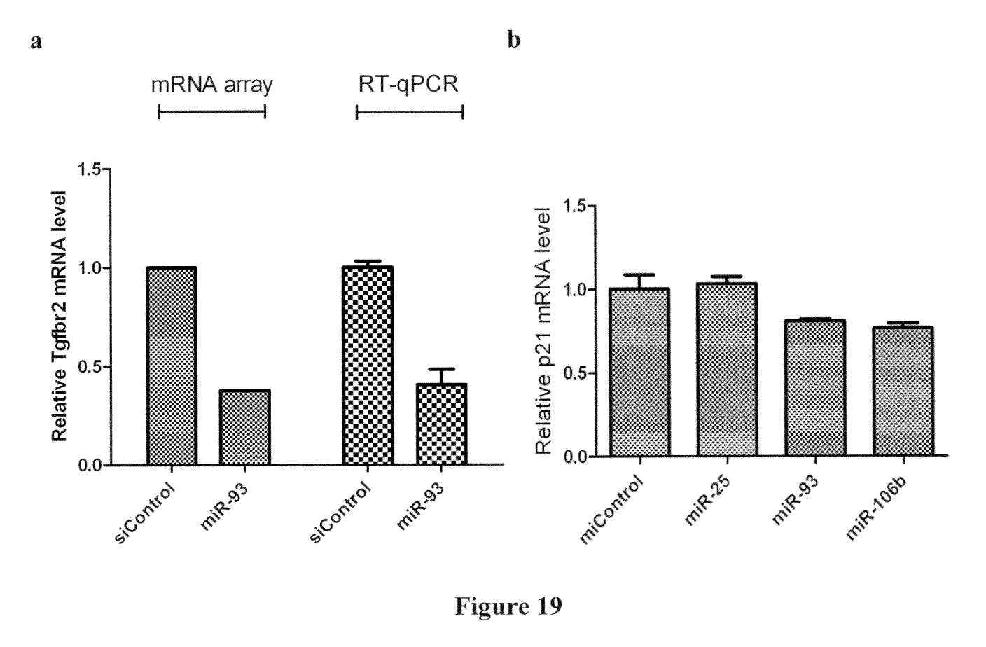

To verify whether mouse Tgfbr2 and p21 are targeted by miR-93 and miR-106b, miR mimics are transfected into MEFs and total cell lysates are analyzed after 48 hrs by western blotting. Indeed, miR-93 and miR-106b efficiently decrease protein level of both Tgfbr2 and p21 (FIGS. 5a and 5d) and also have a ˜25-30% reduction of p21 mRNA level and a ˜60-70% reduction of Tgfbr2 mRNA level (FIG. 19). To further investigate whether p21 is the direct target of miR-93 and miR-106b, a luciferase assay is performed where a luciferase reporter with p21 3′ UTR sequence inserted down stream of the firefly luciferase coding sequence. The luciferase assay reveals that a consistent ˜40% repression of luciferase activity may be achieved by transfecting miR mimics in Hela cells. It is also determined that the repression of microRNA mimics may be disrupted completely when mutations are introduced into seed region of the conserved p21 3′UTR target site (FIG. 10). For Tgfbr2, the luciferase assay also shows ˜50% decrease of GL activity while miR-93 mutants do not have such effect (FIG. 11).

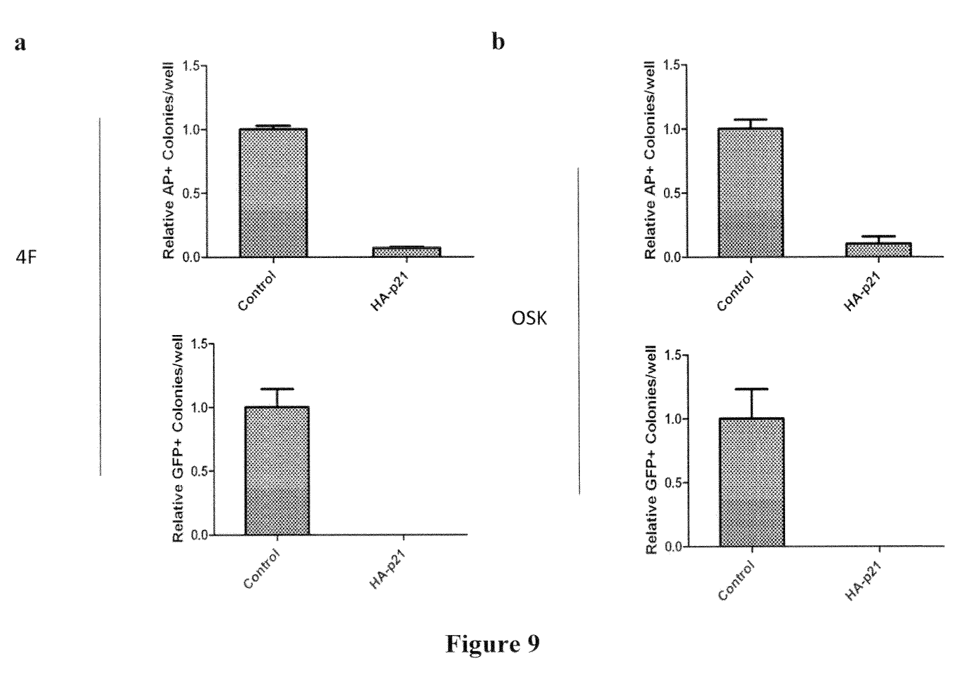

Cell cycle arrest promoted by p21 may inhibit epigenetic modifications required for reprogramming, since those modifications occur more readily in proliferating cells. To determine whether p21 expression compromises iPSC induction, HA-tagged p21 cDNA is cloned into the pMX retroviral backbone and overexpressed in MEF cells. When HA-p21 virus is introduced into MEFs together with the four OSKM factors, an almost complete inhibition of iPSC induction is observed, based on both alkaline phosphatase staining and Oct4-GFP-positive colony formation (FIG. 9a). Similar results are obtained when the three OSK factors are used for reprogramming (FIG. 9b).

Since the analysis indicates that miR-93 and miR-106b efficiently repress both Tgfbr2 and p21 expression, Tgfbr2 and p21 are further examined whether their activity can antagonize reprogramming. Tgfbr2 or p21 siRNAs are transfected into MEFs using the same experimental time line employed with microRNA mimics. Western blotting and RT-qPCR confirm that both protein and mRNA levels, respectively, are efficiently knocked down by siRNAs without virus transduction (FIGS. 5b and 5e). MEF reprogramming is then initiated by OSKM transduction, and Oct4-GFP+ colonies are quantified at day 11 post-transduction. A ˜2-fold induction in colony number for each gene is observed (FIGS. 5c and 50. All together, our data identify that Tgfbr2 and p21 are the direct target of miR-93 and miR-106b and down regulation of these genes can enhance the reprogramming process.

### Example 8

### Pluripotency of IPSC Clones Derived From MiR-93 and MiR-106b Transfection

Although miR-93 and 106b have been confirmed about their ability to enhance mouse iPSC induction, a remaining question is whether the induced cells reach the full pluripotent state or not. To answer this question, several iPSC clones for each microRNA as well as miR control are derived to analyze pluripotency markers and differentiation capacity. These derived clones are all GFP+ which indicates a reactivation of Oct4 locus. Immunostaining also confirmed that Nanog and SSEA1 are also activated in these cells. RT-qPCR for other mES markers shows similar results. Whole genome mRNA expression profile again indicates that these derived clones have very similar gene expression pattern with mES but not MEFs. Promoter methylation of endogenous Oct4 and Nanog locus are also analyzed and all the tested clones were observed to have de-methylated promoters.

To investigate whether those derived clones have the full differentiation capacity of mES cells, embryonic body formation is first used as an initial test. Derived clones all give efficient formation of EBs and those EBs are determined to be positive for the lineage markers staining. Beating EBs can also be derivable from these cells.

Finally, as a more stringent test, these derived clones are injected to check whether they contribute to the chimera mice or not. Indeed, chimeras are derivable from all the clones tested. These results prove that miR-93 and miR-106b′ s enhancing effects on reprogramming does not change the capacity of induced cells, and also that derived clones having reached an ES-like state can differentiate to all the three lineages.

### Example 9

### Up-Regulation of Other MicroRNAs Also Enhances IPSC Induction

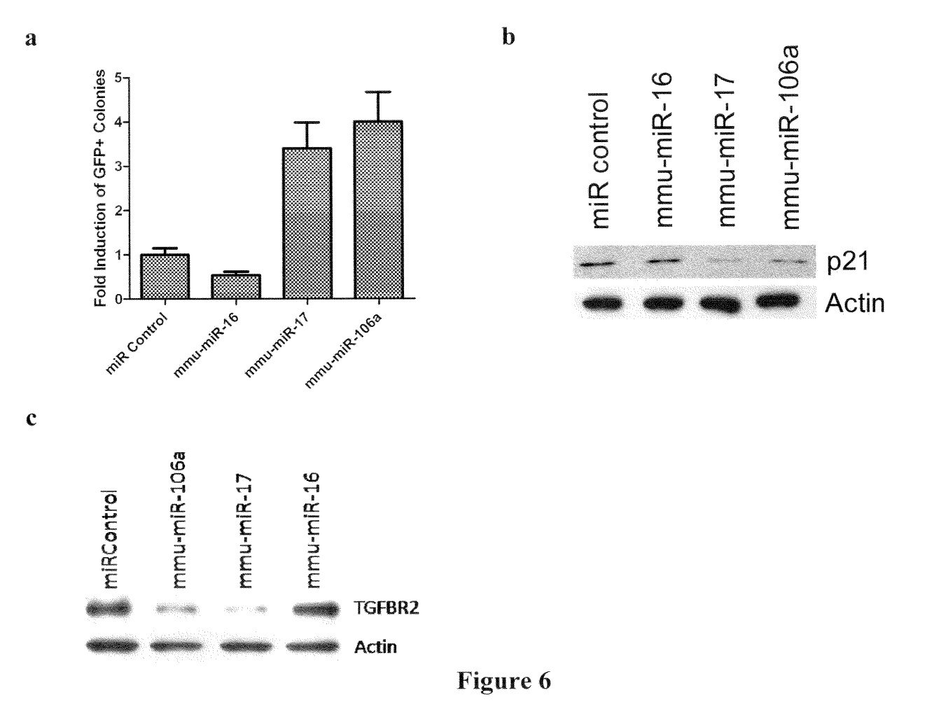

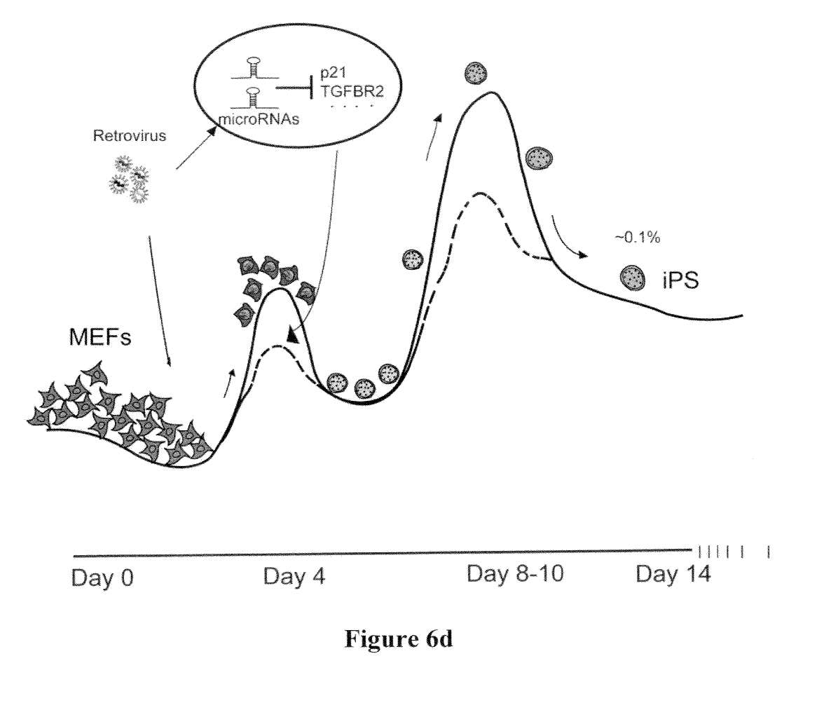

As discussed herein, three clusters of microRNAs are identified to be induced by four factors during iPSC induction and several microRNAs within these clusters have been determined to have the same seed regions indicating they target to similar mRNAs (FIG. 2). To investigate whether other microRNAs which share the same seed region with miR-93 and miR-106b can similarly enhance iPSC induction, microRNA mimics of miR-17 and miR-106a are tested using an experimental procedure similar to that described above for miR-93 mimic treatment and iPSC induction. Indeed, these microRNAs enhance reprogramming in a manner similar to that seen with the miR-106b˜25 clusters (FIG. 6a), and transfection of these miRs all results in decreased Tgfbr2 and p21 protein levels (FIGS. 6b and 6c).

Together, these results suggest that inductions of miR-17˜92, miR-106b˜25 and miR-106a˜363 clusters are important for proper reprogramming and that up-regulation of these microRNAs lower reprogramming barriers to the iPSC generation process (FIG. 6d). Therefore, the level of these microRNAs in the cells may be manipulated to improve reprogramming efficiency.

### Example 10

### Mechanisms of IPSC Reprogramming

Derived clones are shown to activate endogenous Oct4-GFP expression. Colonies are picked starting at day 12 post-OSKM transduction with microRNA mimics and maintained on irradiated MEF feeder plates. Green fluorescence can be observed as GFP signal from the endogenous pou5f1 locus. Clones can be shown using alkaline phosphatase staining and immunostaining of ES-specific markers based on Nanog and SSEA1 staining. Hoechst 33342 can be used for nuclear staining. Cells from all three germ layers can be obtained in embryoid body (EB) assays using derived iPSC clones. iPS cells are cultured for EB formation at ˜4000 cells/20 μl drop for 3 days, and EBs are then reseeded onto gelatin coated plates for further culture until day 12-14, when beating cardiomyocytes are observed. Cells can be immunostained with different lineage markers, including β-tubulin III for an ectoderm marker; AFP for an endoderm marker; and a-Actinin for a mesoderm marker. Teratomas can form from injected iPS cells, where 1.5 million cells are injected into each mouse, and tumors are harvested 3˜4 weeks after injection for paraffin embedding and H&E staining. Derived clones can also be used to generate chimeric mice. iPS cells are injected into blastocysts from albino or black C57B6 mice (NCI) and the contribution of iPSCs can be seen with agouti or black coat color.

Reprogrammed cells at day 12 can be stained with alkaline phosphatase substrates.

The present invention provides that miR mimics transfection do not cause significant increase of AP+ colonies, however, knock-down of miR-93 and 106b results in significant loss of AP+ colonies as well as GFP+ colonies. MicroRNA mimics do not affect overall AP+ colony formation while inhibitors do.

Since the discovery that MEFs can be reprogramming to iPS cells, much efforts have been directed toward understanding the fundamental mechanism for this magnificent process. The results described herein have identified for the first time that post-transcriptional gene regulation is directly involved during reprogramming and that interference with the RNAi machinery can significantly alter reprogramming efficiency. Additionally, as shown in the previous examples three clusters of microRNAs are significantly up-regulated by the four factors used to induce iPS cells, and microRNAs in these clusters likely target at least two important pathways: TGF-β signaling and cell cycle control.

While this work has been pursued, several recent reports have also identified that the p53 pathway, which includes several downstream tumor suppressors such as p21, is one of the major barriers during iPSC induction. Much evidence indicates that ectopic expression of the four factors (OSKM) readily up-regulates p53 and initiates serial reactions of cellular defense programs such as cell cycle arrest, apoptosis, or DNA damage responses. These defense responses likely underlie low reprogramming efficiency, which is believed around ˜0.1%. However, these data do not explain how successfully reprogrammed cells manage to overcome those cellular barriers in order to become iPS cells. The examples described herein show that these cells may overcome those barriers, at least in part if not all, by inducing the expression of microRNAs that target pathways that antagonize successful reprogramming. By modulating microRNAs levels in primary fibroblasts, a significant increase of the reprogramming efficiency may be achieved.

TGF-β signaling is an important pathway that functions in processes as diverse as gastrulation, organ-specific morphogenesis and tissue homeostasis. The current model of canonical TGF-β transduction indicates that TGF-β ligand binds the TGF-β receptor II (Tgfbr2), which then heterodimerizes with Tgfbr1 to transduce signals through receptor-associated Smads. TGF-β signaling reportedly functions in both human and mouse ES cell self-renewal, and FGF2, a widely used growth factor for ES cell culture, induces TGF-β ligand expression and suppresses BMP-like activities. Blocking TGF-β receptor I family kinases by chemical inhibitors compromises ES cell self-renewal. These findings are particularly significant for iPSC induction, because those inhibitors seem to have completely different roles during reprogramming. Recent chemical screening has shown that small molecules inhibitors of the TGF-β receptor I (Tgfbr1) actually enhance iPSC induction and can replace the requirement for Sox2 by inducing Nanog expression. Moreover, treating reprogramming cells with TGF-β ligands has a negative effect on iPSC induction. Therefore, although TGF-β signaling is important for ES cell self-renewal, it is a barrier for reprogramming. The present invention provides that, in addition to Tgfbr1, activity of the constitutively active kinase Tgfbr2 also antagonizes reprogramming. The present invention also provides that miR-93 and its family members directly target Tgfbr2 to modulate it's signaling and reprogramming.

P21, which is a small protein with only 165 amino acids, has long been discovered as a tumor suppressor during cancer development by causing p53-dependent G1 growth arrest and promoting differentiation and cellular senescence. The present invention provides that p21 expression is up-regulated when four factors (OSKM) are introduced into MEF cells and this up-regulation antagonizes the reprogramming process (FIG. 8), since overexpression of p21 almost completely block iPSC induction (FIG. 9). The induction of p21 in the reprogramming cells can be dependent or independent of p53 as the Klf4 reprogramming factor binds to the p21 promoter and increase p21 transcription.

This raises an interesting question about the function of the four reprogramming factors, since the same transcription factor can promote iPSC induction and antagonize iPSC induction. In fact, current evidences cannot rule out the possibility that a certain level of p21 induction can be beneficial to the reprogramming process. Besides its well-known role in p53 dependent cell cycle arrest, p21 has also been reported to have some oncogenic activities. For example, p21 also has an oncogenic activity by protecting cells from apoptosis, a function unrelated to its usual function in the cell cycle control.

A potential benefit for p21 in reprogramming may depend on its ability to regulate gene expression through protein-protein interactions. For example, p21 can directly bind to several proteins which are involved in apoptosis, such as caspase 8, caspase 10 and procaspase 3. For another example, p21 is also a suppressor of Myc's pro-apoptotic activity by association with the Myc N-terminus to block Myc-Max heterodimerization. Indeed, when Myc itself is overexpressed in MEFs, a significant increase of cell death can be noticed in the cell culture, while in four factor transduced cells, cell death is minimal compared with myc-only samples. Therefore, induction of p21 may not only serve as a barrier to the reprogramming process but also may maintain certain levels of p21 necessary to reduce cell apoptosis and thus increase the reprogramming efficiency.

The data provided herein may also can be seen as a partial evidence to support this hypothesis, as transfection of miR-93 and miR-106b have greater enhancing effects on reprogramming than p21 siRNA transfection, in which miR-93 and 106b did not suppress p21 expression as much as p21 siRNA. However, it is also possible that this effect is due to targeting of multiple proteins including Tgfbr2 and p21 by these microRNAs.

Since microRNAs usually target to multiple cellular proteins, the enhancing effects of miR-93 and miR-106b provide an opportunity to find additional genes which are involved in reprogramming in order to better understand the process. Indeed, besides p21, several other genes which are reported to be negative regulators of G1-S transition, also have miR-93 and miR-106b target sites in the 3′UTR regions of the mRNA transcripts, such as Rb1, Rb11, Rb12 and Lats2. Another interesting reported target of miR-93 and miR-106b is transcription factor E2F1, which is frequently found to be deregulated and hyperactivated in many human tumor samples. One profound function of E2F1 is to activate the expression of CDKN2A locus, which encodes ARF and INK4a. Ink4a/Arf locus can also inhibit reprogramming efficiency. Thus, the present invention provides that transfection of miR-93 and miR-106b can also target to E2F1 and reduce the potential to activate CDKN2A locus and thus reduce the barriers of reprogramming.

Finally, miR-17˜92, miR-106b˜25 and miR-106a˜363 clusters are quite conserved between mouse and human. Therefore, the present invention provides that the enhancing effects of miR-93 and miR-106b may also apply to human reprogramming.

### Example 11

### MicroRNA Modulate IPS Cell Reprogramming

Mouse Embryonic Fibroblast (MEF) derivation: Oct4-EGFP MEFs are derived from the mouse strain B6; 129S4-Pou5f1^{tm2(EGFP)Jae}/J (Jackson Laboratory; stock #008214) using the protocol provided on the WiCell Research Institute website (www.wicell.org/). Oct4-EGFP MEFs are maintained on 0.1% gelatin-coated plates in MEF complete medium (DMEM with 10% FBS, nonessential amino acids, L-glutamine, but without sodium pyruvate).

Reprogramming using retrovirus: 4×10⁴ Oct4-EGFP MEFs are transduced with pMX retroviruses to misexpress Oct4, Sox2, Klf4, and c-Myc (Addgene). Two days later, transduced Oct4-EGFP MEFs are fed with ES medium (DMEM with 15% ES-screened FBS, nonessential amino acids, L-glutamine, monothioglycerol, and 1000 U/ml LIF) and the media are changed every other day. Reprogrammed stem cells (defined as EGFP+iPSC colonies) are scored by fluorescence microscopy ˜two weeks post transduction, unless otherwise stated. To derive iPSCs, EGFP+ colonies are manually picked under a stereo microscope (Leica).

MicroRNA inhibitor or siRNA transfection: Inhibitors of let-7a, miR-21, and miR-29a microRNAs are purchased from Dharmacon. 4×10⁴ Oct4-EGFP MEFs are transfected with Lipofectamine and inhibitors according to manufacturer's instruction (Invitrogen). Three to five hours later, the medium is discarded and replaced with MEF complete medium; for reprogramming, retrovirus encoding reprogramming factors (Oct4, Sox2, Klf4, and c-Myc) is added and the medium was changed to complete medium the next day. Inhibitors or siRNAs are introduced again at day 5 after transfection/transduction, unless otherwise stated.

For Northern analysis, 1×10⁵ Oct4-EGFP MEFs are transfected and harvested 5 days later. Total RNA is isolated by TRIZOL (Invitrogen) and ˜9 microgram of total RNA is resolved on a 14% denaturing polyacrylamide gel (National Diagnostics). RNAs are transferred onto Hybond-XL membranes (GE healthcare), and microRNAs are detected by isotopically-labeled specific DNA probes. Signal intensity is visualized by phospho-imager and analyzed using Multi Gauge V3.0 (FUJIFILM). MicroRNA signal intensity is normalized to that of U6 snRNA. Experiments are performed in triplicate.

For Western analysis, 1×10⁵ Oct4-EGFP MEFs are transfected and harvested 5 days later. Total proteins are prepared in M-PER buffer (Pierce), and equal amounts of total protein are separated on 10% SDS-PAGE gels. Proteins are transferred to PVDF membranes and bands are detected using the following antibodies: GAPDH (Santa Cruz; Cat# sc-20357), p53 (Santa Cruz; Cat# sc-55476), PI3 kinase p85 (Cell Signaling; Cat# 4257); Cdc42 (Santa Cruz; Cat# sc-8401); p-ERK1/2 (Cell Signaling; Cat#9101); ERK1/2 (Cell Signaling; Cat#9102); p-GSK313 (Cell Signaling; Cat#9323); GSK3β (Cell Signaling; Cat#9315); β Actin (Thermo Scientific; Cat#MS-1295). Signal intensity is quantified by Multi Gauge V3.0 (FUJIFILM) and normalized to GAPDH or β actin. Experiments are repeated three to five times.

In vitro differentiation and teratoma formation assay: For in vitro differentiation, iPSCs are dissociated by trypsin/EDTA and resuspended in embryoid body (EB) medium (DMEM with 15% FBS, nonessential amino acid, L-glutamine) to a final concentration of 5×10⁴ cells/ml. To induce EB formation, 1000 iPS cells in 20 microliters are cultured in hanging drops on inverted Petri dish lids. Three to five days later, EBs are collected and transferred onto 0.1% gelatin-coated 6-well plates at ˜10 EBs per well. Two weeks after formation of EBs, beating cardiomyocytes (mesoderm) are identified by microscopy, and cells derived from endoderm and ectoderm were identified by α-fetoprotein (R&D; Cat#MAB1368) and neuron specific β111-tubulin (abcam; Cat# ab7751) antibodies, respectively.

For teratoma assays, 1.5×10⁶ iSPCs are trypsinized and resuspended in 150 microliters and then injected subcutaneously into the dorsal hind limbs of athymic nude mice anesthetized with avertin. Three weeks later, mice are sacrificed to collect teratomas. Tumor masses are fixed, dissected and analyzed in the Cell Imaging-Histology core facility at the Sanford-Burnham Institute.

Chimera analysis: iPSC media is changed two hours before harvest. Trypsinized iPSCs are cultured on 0.1% gelatin-coated plates for 30 min to remove feeder cells. IPSCs are injected into E3.5 C57BL/6-cBrd/cBrd blastocysts and then transferred into pseudopregnant recipient females. After birth, the contribution of iPSCs is evaluated by pup coat color: black is from iPSCs.

Immunofluorescence and Alkaline Phosphatase (AP) staining: iPSCs are seeded and cultured on 0.1% gelatin-coated 6-well plates. Four days later, cells are fixed in 4% paraformaldehyde (Electron Microscopy Sciences; Cat# 15710-S). For immunofluorescence staining, fixed cells are permeablized with 0.1% Triton X-100 in PBS and blocked in 5% BSA/PBS. Antibodies against SSEA-1 (R&D; Cat# MAB2155) and Nanog (R&D; Cat# AF2729) serve as ES markers. Nuclei are visualized by Hoechst 33342 staining (Invitrogen). For AP staining, fixed cells are treated with alkaline phosphatase substrate following the manufacturer's instruction (Vector Laboratories; Cat# SK-5100).

### Example 12

### Inhibition of MiR-21 or MiR-29a Enhances Reprogramming Efficiency

Mouse Embryonic Fibroblast (MEF) derivation: Oct4-EGFP MEFs are derived from the mouse strain B6; 129S4-Pou5f1^{tm2(EGFP)Jae}/J (Jackson Laboratory; stock #008214) using the protocol provided on the WiCell Research Institute website (www.wicell.org/). Oct4-EGFP MEFs are maintained on 0.1% gelatin-coated plates in MEF complete medium (DMEM with 10% FBS, nonessential amino acids, L-glutamine, but without sodium pyruvate).

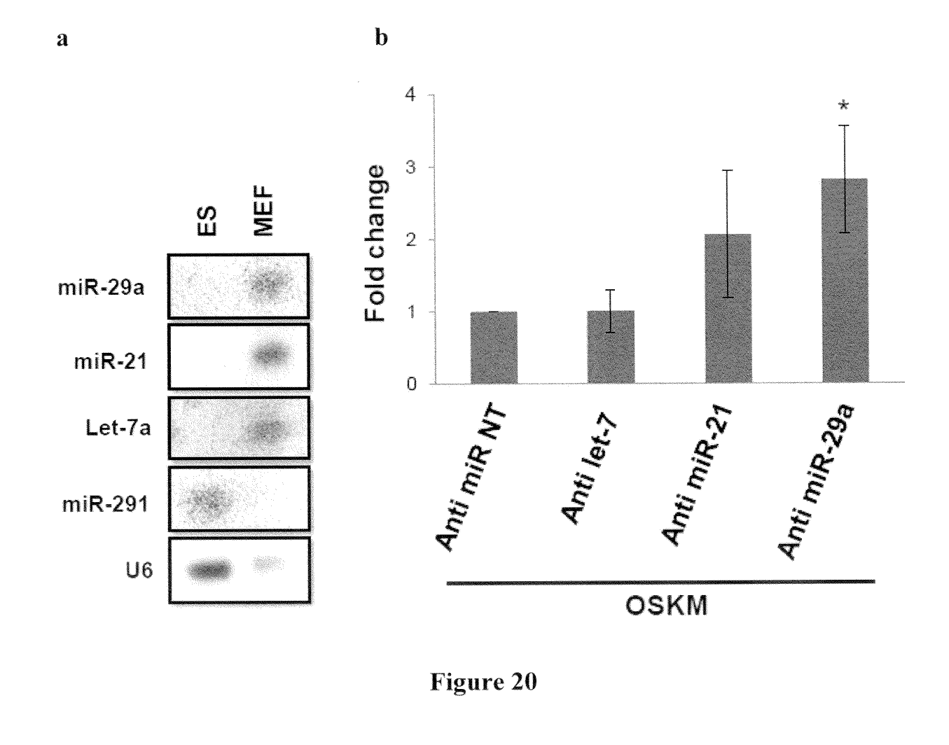

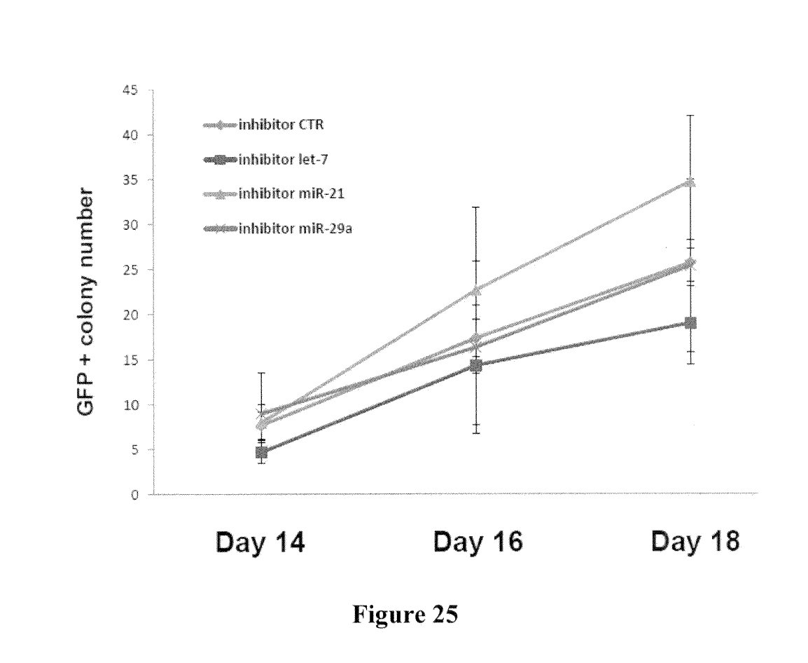

To determine whether inhibiting MEF-specific miRNAs lowers reprogramming barriers, MEF-enriched miRNAs are analyzed and their levels with those seen in mouse ES (mES) cells are compared. As shown in FIG. 20a, let-7a, miR-21, and miR-29a are highly expressed in MEFs compared to mES cells. By contrast, miR 291 is highly abundant in mES but absent in MEFs (FIG. 20a). Next, miRNA inhibitors are introduced against let-7a, miR-21, and miR-29a into Oct4-EGFP MEFs (MEFs harboring Oct4-EGFP reporter) together with retroviruses expressing Oct3/4, Sox2, Klf4, and c-Myc (OSKM). At day 14 post-transduction, cells treated with miR-21 inhibitors show a ˜2.1-fold increase in reprogramming efficiency compared with a non-targeting (NT) control (FIG. 20b). Similarly, reprogramming efficiency increases significantly by ˜2.8-fold following inhibition of miR-29a (FIG. 20b). Under similar antagomir treatments as used for miR 29a or 21 inhibition, a minor effect on OSKM-reprogramming following let-7a inhibition is observed (FIG. 20b). To further test whether miRNA inhibition enhances reprogramming with three factors in the absence of c-Myc, cells are transduced with the miRNA inhibitor together with OSK, which reprograms cells at much lower efficiency than OSKM. The number of OSK-reprogrammed iPS cell colonies increase in the presence of the miR-21 inhibitor relative to treatment with OSK alone (FIG. 25). These results demonstrate that depletion of the MEF-enriched miRNAs miR-21 and miR-29 enhances 4F-reprogramming significantly and that blocking miR-21 moderately increases the efficiency of three factors (OSK) reprogramming.

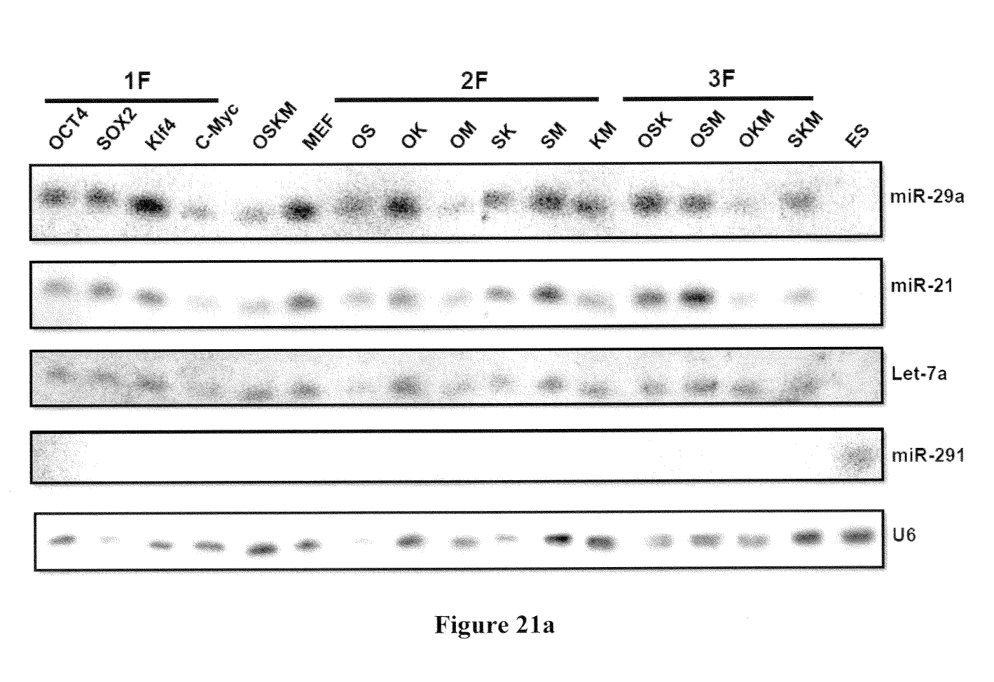

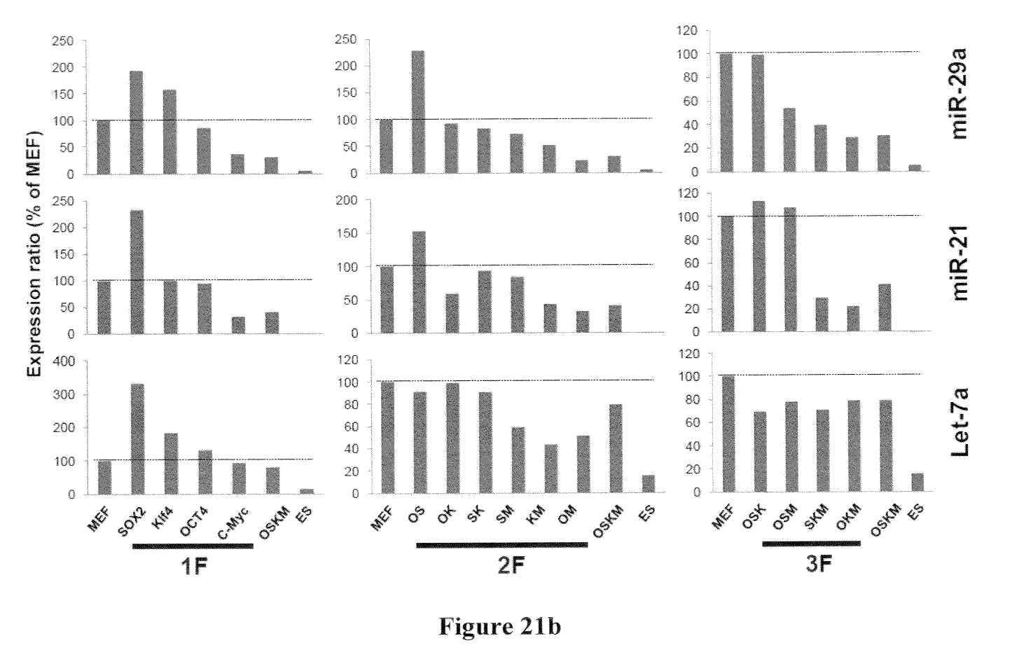

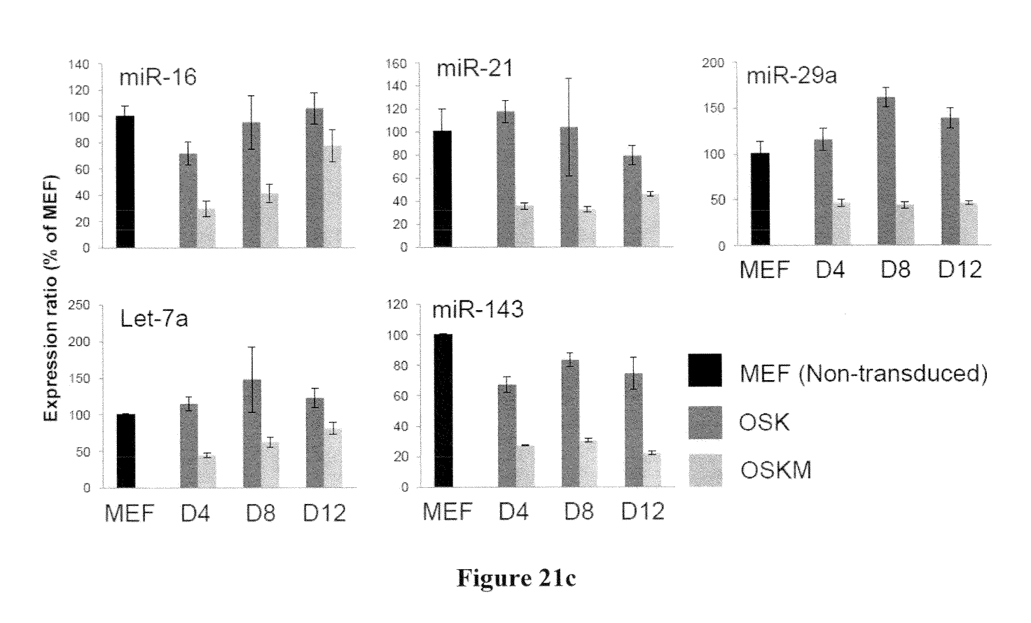

C-Myc represses expression of miRNAs let-7a, miR-16, miR-21, miR-29a, and miR-143 during reprogramming: Recent work indicates that the OSKM factors alter cell identity through both epigenetic and transcriptional mechanisms. The invention provides that OSKM reprogramming factors can down-regulate MEF-enriched miRNAs. To evaluate the potential effect of each reprogramming factor on miRNA expression, MEFs are transduced with various combinations of the OSKM factors and subjected to Northern blot analysis (FIG. 21a). Interestingly, Sox2 alone induce expression level of miR-21, miR-29a, and let-7a by more than two folds, compared with MEF control (FIG. 21b, left panels). Klf4 has minor but similar effect as Sox2 on those select miRNAs (FIG. 21b, left panels). With Oct4 overexpression only, miRNAs do not change expression level (FIG. 21b, left panels). In contrast to Oct4, Sox2, and Klf4, the single factor c-Myc down-regulates expression of miR-21 and miR-29a, the most abundant miRNAs in MEFs, by ˜70% of MEF control (FIGS. 21a and 21b, left panels). Furthermore, among various combinations of two factors (2F) shown in FIG. 21b (middle panels), inclusion of c-Myc can enhance decreases in all three miRNAs, including miR-21, miR-29a, and let-7a, by ˜25-80% (FIG. 21b, middle panels). Similar to 1F effect on miRNAs, Sox2 with Oct4 increase miR-21 and miR-29a by 1.5 fold and 2.3 fold of MEF control, and OK and SK have no obvious effects on miRNA expression. Moreover, among various three-factor (3F) combinations, the expression of miRNA-21 decreases by ˜70 and 78% in SKM and OKM cells, respectively, relative to expression seen in MEFs, and similarly miR-29a expression decreases by ˜48-70% in 3F combinations containing c-Myc (FIG. 21b, right panels). Inclusion of c-Myc in 3F combinations also slightly decreases let-7a levels (FIG. 21b, right panels). OSK without c-Myc had little effect on miRNA expression (FIG. 21b, right panels). Therefore, these results strongly suggest that c-Myc plays an important role in regulating miRNA expression during the reprogramming.

To further confirm that c-Myc is the primary factor antagonizing miRNA expression, cells are transduced with OSK with or without c-Myc, and miRNA expression is examined by real time quantitative reverse transcription polymerase chain reaction (RT-qPCR) at various time points post-transduction. In contrast to OSK, OSKM transduction greatly decreases expression of let-7a, miR-16, miR-21, miR-29a, miR-143 during reprogramming (FIG. 21c), indicating that c-Myc plays a predominant role in regulating expression of MEF-enriched miRNAs, including the most abundant ones, let-7a, miR-21, and miR-29a. These data also suggest that c-Myc boosts reprogramming, in part, through miRNA down regulation.

### Example 13

### IPS Cells Derived via mRNA Depletion Attain Pluripotency

The present invention provides that mouse iPS cells derived with miR-21 and miR-29a inhibitors are pluripotent. Staining with ES cell markers of OSKM/anti miR-29a iPS cells can be performed. GFP+ colonies derived following OSKM and miR-29a inhibitor treatment are picked for further analysis. Representative colonies expressing the embryonic stem cell markers Nanog and SSEA1 are identified. Endogenous Oct4 is also activated, which can be indicated by the EGFP staining. Strong alkaline phosphatase (AP) activity can be observed as one of the ES marker.

In vitro differentiation of OSKM/anti miR-29a iPS cells can be performed. Embryoid bodies can be formed in vitro and cultured for 2 weeks. Cells can be fixed and stained with anti-α fetoprotein (for mesoderm) and anti-beta tubulin III (for ectoderm). Nuclei can be observed as counter stain by Hoescht staining. Teratoma formation analysis of OSKM/anti miR-29a iPS cells can also be performed. 1.5×10⁶ iPSCs are injected subcutaneously into athymic nude female mice. Tumor masses are collected at three weeks after injection and fixed for histopathological analysis. Various tissues derived from three germ layers can be identified, including gut-like epithelium (endoderm), adipose tissue, cartilage, and muscle (mesoderm), and neural tissue and epidermis (ectoderm). Chimera analysis of OSKM/anti miR-29a and OSK/anti miR-21 iPS cells can also be performed. 8 to 14 iPS cells can be injected into E3.5 mouse blastocysts. iPS cell contribution to each chimera can be estimated by assessing black coat color and can be observed as a percentage.

To investigate whether blocking miR-21 or miR-29a compromises iPS cell pluripotency, iPS cells with OSKM/anti miR-29a or OSK/anti miR-21 are evaluated for pluripotency. First, cells are manually picked approximately two weeks after reprogramming and expanded to examine morphology and expression of ES-specific markers. Cells exhibit an ES-like morphology and highly expressed Oct4-EGFP (indicating establishment of endogenous ES cell signaling. In addition, OSKM/anti miR-29a or OSK/anti miR-21 IPS cells express ES cell-specific markers, including Nanog and SSEA1, and exhibited alkaline phosphatase activity. To test whether those iPS cells show pluripotent potential comparable to normally derived iPS cells, OSKM/anti miR-29a and OSK/anti miR-21 iPS cells are induced to form embryoid bodies (EBs) or are injected into nude mice and allowed to differentiate into various tissues. After two weeks of in vitro differentiation, typical cell types derived from all three germ layers are observed. Teratoma tumors, formed three weeks post injection, are subjected to histopathological analysis. Various tissues originating from all three germ layers are generated, confirming that iPS cells obtain pluripotency. To use the most stringent test of pluripotency, iPS cells are injected into E3.5 blastocysts to create chimeric mice. Mice derived from miR-depleted iPS cells show a significant ˜15% to 25% black coat color attributable to iPS cells. These data show that depleting miR-21 and miR-29a has no adverse effect on pluripotency of derived IPS cells.

### Example 14

Inhibiting MiR-29a Down-Regulates P53 Through P85α and CDC42 Pathways

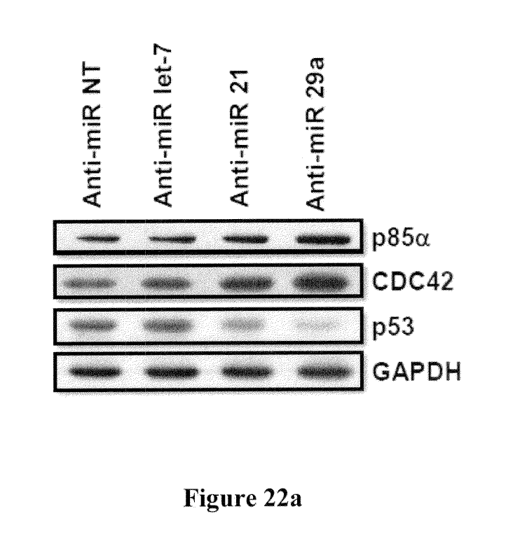

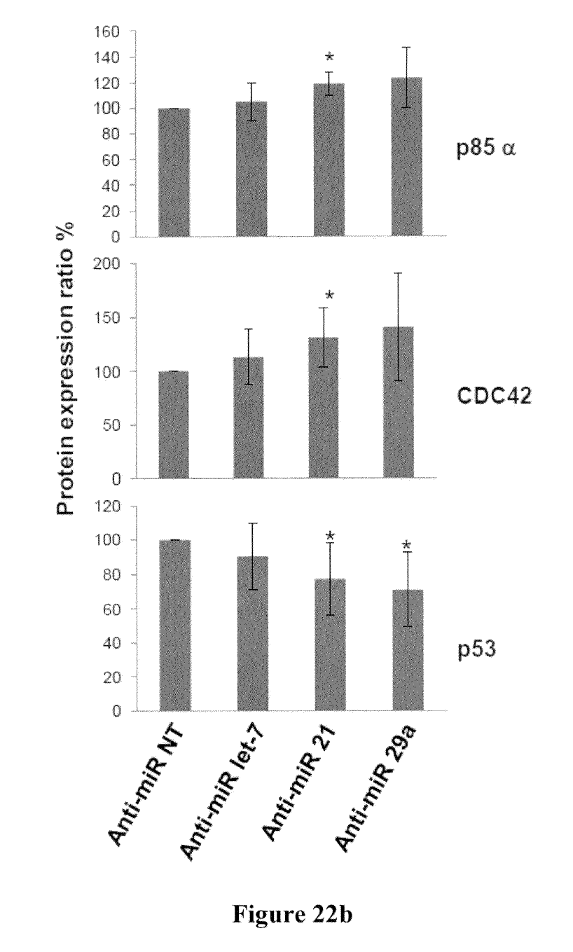

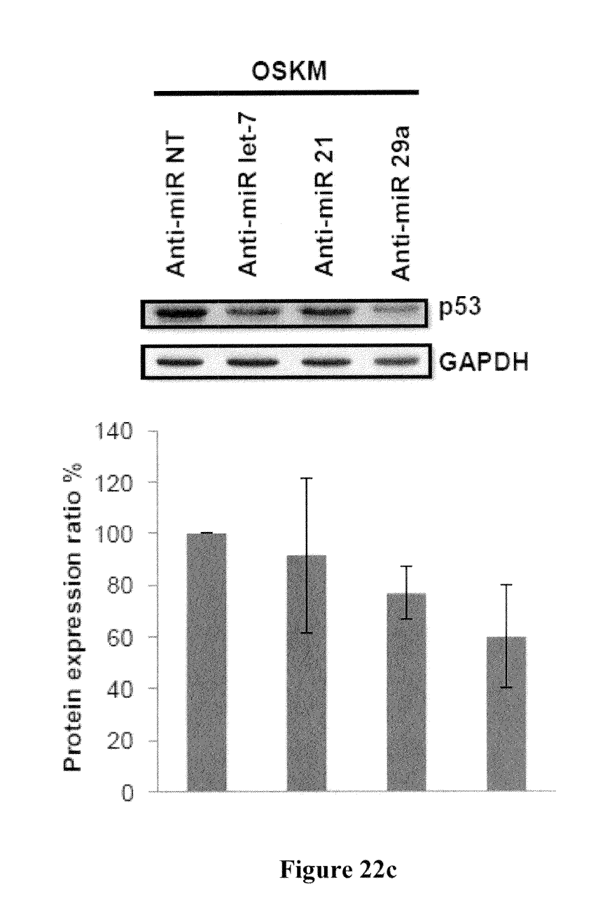

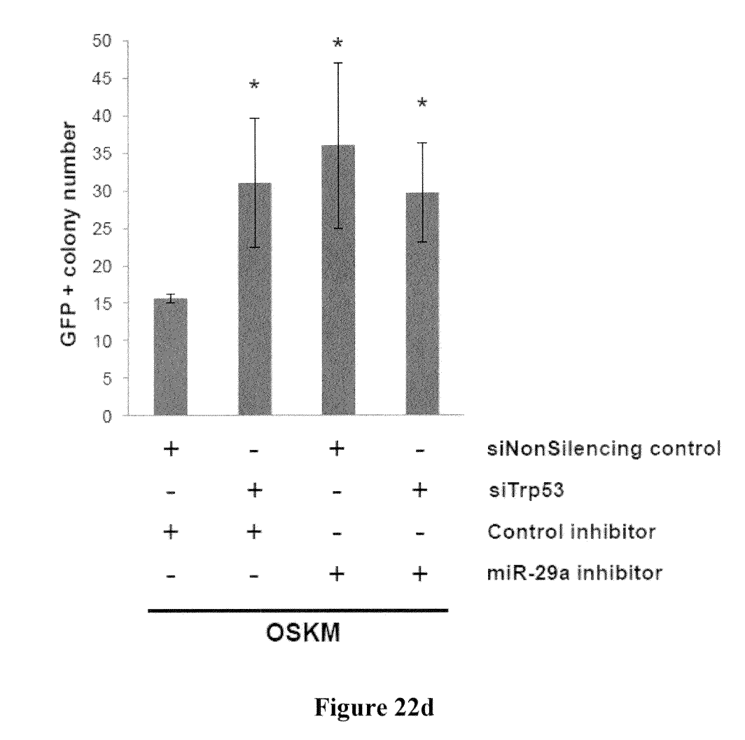

To understand mechanisms underlying miR-29a′ s effect on reprogramming, expressions of p85α and CDC42 are examined, where p85α and CDC42 are reportedly direct targets of miR-29 in HeLa cells. To do so, miRNA inhibitors are transfected into MEFs and p85α and CDC42 protein expression are evaluated by western blot at day 5 post-transfection. P85α and CDC42 protein levels increase slightly following miR-29a block, whereas a let-7a inhibitor has little effect (FIGS. 22a and 22b). The transformation related protein 53 (Trp53 or p53) is also reportedly a direct target of p85α and CDC4. Therefore, p53 is examined whether it's indirectly regulated by miR-29a in MEFs as well. To test that, MEFs are transfected with miRNA inhibitors and harvested five days for immunoblotting to evaluate expression of p53. P53 protein levels decreases by ˜30% (FIGS. 22a and 22b) following miR-29a inhibition but are not altered by the NT control or by let-7a inhibition. Significantly, depleting miR-21 also releases p85α and CDC42 protein repression and consequently the levels of p85α and CDC42 increase, which results in down regulation of p53 expression by ˜25% (FIGS. 22a and 22b).

To further confirm that p53 levels decrease with inhibition of miR-21 or miR-29a during reprogramming, p53 expression is examined at reprogramming day 5 by western blot analysis. To initiate reprogramming, miRNA inhibitors are introduced together with OSKM. Consistent with observations in MEFs alone, p53 protein levels decrease by ˜25% or ˜40% following miR-21 or miR-29a depletion, respectively, during reprogramming, compared with OSKM controls (FIG. 22c). In summary, our data showed that blocking miR-29a reduced p53 protein levels by ˜30-40% through p85α and CDC42 pathways during reprogramming. In addition, depletion of miR-21 has a similar effect on both p85α and CDC42 and lowered p53 protein levels by ˜25% to ˜30%.

Inhibition of miR-29a enhances reprogramming efficiency through p53 down-regulation: It is reported that p53 deficiency can greatly increase reprogramming efficiency. Since depleting miR-29a significantly decreases p53 levels and increases reprogramming efficiency by ˜2.8-fold, the invention provides that the effect of miR-29a knockdown is mediated primarily by p53 down-regulation. To that end, p53 siRNA and/or the miR-29a inhibitor is transfected into Oct4-EGFP MEFs together with OSKM to initiate reprogramming. Down-regulation of p53 by siRNA (−80%) has a similar positive effect on reprogramming efficiency as does miR-29a inhibition (FIG. 22d). No increase in reprogramming efficiency is observed when miR inhibitors are added in the presence of p53 siRNA (FIG. 22d). These results suggest that inhibition of miR-29a acts, in part, through down-regulation of p53 to increase reprogramming efficiency.

### Example 15

### Inhibition of MiR-21 and MiR-29a Decreases Phosphorylation of ERK1/2, but not GSK3β, to Enhance Reprogramming

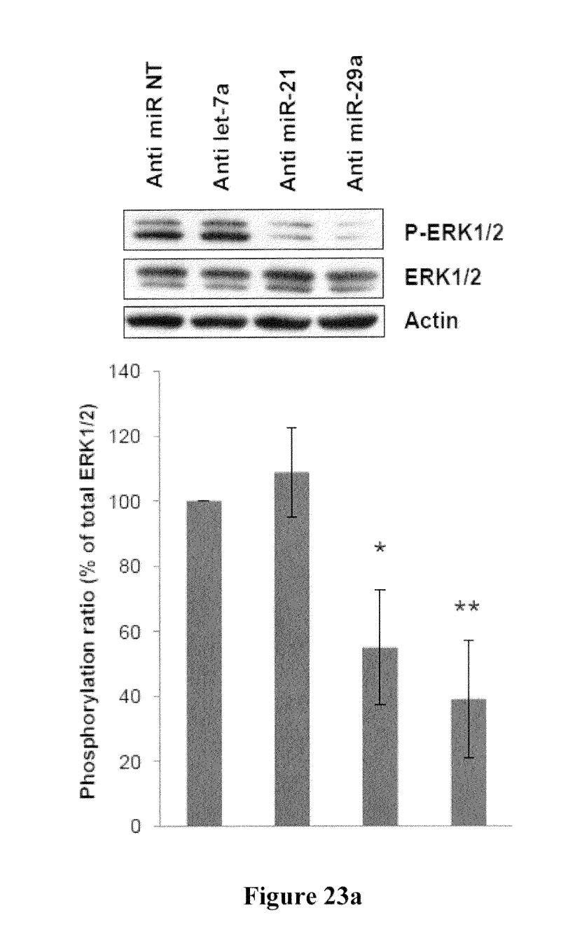

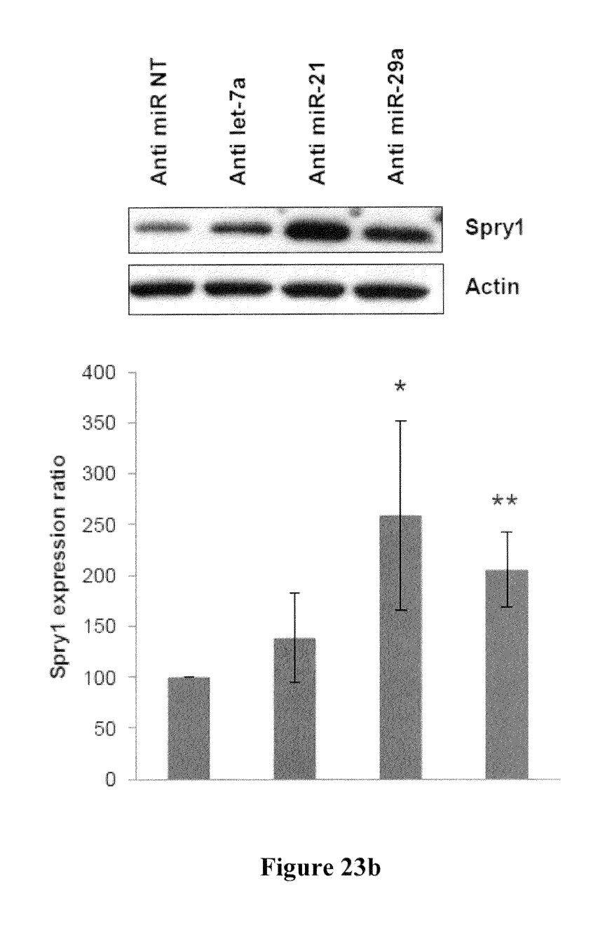

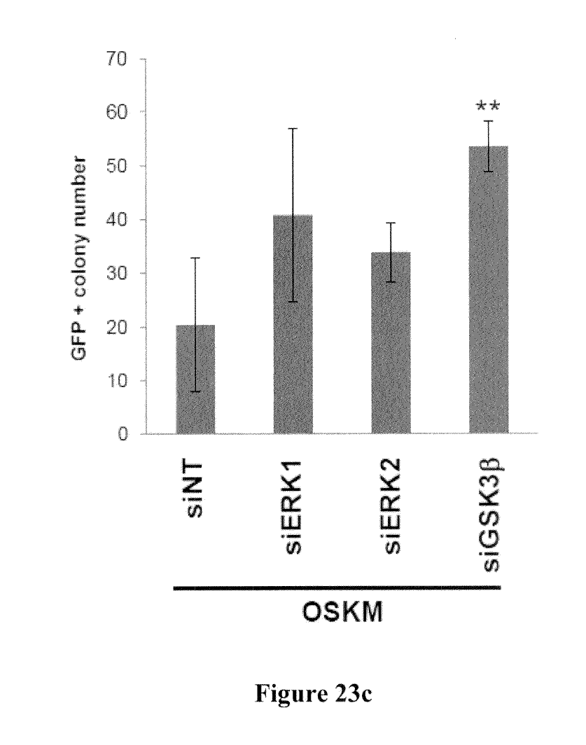

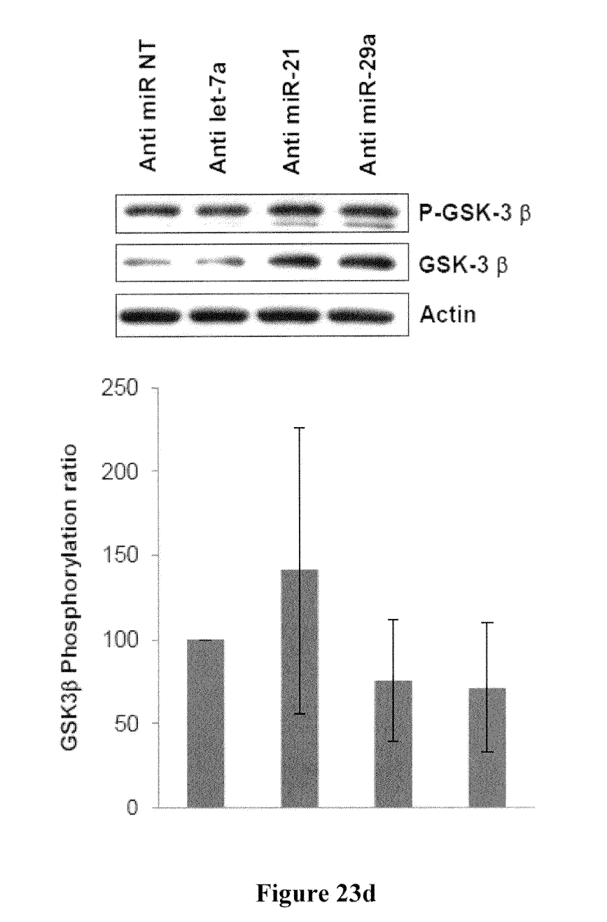

MiR21 reportedly activates MAPK/ERK through inhibition of the sprouty homologue 1 (Spry1) in cardiac fibroblasts. Blocking MAPK/ERK activity promotes reprogramming of neural stem cells and secures the ground state of ESC self-renewal. Therefore, the invention provides that miR-21 regulates the MAPK/ERK pathway during reprogramming by evaluating ERK1/2 phosphorylation in MEFs following introduction of miRNA inhibitors. To test that, MEFs are transfected with miRNA inhibitors and then harvested for Western blot analysis to determine the phosphorylated ERK1/2 level. Western blot analysis shows that blocking miR-21 significantly decreased by ˜45% ERK1/2 phosphorylation relative to NT controls, while let-7a inhibitors have no such effect (FIG. 23a). Interestingly, depleting MEFs of miR-29a also significantly reduces ERK1/2 phosphorylation by 60% relative to NT control (FIG. 23a). The invention also provides that miR-21 and miR-29a can affect ERK1/2 phosphorylation by altering Spry1 levels. MiR-21 or miR-29a are depleted in MEF by transfecting various miRNA inhibitors and Spry1 expression levels are quantified by immunoblotting and the results show that inhibiting miR-21 and miR-29a enhanced Spry1 expression levels (FIG. 23b). Therefore, depleting miR-21 and miR-29a down-regulates phosphorylation of ERK1/2 by modulating Spry1 protein levels.

To address whether ERK1/2 downregulation enhances reprogramming efficiency, siRNAs targeting ERK1 or 2 are introduced into Oct4-EGFP MEFs in the course of 4F-reprogramming. Depletion of either enhances generation of mature iPS cells (FIG. 23c). The invention provides that miR-21 acts as an inducer of ERK1/2 activation in MEFs, since blocking miR-21 reduces ERK1/2 phosphorylation. Depleting miR-29a also significantly diminishes ERK1/2 phosphorylation. These results strongly suggest that miR-21 and miR-29a regulate ERK1/2 activity to enhance reprogramming efficiency (FIGS. 23a, 23b, and 23c).

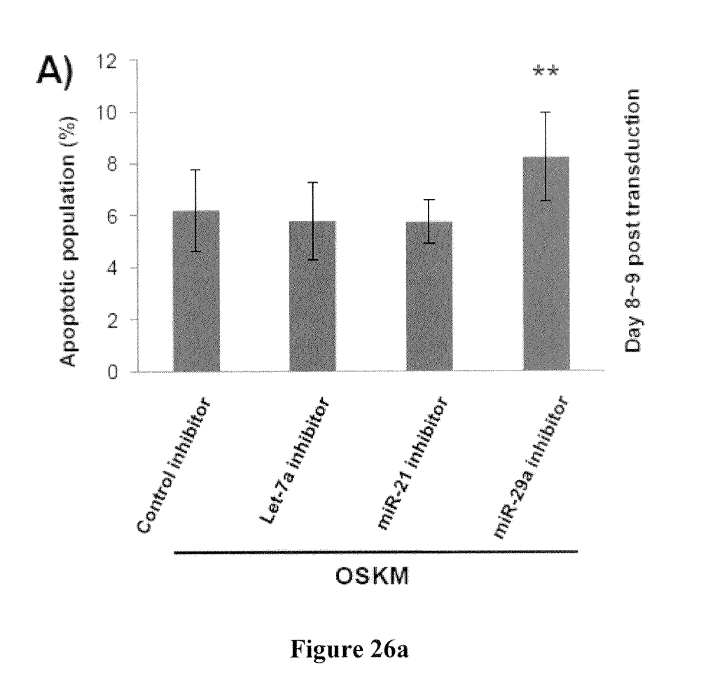

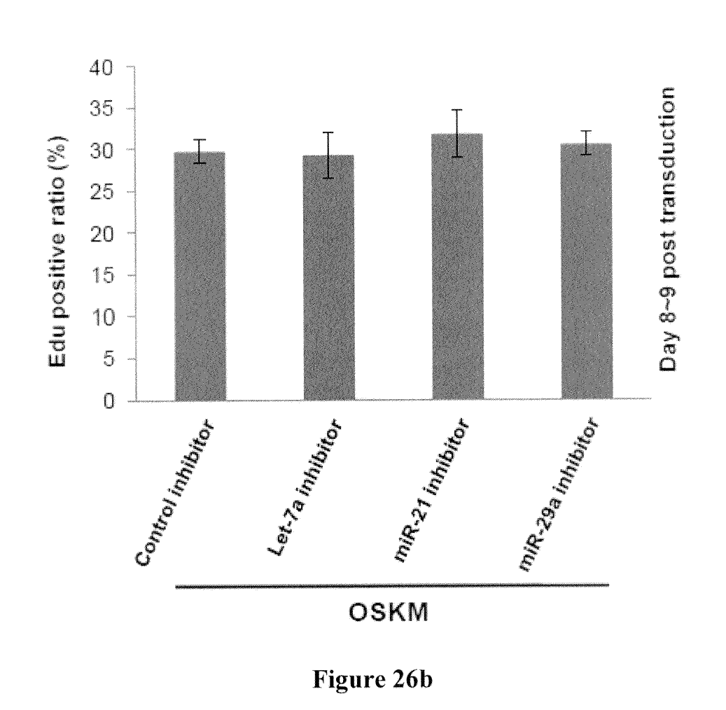

The GSK3β pathway also represses ES self-renewal and reprogramming of neural stem cells. Depleting GSK3β greatly increases mature iPS cell generation (FIG. 23c). The invention provides that miRNA depletion regulated GSK3β activation. Immunoblotting shows that blocking miRNAs in Oct4-EGFP MEFs has no significant effect on GSK3P activation (FIG. 23d). The invention provides that miRNA depletion alters apoptosis or cell proliferation during reprogramming by using flow cytometry to assess cell viability and replication rate. Blocking miRNAs 21, 29a, or let-7 during reprogramming with OSKM does not alter apoptosis or proliferation rates (FIG. 26). Overall, miR-29a and miR-21 modulate p53 and ERK1/2 pathways to regulate iPS cell reprogramming efficiency.

### Example 16

C-Myc Reduces the Threshold for Reprogramming by Decreasing P53 Levels and Antagonizing ERK1/2 Activation Through MiR-21 and MiR-29a Down-Regulation

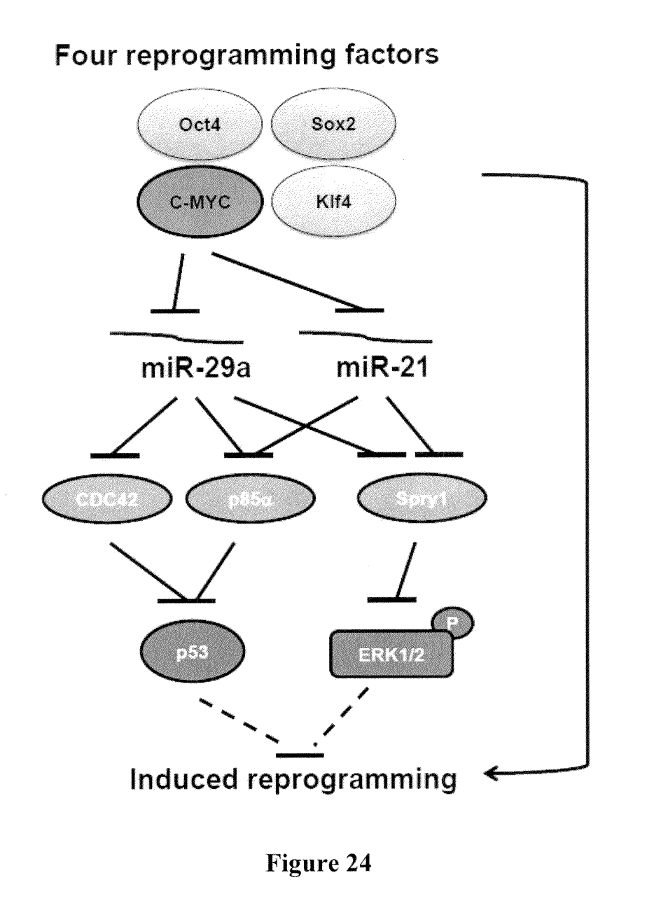

To develop alternatives for transgenes currently used for induced-reprogramming, it is crucial to understand how signaling pathways are regulated by these factors. The invention provides that c-Myc represses MEF-enriched miRNAs, such as miR-21, let-7a, and miR-29a, during reprogramming (FIG. 20). Depleting miR-29a with inhibitors decrease p53 protein levels most likely by releasing p85α and CDC42 repression (FIG. 22). In addition, depleting miR-21 decreases ERK1/2 phosphorylation (FIG. 23). The invention provides that miR-21 inhibition reduces p53 protein levels and that inhibiting miR-29a also reduces ERK1/2 phosphorylation level. Both p53 and ERK1/2 signaling antagonizes reprogramming. Blocking miR-21 and miR-29a or knockdown of p53 and ERK1/2 can enhance reprogramming efficiency (FIGS. 22 and 23). The invention provides that c-Myc facilitates reprogramming in part by suppressing the MEF-enriched miRNAs, miR-21 and miR-29a, which act as reprogramming barriers through induction of p53 protein levels and ERK1/2 activation (FIG. 24).

Forced expression of ES-specific miRNAs of the miR-290 family can replace c-Myc to promote reprogramming. C-Myc also binds the promoter region of the miR-290 cluster. However, early expression of the c-Myc transgene is effective to initiate reprogramming but dispensable at the transition stage or later in mature iPS cells where miR-290 clusters start to express. Therefore, it is unlikely that c-Myc promotes early stages of reprogramming through activating the miR-290 family.

The invention provides that expression level of MEF-enriched miRNAs, including miR-29a, miR-21, miR-143 and let-7a, decreases when c-Myc is introduced for reprogramming. C-Myc has a profound transcriptional effect on miRNAs in promoting tumorigenesis or sustaining the pluripotency ground state. Therefore, c-Myc repression of miRNA expression is the likely mechanism underlying reprogramming.

MiR-21 acts as positive mediator to enhance fibrogenic activity through the TGFβ1 and ERK1/2 pathways, both of which have been shown to influence reprogramming and the ES cell ground state. Notably, among validated miR-29a targets, p53 is positively regulated by miR-29a. In addition, recent studies show that the Ink4-Arf/p53/p21 pathway compromises reprogramming, and p53 deficiency greatly enhances reprogramming efficiency. Thus, these signaling pathways are likely the primary barriers to the reprogramming process.

Depleting the c-Myc-targeted miRNAs, miR-21 and miR-29a, enhances reprogramming efficiency ˜2.1- to ˜2.8-fold (FIG. 20), suggesting that MEF-enriched miRNAs also function as reprogramming barriers. Let-7 inhibition has been recently reported to enhance reprogramming, however, by several attempts only a minor effect in reprogramming is observed when let-7 is inhibited by antagomirs (FIG. 20). Moreover, the invention provides that the induction of p53 during reprogramming is compromised by miR-29a inhibition, enhancing reprogramming efficiency. Similarly, reprogramming can be greatly promoted by either depleting miR-21 or ERK1/2. C-Myc is a major contributor to the early stage of reprogramming and is not required to sustain the process at transition and late stages, indicating that c-Myc-regulated miRNAs may be employed to initiate high efficiency reprogramming.

C-Myc reportedly directly binds to and represses the miR-29a promoter. The invention provides that c-Myc can be only partially replaced by depleting miR-21 and suggest that c-Myc has other functions in reprogramming. Thus, regulation of multiple pathways or wide repression of MEF-enriched miRNAs may be required to replace c-Myc function during reprogramming.

The invention provides that c-Myc reduces the threshold for reprogramming by decreasing p53 levels and antagonizing ERK1/2 activation through miR-21 and miR-29a downregulation. Additionally, factors downstream of c-Myc may serve as targets for manipulation by siRNA, miRNA, or small molecules, to improve reprogramming. These approaches can be extended to replace all four reprogramming factors.

Although the invention has been described with reference to the above example, it will be understood that modifications and variations are encompassed within the spirit and scope of the invention. Accordingly, the invention is limited only by the following claims.

1. A method of generating an induced pluripotent stem (iPS) cell comprising:
a) contacting a somatic cell with a nuclear reprogramming factor; and
b) contacting the cell of (a) with a microRNA that alters RNA levels or activity within the cell, thereby generating an iPS cell.

2. The method of claim 1, wherein the microRNA or RNA is modified.

3. The method of claim 1, wherein the microRNA is in a vector.

4. The method of claim 1, wherein the microRNA is in the miR-17, miR-25, miR-106a, miR let-7 family member or miR-302b cluster.

5. The method of claim 1, wherein the microRNA is miR-93, miR-106b, miR-21, miR-29a, or a combination thereof.

6. The method of claim 1, wherein the microRNA has a polynucleotide sequence comprising SEQ ID NO: 1.

7. The method of claim 1, wherein the microRNA has a polynucleotide sequence selected from the group consisting of SEQ ID NOs: 2-11.

8. The method of claim 1, wherein the microRNA regulates expression or activity of p21, Tgfbr2, p53, Ago2, or a combination thereof.

9. The method of claim 1, wherein the microRNA regulates Spry 1/2, p85, CDC42, or ERK1/2 pathways.

10. The method of claim 1, wherein the nuclear reprogramming factor is encoded by a gene contained in a vector.

11. The method of claim 1, wherein the nuclear reprogramming factor is a SOX family gene, a KLF family gene, a MYC family gene, SALL4, OCT4, NANOG, LIN28, or a combination thereof.

12. The method of claim 1, wherein the nuclear reprogramming factor is one or more of OCT4, SOX2, KLF4, C-MYC.

13. The method of claim 1, wherein the nuclear reprogramming factor comprises c-Myc.

14. The method of claim 1, wherein the somatic cell is contacted with the reprogramming factor prior to, simultaneously with or following contacting with the microRNA.

15. The method of claim 1, wherein the somatic cell is a mammalian cell.

16. An induced pluripotent stem (iPS) cell produced using the method of claim 1.

17. An enriched population of induced pluripotent stern (iPS) cells produced by the method of claim 1.

18. A differentiated cell derived by inducing differentiation of the pluripotent stem cell produced by the method of claim 1.

19. A method of treating a subject comprising:
a) generating an induced pluripotent stem (iPS) cell from a somatic cell of the subject by the method of claim 1;
b) inducing differentiation of the iPS cell of step (a); and
c) introducing the cell of (b) into the subject, thereby treating the condition.

20. The use of microRNA for increasing efficiency of generating of iPS cells.

21. The use of claim 20, wherein the microRNA is selected from the group consisting of miR-17, miR-25, miR-93, miR-106a, miR-106b, miR-21, miR-29a, miR-302b cluster, miR let-7 family member or a combination thereof.

22. A combination of miR sequences selected from the group consisting of an least two or more of miR-17, miR-25, miR-93, miR-106a, miR-106b, miR-21, miR-29a, miR-302b cluster, miR let-7 family member, or a combination thereof.

23. A method of generating an induced pluripotent stem (iPS) cell comprising:
a) contacting a somatic cell with a nuclear reprogramming factor; and
b) contacting the cell of (a) with an inhibitor of microRNA, thereby generating an iPS cell.

24. The method of claim 23, wherein the microRNA is in the miR-17, miR-25, miR-106a, miR let-7 family member or miR-302b cluster.

25. The method of claim 23, wherein the microRNA is miR-93, miR-106b, miR-21, miR-29a, or a combination thereof.

26. The method of claim 23, wherein the microRNA regulates expression or activity of p21, Tgfbr2, p53, Ago2, or a combination thereof.

27. The method of claim 23, wherein the nuclear reprogramming factor is a SOX family gene, a KLF family gene, a MYC family gene, SALL4, OCT4, NANOG, LIN28, or a combination thereof.

28. The method of claim 23, wherein the somatic cell comprises a fibroblast.

29. An induced pluripotent stem (iPS) cell produced using the method of claim 23.
